(19) United States
(12) Patent Application Publication
(10) Pub. No.: US 2022/0017462 A1
(43) Pub. Date: Jan. 20, 2022

(54) ORTHO-PHTHALALDEHYDE CONTAINING LINKERS AND USE FOR PREPARATION OF ANTIBODY-DRUG CONJUGATE

(71) Applicants: Wuxi Biologics Ireland Limited, Dublin 1 (IE); Versitech Limited, Hong Kong (CN)

(72) Inventors: Xuechen LI, Hong Kong (CN); Mingzhi JIN, Shanghai (CN); Yue ZHANG, Shanghai (CN); Li YIN, Shanghai (CN); Jun WANG, Shanghai (CN)

(21) Appl. No.: 17/296,088

(22) PCT Filed: Nov. 21, 2019
(86) PCT No.: PCT/CN2019/119884

§ 371 Date: May 21, 2021

(30) Foreign Application Priority Data
Nov. 23, 2018 (CN) PCT/CN2018/117257

(51) Int. Cl. C07D 207/452; C07C 235/78; C07D 241/04; C07C 233/61; C07K 1/113; A61K 47/68

(52) U.S. Cl. CPC C07D 207/452; C07C 235/78; A61K 47/6889; C07C 233/61; C07K 1/113; C07D 241/04

(57) ABSTRACT

Provided herein are novel ortho-Phthalaldehyde (OPA) containing linkers (OPA-L) and the use of OPA-L for the preparation of Antibody-drug conjugate (ADC) via the formation of Phthalimidine through the reaction of primary amine on antibody (e.g., residue of Lysine) and ortho-Phthalaldehyde. The advantage of this OPA-L is high reactivity and can be applied in different types of antibodies to form stably-linked conjugates. The use of OPA-L for the preparation of ADC is advantageous for mild and wide condition of conjugation, for instance, low percentage of organic solvent required, wide range of pH and temperature can be used.

### FIELD OF INVENTION

The present invention relates to a novel ortho-Phthalaldehyde (OPA) containing linkers (OPA-L) and the use of OPA-L for the preparation of Antibody-drug conjugate (ADC) via the formation of Phthalimidine through the reaction of primary amine on antibody (e.g., residue of Lysine) and ortho-Phthalaldehyde.

### BACKGROUND OF THE INVENTION

Antibody-drug conjugate (ADC) is a novel targeted drug for disease treatment. ADC contains an antibody for targeting, a connector and linker for drug attachment and a high potent payload as effector. Since the approvals of Adcetris in 2011 and Kadcyla in 2013 by US FDA, ADC drug development has widely spread for the treatment of cancer.

For drug attachment, functional groups with high reactivity and stability on both antibody and linker-payload (i.e., liner-drug) were used for the coupling, to form stable covalent bonds. Conventional antibody-drug conjugates are usually produced by two chemical strategies, Lysine based conjugation and Cysteine from the reduction of interchain sulfide bonds based conjugation. For the reaction of primary amine group on Lysine residue, the most widely used connector on linker-payload is the NHS ester (i.e., N-hydroxysuccinimide). But the application of NHS ester in antibody-drug conjugate production is limited by its inherent properties, for instance, the reaction between NHS ester and primary amine is very slow under acidic conditions, so the conjugation needs to be performed in the buffer with high pH value (i.e., >7.0), which is not friendly to antibody sometimes, and the NHS is prone to hydrolysis under basic conditions, which makes the purification and identification of free drug after conjugation more complicated. Also, due to the low reactivity of NHS ester to primary amine on antibody, the reaction needs to be carried out with high temperature (i.e., 22° C.). Even more, due to the low solubility, more organic solvent is required for linker-payload prepared by NHS ester (i.e., SMCC-DM1) to be fully dissolved in the reaction systems, which increases the risk of aggregation of antibody.

Based on the unique biophysical, biochemical and pharmacological characteristics, Heavy Chain Antibodies (HcAb) become more and more important in antibody drug discovery. But due to the small size and lack of Lysine residue in HcAb, it is hard to get high drug-antibody ratio (DAR) for ADC using the NHS ester containing linker-payload.

Toxicity of ADC drugs caused by the instability in circumstance is another important issue for the application in disease treatment. The degradation of linker in circumstance causes the release of toxic drug from antibody and leads to off-target toxicity. The current linkers (i.e., SMCC) have this instability issue in circumstance. ADCs produced by this kind of linkers are not stable in plasma, thus they may have poor properties in pharmacology and toxicology.

Therefore, there is an urgent need in the prior art for providing a linker with high reactivity, high stability, high solubility and friendly to antibodies for conjugation, which is beneficial for the forming of ADC.

### SUMMARY OF THE DESCRIPTION

The inventor of the present application surprisingly found that the OPA-Ls described herein are more stable than the amide bond formed by the NHS ester, broad conjugation conditions can be tested by using OPA-Ls, and ADCs produced with OPA-Ls have better plasma stability compared to ADCs produced with NHS ester containing linker. In short, the OPA-Ls described herein possess high reactivity, high stability, high solubility and are friendly to antibodies for conjugation and to HcAb based conjugation as well.

In the first aspect of the present application relates to a compound of the following formula (I): OPA-L (I), wherein OPA is

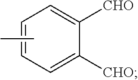

L includes alkyl, alkenyl, alkynyl, cycloalkyl, cycloalkenyl, cycloalkynyl, heterocycloalkyl, heterocycloalkenyl, heterocycloalkynyl, aryl, heteroaryl, alkylene, alkenylene, alkynylene, arylene, heteroarylene, cycloalkylene, heterocycloalkylene; —(CH2)m-, wherein one or more CH2 are replaced by one or more groups selected from NH and C═O, m is an integer of 0-20; —(CH2)n-heteroaryl, wherein one or more CH2 are replaced by one or more groups selected from —NH, —C═O and —O—, n is an integer of 0-20; —(CH2)o-heterocycloalkyl-(CH2)o-CH3, wherein one or more CH2 are replaced by one or more groups selected from NH and C═O, o is an integer of 0-20; —(CH2)q-cycloalkyl-(CH2)q-CH3, wherein one or more CH2 are replaced by one or more groups selected from NH and C═O, q is an integer of 0-20; —(CH2)r-cycloalkyl-(CH2)r-heteroaryl, wherein one or more CH2 are replaced by one or more groups selected from NH and C═O, r is an integer of 0-20; peptide like di-peptides, tri-peptides, tetra-peptide, penta-peptide; oligosaccharide, polyethylene glycol (PEG), and the combinations thereof, and said alkyl, alkenyl, alkynyl, cycloalkyl, cycloalkenyl, cycloalkynyl, heterocycloalkyl, heterocycloalkynyl, aryl, heteroaryl, —CH3, heterocycloalkenyl, alkylene, alkenylene, alkynylene, arylene, heteroarylene, cycloalkylene, heterocycloalkylene are optionally substituted by at least one substituents.

In some embodiments, L includes —(CH2)m-, wherein one or more CH2 are replaced by one or more groups selected from NH and C═O, m is an integer of 0-15; —(CH2)n-heteroaryl, wherein one or more CH2 are replaced by one or more groups selected from NH, C═O and —O—, n is an integer of 0-20, and the heteroaryl group has 5 to 10 ring members; —(CH2)o-C₃₋₇heterocycloalkyl-(CH2)o-CH3, wherein one or more CH2 are replaced by one or more groups selected from NH and C═O, o is an integer of 0-15; —(CH2)q-C₃₋₇cycloalkyl-(CH2)q-CH3, wherein one or more CH2 are replaced by one or more groups selected from NH and C═O, q is an integer of 0-15; —(CH2)r-C₃₋₇cycloalkyl-(CH2)r-heteroaryl, wherein one or more CH2 are replaced by one or more groups selected from NH and C═O, r is an integer of 0-15, and the heteroaryl group has 5 to 10 ring members, and said cycloalkyl, heterocycloalkyl, —CH3 and heteroaryl are optionally substituted by oxo and halogen.

In some embodiments, wherein L includes —(CH2)m-, wherein one or more CH2 are replaced by one or more groups selected from NH and C═O, m is an integer of 0-10; —(CH2)n-heteroaryl, wherein one or more CH2 are replaced by one or more groups selected from NH, C═O and —O—, n is an integer of 0-20, and the heteroaryl has

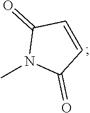

—(CH2)o-piperazinyl-(CH2)o-CH3, wherein one or more CH2 are replaced by one or more groups selected from NH and C═O, o is an integer of 0-10; —(CH2)q-cyclohexyl-(CH2)q-CH3, wherein one or more CH2 are replaced by one or more groups selected from NH and C═O, q is an integer of 0-10; —(CH2)r-cyclohexyl-(CH2)r-heteroaryl, wherein one or more CH2 are replaced by one or more groups selected from NH and C═O, r is an integer of 0-10, and the heteroaryl has

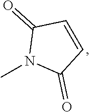

and said —CH3- is optionally substituted by halogen.

In some embodiments, wherein L includes —(CH2)m-, wherein no more than CH2 are replaced by one or more groups selected from NH and C═O, m is an integer of 0-10; —(CH2)n-heteroaryl, wherein no more than eight CH2 are replaced by one or more groups selected from NH, C═O and —O—, n is an integer of 0-18, and the heteroaryl has

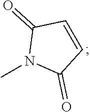

—(CH2)o-piperazinyl-(CH2)o-CH3, wherein one CH2 is replaced by one group selected from NH and C═O, o is an integer of 0-5; —(CH2)q-cyclohexyl-(CH2)q-CH3, wherein no more than six CH2 are replaced by one or more groups selected from NH and C═O, q is an integer of 0-8; —(CH2)r-cyclohexyl-(CH2)r-heteroaryl, wherein no more than five CH2 are replaced by one or more groups selected from NH and C═O, r is an integer of 0-6, and the heteroaryl has

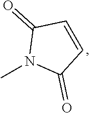

and said —CH3- is optionally substituted by Cl.

In some embodiments, wherein the compound is selected from the group consisting of:

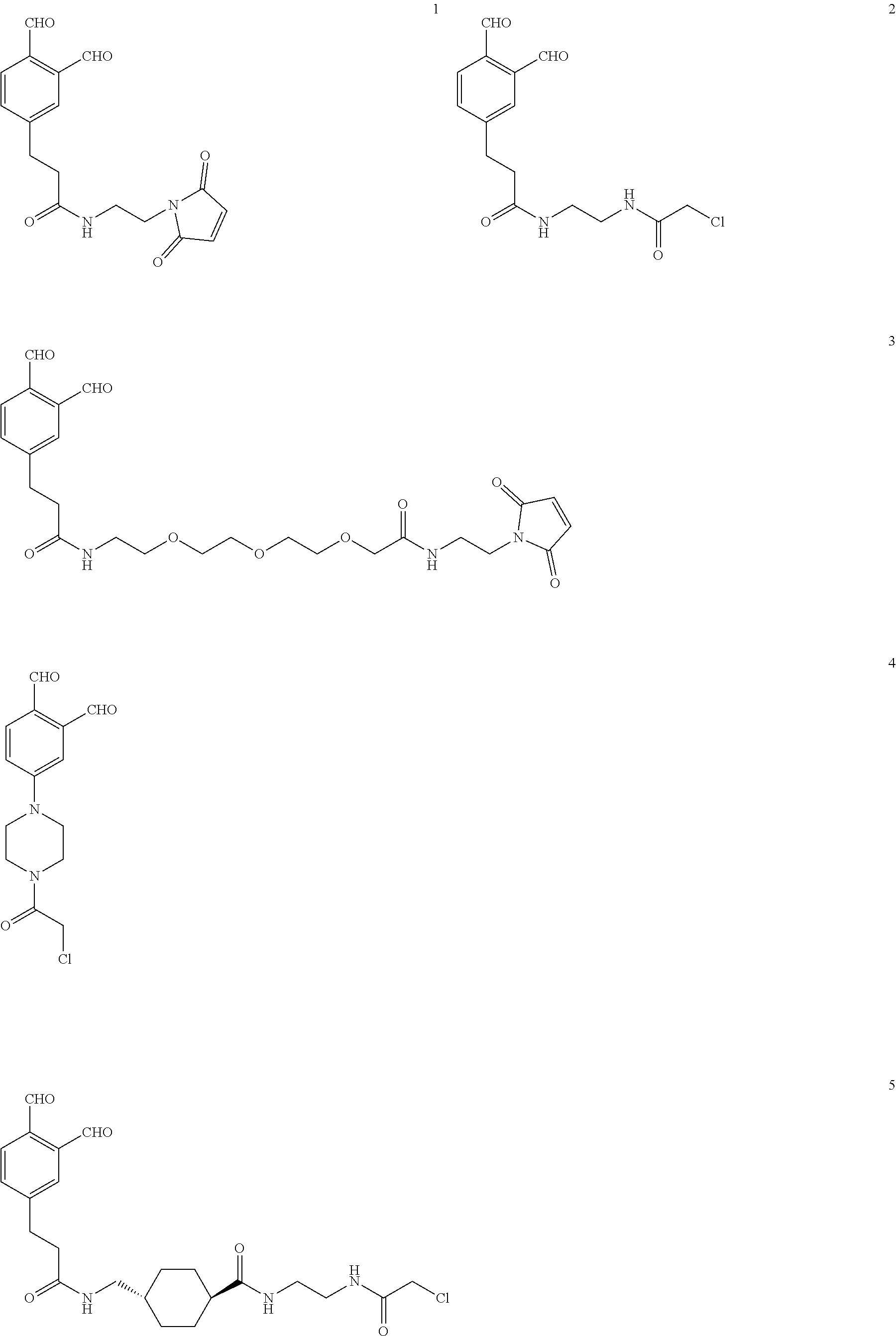 [SMILES: O=Cc1ccc(CCC(=O)NCCN2C(=O)C=CC2=O)cc1C=O ; O=Cc1ccc(CCC(=O)NCCNC(=O)CCl)cc1C=O ; O=Cc1ccc(CCC(=O)NCCOCCOCCOCC(=O)NCCN2C(=O)C=CC2=O)cc1C=O ; O=Cc1ccc(N2CCN(C(=O)CCl)CC2)cc1C=O ; O=Cc1ccc(CCC(=O)NC[C@H]2CC[C@H](C(=O)NCCNC(=O)CCl)CC2)cc1C=O]

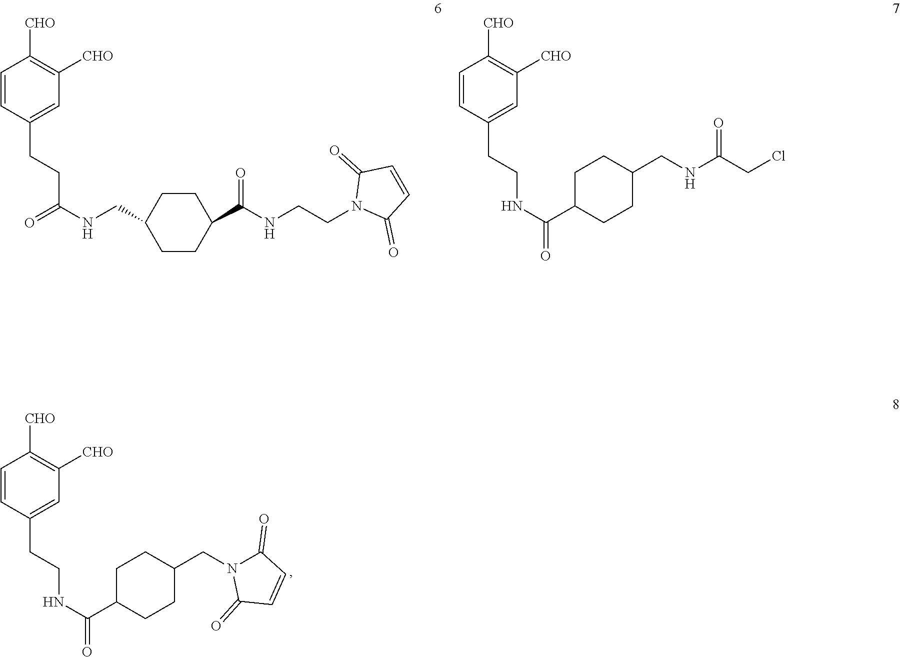 [SMILES: O=Cc1ccc(CCC(=O)NC[C@H]2CC[C@H](C(=O)NCCN3C(=O)C=CC3=O)CC2)cc1C=O ; O=Cc1ccc(CCNC(=O)C2CCC(CNC(=O)CCl)CC2)cc1C=O ; O=Cc1ccc(CCNC(=O)C2CCC(CN3C(=O)C=CC3=O)CC2)cc1C=O]

In the second aspect of the present application relates to a compound of the following formula (II): OPA-L-D (II), wherein OPA is

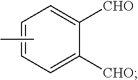

L includes alkyl, alkenyl, alkynyl, cycloalkyl, cycloalkenyl, cycloalkynyl, heterocycloalkyl, heterocycloalkenyl, heterocycloalkynyl, aryl, heteroaryl, alkylene, alkenylene, alkynylene, arylene, heteroarylene, cycloalkylene, heterocycloalkylene; —(CH2)m-, wherein one or more CH2 are replaced by one or more groups selected from NH and C═O, m is an integer of 0-20; —(CH2)n-heteroaryl, wherein one or more CH2 are replaced by one or more groups selected from NH, C═O and —O—, n is an integer of 0-20; —(CH2)o-heterocycloalkyl-(CH2)o-CH3, wherein one or more CH2 are replaced by one or more groups selected from NH and C═O, o is an integer of 0-20; —(CH2)q-cycloalkyl-(CH2)q-CH3, wherein one or more CH2 are replaced by one or more groups selected from NH and C═O, q is an integer of 0-20; —(CH2)r-cycloalkyl-(CH2)r-heteroaryl, wherein one or more CH2 are replaced by one or more groups selected from NH and C═O, r is an integer of 0-20; peptide; oligosaccharide, polyethylene glycol (PEG), and the combinations thereof, and said alkyl, alkenyl, alkynyl, cycloalkyl, cycloalkenyl, cycloalkynyl, heterocycloalkyl, heterocycloalkynyl, aryl, heteroaryl, —CH3, heterocycloalkenyl, alkylene, alkenylene, alkynylene, arylene, heteroarylene, cycloalkylene, heterocycloalkylene are optionally substituted by at least one substituents; D is independently active reagent, wherein said active reagent includes an anti-cancer reagent such as Mertansine and MMAE; an anti-inflammation reagent; Fluorescein such as FTIC; a peptide; a protein; a nucleotide; an oligonucleotide; a chemotherapy drug; a natural product; an immune modulator; a tubulin-binder; a DNA-alkylating agent; an HSP90 inhibitor; a DNA topoisomerase inhibitor; an anti-epigenetic agent; an HDAC inhibitor; an anti-metabolism agent; a proteasome inhibitor; a peptidomimetic; an siRNA; an antisense DNA; epothilone A, epothilone B, or paclitaxel.

In some embodiments, wherein L includes —(CH2)m-, wherein one or more CH2 are replaced by one or more groups selected from NH and C═O, m is an integer of 0-15; —(CH2)n-heteroaryl, wherein one or more CH2 are replaced by one or more groups selected from NH, C═O and —O—, n is an integer of 0-20, and the heteroaryl group has 5 to 10 ring members; —(CH2)o-C₃₋₇heterocycloalkyl-(CH2)o-CH3, wherein one or more CH2 are replaced by one or more groups selected from NH and C═O, o is an integer of 0-15; —(CH2)q-C₃₋₇cycloalkyl-(CH2)q-CH3, wherein one or more CH2 are replaced by one or more groups selected from NH and C═O, q is an integer of 0-15; —(CH2)r-C₃₋₇cycloalkyl-(CH2)r-heteroaryl, wherein one or more CH2 are replaced by one or more groups selected from NH and C═O, r is an integer of 0-15, and the heteroaryl group has 5 to 10 ring members, and said cycloalkyl, heterocycloalkyl, —CH3 and heteroaryl are optionally substituted by oxo and halogen.

In some embodiments, wherein L includes —(CH2)m-, wherein one or more CH2 are replaced by one or more groups selected from NH and C═O, m is an integer of 0-10; —(CH2)n-heteroaryl, wherein one or more CH2 are replaced by one or more groups selected from NH, C═O and —O—, n is an integer of 0-20, and the heteroaryl has

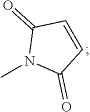

—(CH2)o-piperazinyl-(CH2)o-CH3, wherein one or more CH2 are replaced by one or more groups selected from NH and C═O, o is an integer of 0-10; —(CH2)q-cyclohexyl-(CH2)q-CH3, wherein one or more CH2 are replaced by one or more groups selected from NH and C═O, q is an integer of 0-10; —(CH2)r-cyclohexyl-(CH2)r-heteroaryl, wherein one or more CH2 are replaced by one or more groups selected from NH and C═O, r is an integer of 0-10, and the heteroaryl has

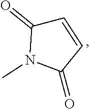

and said —CH3- is optionally substituted by halogen.

In some embodiments, wherein L includes —(CH2)m-, wherein no more than CH2 are replaced by one or more groups selected from NH and C═O, m is an integer of 0-10; —(CH2)n-heteroaryl, wherein no more than eight CH2 are replaced by one or more groups selected from NH, C═O and —O—, n is an integer of 0-18, and the heteroaryl has

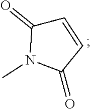

—(CH2)o-piperazinyl-(CH2)o-CH3, wherein one CH2 is replaced by one group selected from NH and C═O, o is an integer of 0-5; —(CH2)q-cyclohexyl-(CH2)q-CH3, wherein no more than six CH2 are replaced by one or more groups selected from NH and C═O, q is an integer of 0-8; —(CH2)r-cyclohexyl-(CH2)r-heteroaryl, wherein no more than five CH2 are replaced by one or more groups selected from NH and C═O, r is an integer of 0-6, and the heteroaryl has'

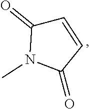

and said —CH3- is optionally substituted by Cl.

In some embodiments, wherein the compound is selected from the group consisting of:

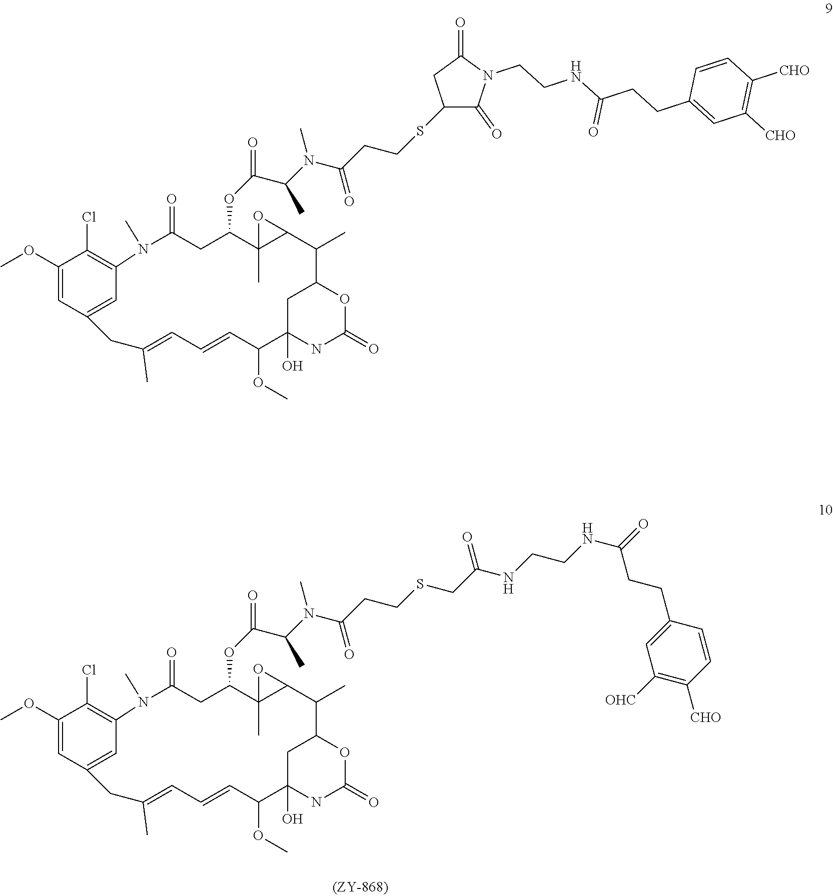 [SMILES: COc1cc2cc(c1Cl)N(C)C(=O)C[C@H](OC(=O)[C@H](C)N(C)C(=O)CCSC1CC(=O)N(CCNC(=O)CCc3ccc(C=O)c(C=O)c3)C1=O)C1(C)OC1C(C)C1CC(O)(NC(=O)O1)C(OC)/C=C/C=C(\C)C2 ; COc1cc2cc(c1Cl)N(C)C(=O)C[C@H](OC(=O)[C@H](C)N(C)C(=O)CCSCC(=O)NCCNC(=O)CCc1ccc(C=O)c(C=O)c1)C1(C)OC1C(C)C1CC(O)(NC(=O)O1)C(OC)/C=C/C=C(\C)C2]

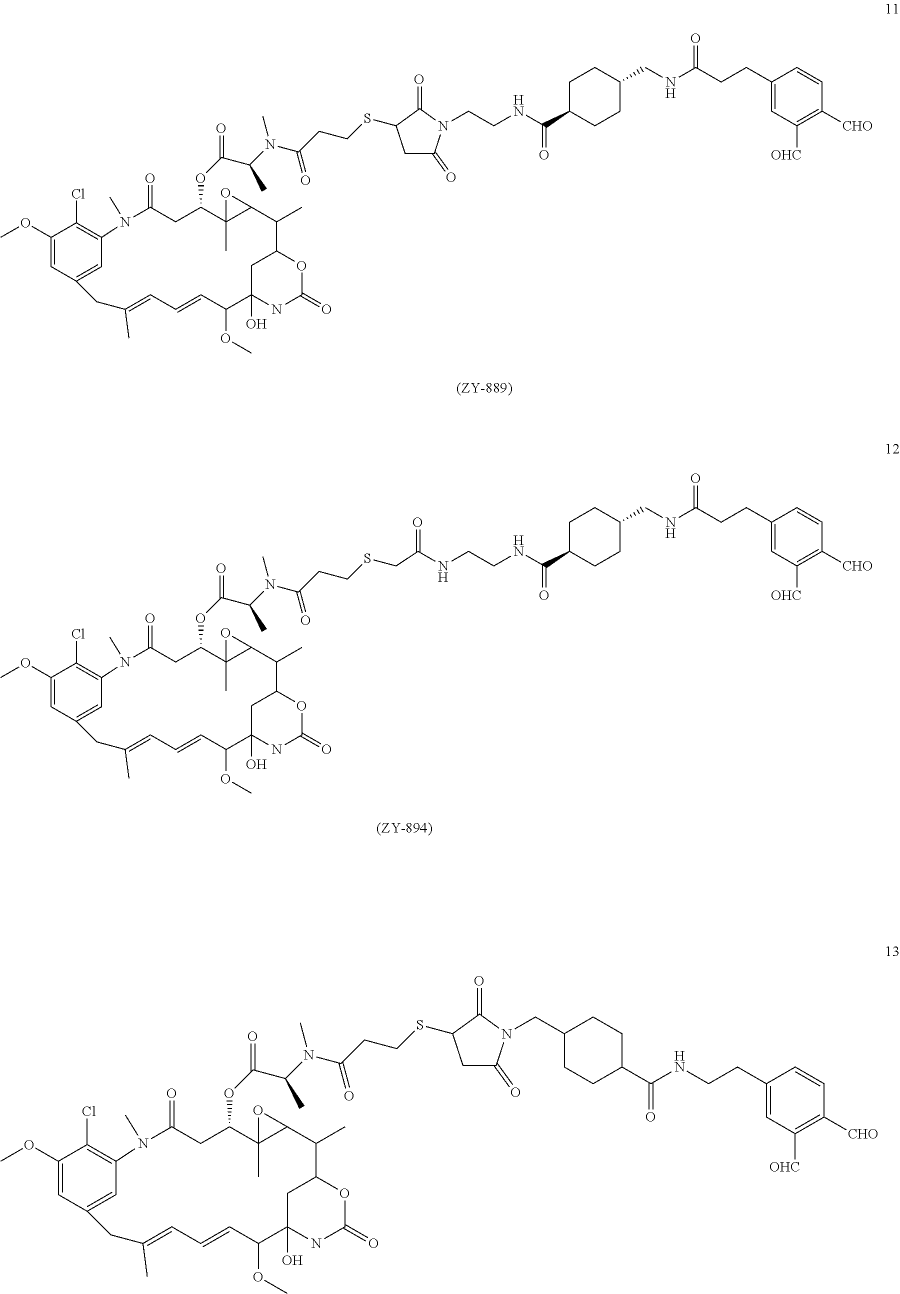

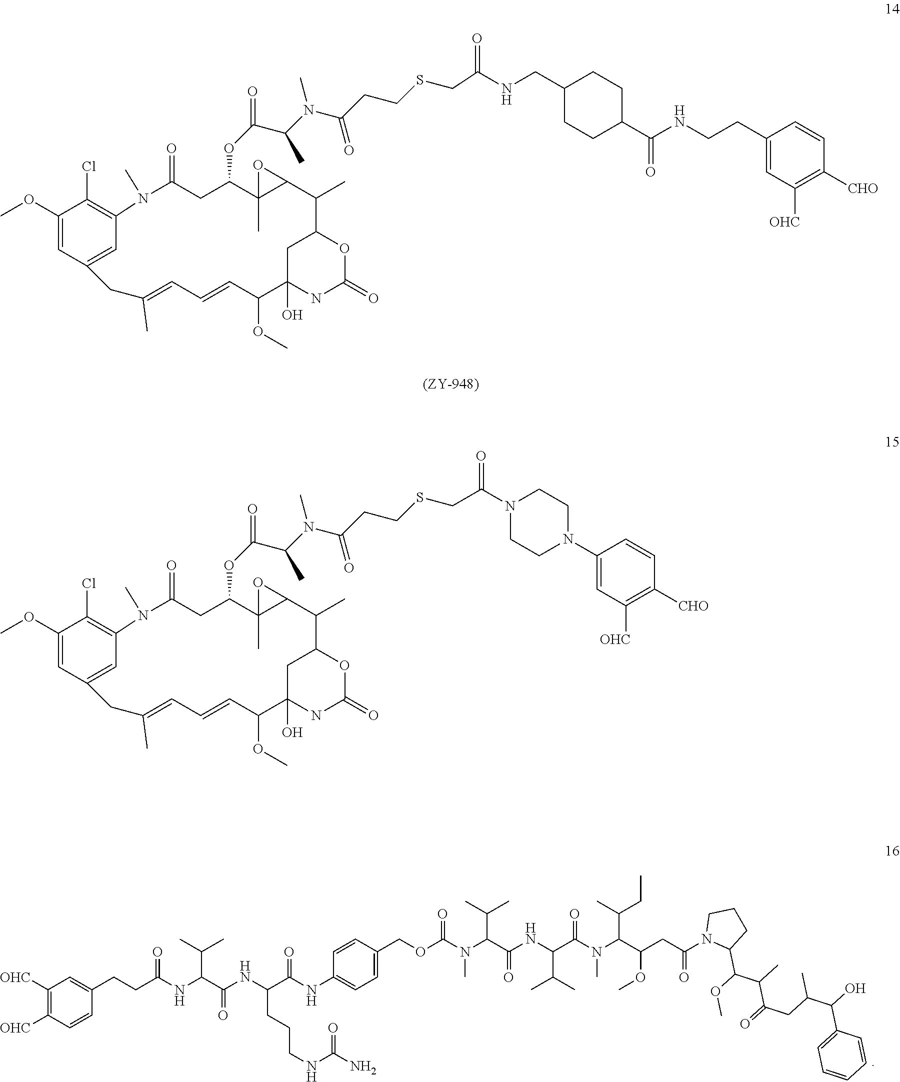 [SMILES: COc1cc2cc(c1Cl)N(C)C(=O)C[C@H](OC(=O)[C@H](C)N(C)C(=O)CCSCC(=O)NCC1CCC(C(=O)NCCc3ccc(C=O)c(C=O)c3)CC1)C1(C)OC1C(C)C1CC(O)(NC(=O)O1)C(OC)/C=C/C=C(\C)C2 ; COc1cc2cc(c1Cl)N(C)C(=O)C[C@H](OC(=O)[C@H](C)N(C)C(=O)CCSCC(=O)N1CCN(c3ccc(C=O)c(C=O)c3)CC1)C1(C)OC1C(C)C1CC(O)(NC(=O)O1)C(OC)/C=C/C=C(\C)C2 ; CCC(C)C(C(CC(=O)N1CCCC1C(OC)C(C)C(=O)CC(C)C(O)c1ccccc1)OC)N(C)C(=O)C(NC(=O)C(C(C)C)N(C)C(=O)OCc1ccc(NC(=O)C(CCCNC(N)=O)NC(=O)C(NC(=O)CCc2ccc(C=O)c(C=O)c2)C(C)C)cc1)C(C)C]

In the third aspect of the present application relates to a compound of the following formula (III): Ab-(OPA-L-D)p (III), wherein Ab is a cell binding reagent, wherein said cell binding reagent includes IgGs, bi-specific antibody, antibody fragment such as Fab, Fab′, F(ab′)2 and scFv,

Heavy-chain only antibody or Nanobody; OPA is

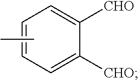

L includes alkyl, alkenyl, alkynyl, cycloalkyl, cycloalkenyl, cycloalkynyl, heterocycloalkyl, heterocycloalkenyl, heterocycloalkynyl, aryl, heteroaryl, alkylene, alkenylene, alkynylene, arylene, heteroarylene, cycloalkylene, heterocycloalkylene; —(CH2)m-, wherein one or more CH2 are replaced by one or more groups selected from NH and C═O, m is an integer of 0-20; —(CH2)n-heteroaryl, wherein one or more CH2 are replaced by one or more groups selected from NH, C═O and —O—, n is an integer of 0-20; —(CH2)o-heterocycloalkyl-(CH2)o-CH3, wherein one or more CH2 are replaced by one or more groups selected from NH and C═O, o is an integer of 0-20; —(CH2)q-cycloalkyl-(CH2)q-CH3, wherein one or more CH2 are replaced by one or more groups selected from NH and C═O, p is an integer of 0-20; —(CH2)r-cycloalkyl-(CH2)r-heteroaryl, wherein one or more CH2 are replaced by one or more groups selected from NH and C═O, r is an integer of 0-20; peptide; oligosaccharide, polyethylene glycol (PEG), and the combinations thereof, and said alkyl, alkenyl, alkynyl, cycloalkyl, cycloalkenyl, cycloalkynyl, heterocycloalkyl, heterocycloalkynyl, aryl, heteroaryl, —CH3, heterocycloalkenyl, alkylene, alkenylene, alkynylene, arylene, heteroarylene, cycloalkylene, heterocycloalkylene are optionally substituted by at least one substituents; D is independently active reagent, wherein said active reagent includes an anti-cancer reagent such as Mertansine and MMAE; an anti-inflammation reagent; Fluorescein such as FTIC; a peptide; a protein; a nucleotide; an oligonucleotide; a chemotherapy drug; a natural product; an immune modulator; a tubulin-binder; a DNA-alkylating agent; an HSP90 inhibitor; a DNA topoisomerase inhibitor; an anti-epigenetic agent; an HDAC inhibitor; an anti-metabolism agent; a proteasome inhibitor; a peptidomimetic; an siRNA; an antisense DNA; epothilone A, epothilone B, or paclitaxel; p is a integer refers to the number of active reagent attached to cell binding reagent, wherein p is an integer of 0-15.

In some embodiments, wherein L includes —(CH2)m-, wherein one or more CH2 are replaced by one or more groups selected from NH and C═O, m is an integer of 0-15; —(CH2)n-heteroaryl, wherein one or more CH2 are replaced by one or more groups selected from NH, C═O and —O—, n is an integer of 0-20, and the heteroaryl group has 5 to 10 ring members; —(CH2)o-C₃₋₇heterocycloalkyl-(CH2)o-CH3, wherein one or more CH2 are replaced by one or more groups selected from NH and C═O, o is an integer of 0-15; —(CH2)q-C₃₋₇cycloalkyl-(CH2)q-CH3, wherein one or more CH2 are replaced by one or more groups selected from NH and C═O, q is an integer of 0-15; —(CH2)r-C₃₋₇cycloalkyl-(CH2)r-heteroaryl, wherein one or more CH2 are replaced by one or more groups selected from NH and C═O, r is an integer of 0-15, and the heteroaryl group has 5 to 10 ring members, and said cycloalkyl, heterocycloalkyl, —CH3 and heteroaryl are optionally substituted by oxo and halogen.

In some embodiments, wherein L includes —(CH2)m-, wherein one or more CH2 are replaced by one or more groups selected from NH and C═O, m is an integer of 0-10; —(CH2)n-heteroaryl, wherein one or more CH2 are replaced by one or more groups selected from NH, C═O and —O—, n is an integer of 0-20, and the 5 to 10 membered heteroaryl has

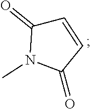

—(CH2)o-piperazinyl-(CH2)o-CH3, wherein one or more CH2 are replaced by one or more groups selected from NH and C═O, o is an integer of 0-10; —(CH2)q-cyclohexyl-(CH2)q-CH3, wherein one or more CH2 are replaced by one or more groups selected from NH and C═O, q is an integer of 0-10; —(CH2)r-cyclohexyl-(CH2)r-heteroaryl, wherein one or more CH2 are replaced by one or more groups selected from NH and C═O, r is an integer of 0-10, and the 5 to 10 membered heteroaryl has

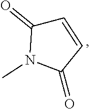

and said —CH3- is optionally substituted by halogen.

In some embodiments, wherein L includes —(CH2)m-, wherein no more than CH2 are replaced by one or more groups selected from NH and C═O, m is an integer of 0-10; —(CH2)n-heteroaryl, wherein no more than eight CH2 are replaced by one or more groups selected from NH, C═O and —O—, n is an integer of 0-18, and the 5 to 10 membered heteroaryl has

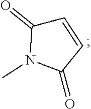

—(CH2)o-piperazinyl-(CH2)o-CH3, wherein one CH2 is replaced by one group selected from NH and C═O, o is an integer of 0-5; —(CH2)q-cyclohexyl-(CH2)q-CH3, wherein no more than six CH2 are replaced by one or more groups selected from NH and C═O, q is an integer of 0-8; —(CH2)r-cyclohexyl-(CH2)r-heteroaryl, wherein no more than five CH2 are replaced by one or more groups selected from NH and C═O, r is an integer of 0-6, and the 5 to 10 membered heteroaryl has

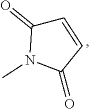

and said —CH3- is optionally substituted by Cl.

In some embodiments, wherein the OPA-L-D is selected from the group consisting of:

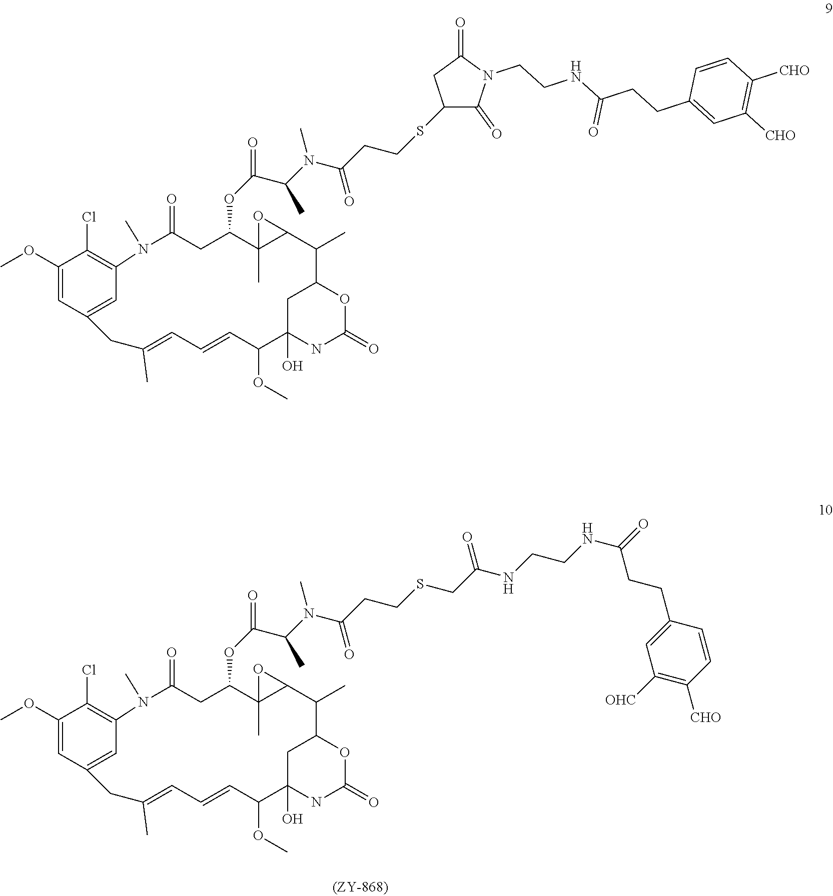 [SMILES: COc1cc2cc(c1Cl)N(C)C(=O)C[C@H](OC(=O)[C@H](C)N(C)C(=O)CCSC1CC(=O)N(CCNC(=O)CCc3ccc(C=O)c(C=O)c3)C1=O)C1(C)OC1C(C)C1CC(O)(NC(=O)O1)C(OC)/C=C/C=C(\C)C2 ; COc1cc2cc(c1Cl)N(C)C(=O)C[C@H](OC(=O)[C@H](C)N(C)C(=O)CCSCC(=O)NCCNC(=O)CCc1ccc(C=O)c(C=O)c1)C1(C)OC1C(C)C1CC(O)(NC(=O)O1)C(OC)/C=C/C=C(\C)C2]

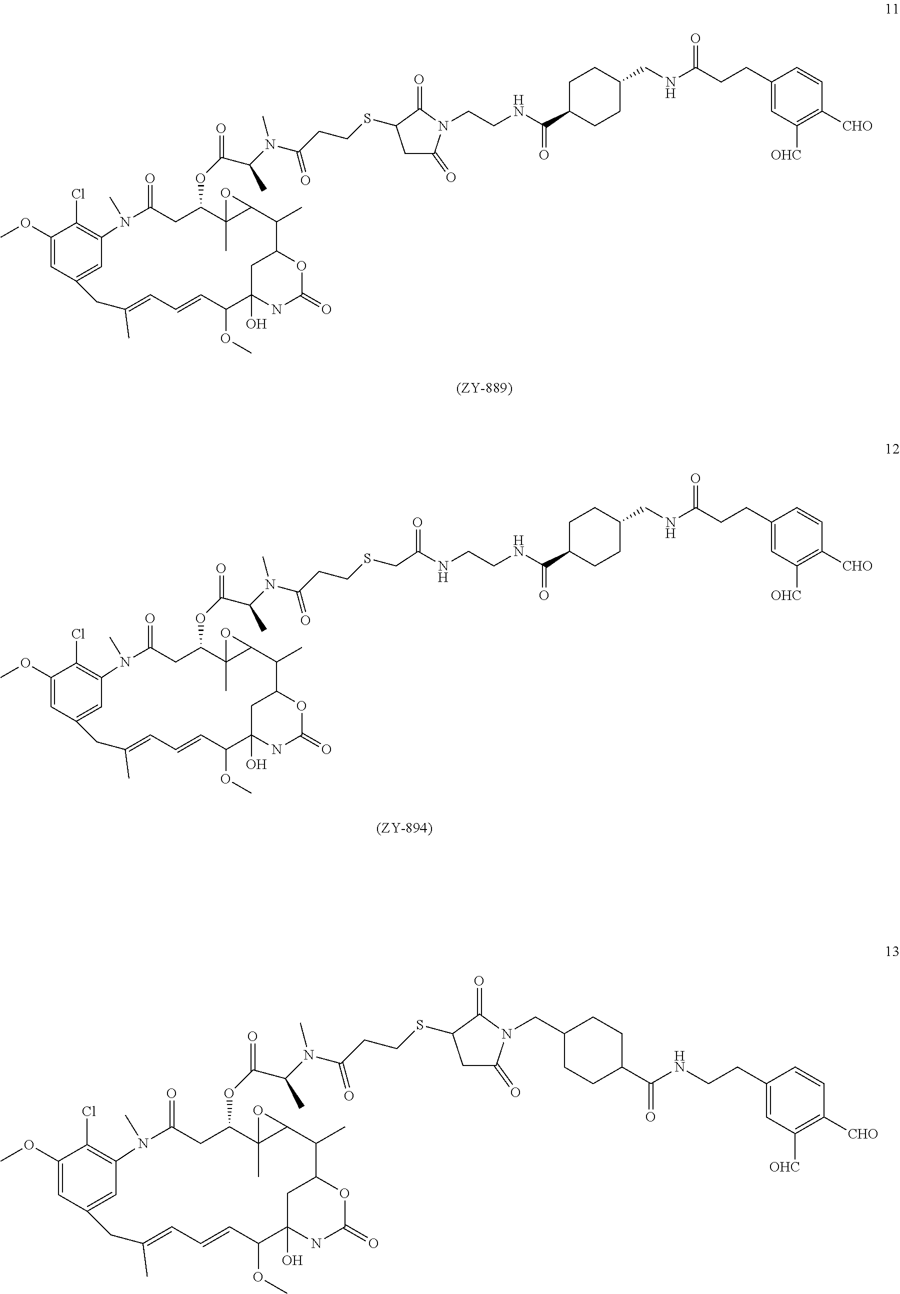

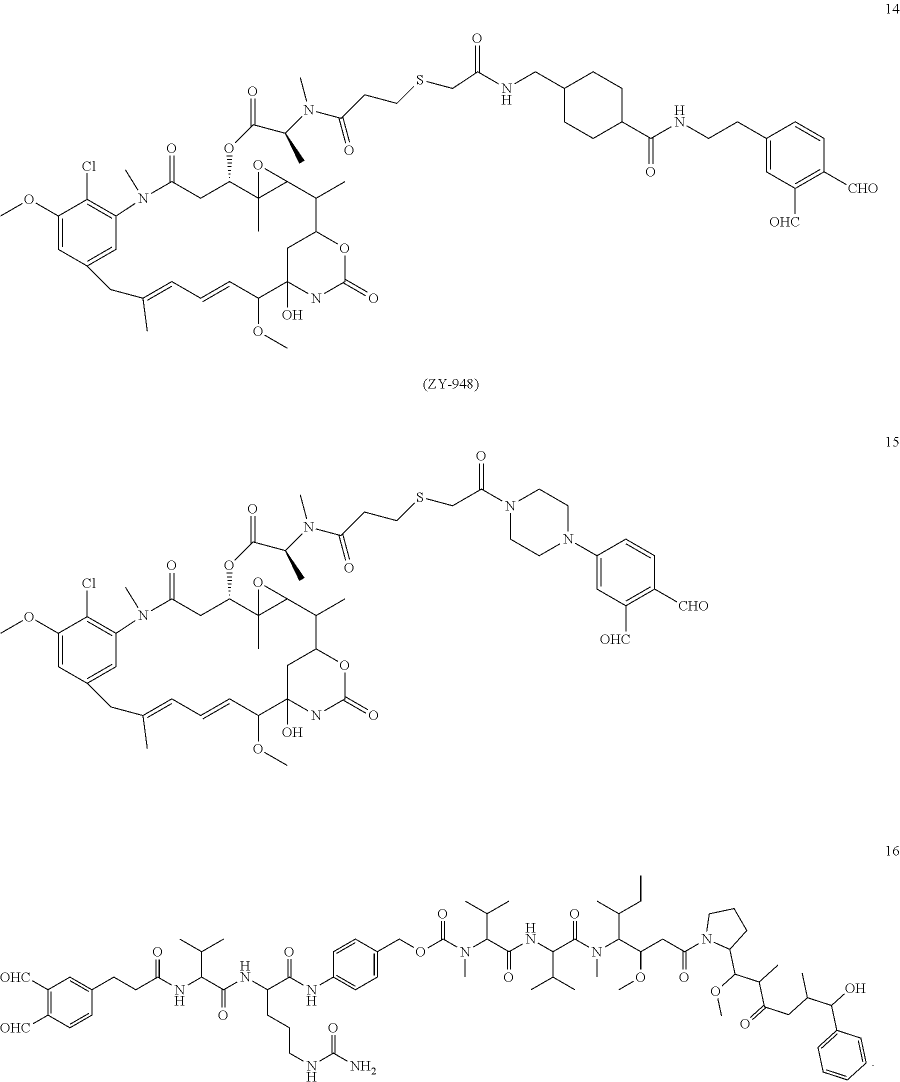 [SMILES: COc1cc2cc(c1Cl)N(C)C(=O)C[C@H](OC(=O)[C@H](C)N(C)C(=O)CCSCC(=O)NCC1CCC(C(=O)NCCc3ccc(C=O)c(C=O)c3)CC1)C1(C)OC1C(C)C1CC(O)(NC(=O)O1)C(OC)/C=C/C=C(\C)C2 ; COc1cc2cc(c1Cl)N(C)C(=O)C[C@H](OC(=O)[C@H](C)N(C)C(=O)CCSCC(=O)N1CCN(c3ccc(C=O)c(C=O)c3)CC1)C1(C)OC1C(C)C1CC(O)(NC(=O)O1)C(OC)/C=C/C=C(\C)C2 ; CCC(C)C(C(CC(=O)N1CCCC1C(OC)C(C)C(=O)CC(C)C(O)c1ccccc1)OC)N(C)C(=O)C(NC(=O)C(C(C)C)N(C)C(=O)OCc1ccc(NC(=O)C(CCCNC(N)=O)NC(=O)C(NC(=O)CCc2ccc(C=O)c(C=O)c2)C(C)C)cc1)C(C)C]

In the fourth aspect of the present application relates to a use of a compound of formula OPA-L (I) as defined above for the preparation of a conjugate of the following formula (III): Ab-(OPA-L-D)p (Ill), wherein Ab, OPA, L, D and p are defined as above.

In the fifth aspect of the present application relates to a use of a compound of formula OPA-L-D (II) as defined above for the preparation of a conjugate of the following formula (III): Ab-(OPA-L-D)p (Ill), wherein Ab, OPA, L, D and p are defined as above.

In the sixth aspect of the present application relates to a process for the preparation of conjugate of the following formula (III): Ab-(OPA-L-D)p (Ill), wherein the conjugate comprises D linked to Ab through the reaction of primary amine on Ab and OPA-L, the process comprising the steps of: (a) contacting D with OPA-L to covalently attach the OPA-L to D and therefore prepare OPA-L-D, wherein D, OPA and L are defined as above; (b) conjugating Ab to OPA-L-D by reacting the OPA-L-D with Ab to prepare the conjugate of formula (III), wherein Ab and p are defined as above; and (c) purifying the conjugate of formula (III) with down-stream steps such as buffer exchange or column purification.

### BRIEF DESCRIPTION OF THE FIGURES

FIG. 1 shows SEC-HPLC for Trastuzumab-ZY-889.

FIG. 2 shows LC-MS spectrum for Trastuzumab-ZY-889.

FIG. 3 shows SEC-HPLC for Trastuzumab-ZY-948.

FIG. 4 shows LC-MS spectrum for Trastuzumab-ZY-948.

FIG. 5 shows SEC-HPLC for HcAb-1-ZY-894.

FIG. 6 shows LC-MS spectrum for HcAb-1-ZY-894.

FIG. 7 shows SEC-HPLC for HcAb-2-ZY-894.

FIG. 8 shows LC-MS spectrum for HcAb-2-ZY-894.

### DETAILED DESCRIPTION

### Definition

As used herein, common organic abbreviations are defined as follows:

| | |
|---|---|
| ADC | Antibody drug conjugate |
| DAR | Drug to antibody ratio |
| DMA | Dimethylacetamide |
| HPLC | High performance liquid chromatography |
| SEC | Size exclusion chromatography |
| LC-MS | Liquid chromatography-mass spectrometry |
| UV-vis | UV-Visible Spectrophotometer |
| eq | Equivalents |
| TFA | Trifluoroacetic acid |
| IPA | Isopropanol |
| EDTA | Ethylene Diamine Tetraacetic Acid |

As used herein, “alkyl” refers to a straight or branched hydrocarbon chain that is fully saturated (i.e., contains no double or triple bonds). The alkyl group may have 1 to 20 carbon atoms (whenever it appears herein, a numerical range such as “1 to 20” refers to each integer in the given range; e.g., “1 to 20 carbon atoms” refers to that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 4 carbon atoms. The alkyl group may be designated as “C₁₋₄ alkyl” or similar designations. By way of example only, “C₁₋₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

As used herein, “alkoxy” refers to the formula —OR wherein R is an alkyl as is defined above, such as “C₁₋₉ alkoxy”, including but not limited to methoxy, ethoxy, n-propoxy, 1-methylethoxy (isopropoxy), n-butoxy, iso-butoxy, sec-butoxy, and tert-butoxy, and the like.

As used herein, “alkenyl” refers to a straight or branched hydrocarbon chain containing one or more double bonds. The alkenyl group may have 2 to 20 carbon atoms, although the present definition also covers the occurrence of the term “alkenyl” where no numerical range is designated. The alkenyl group may also be a medium size alkenyl having 2 to 9 carbon atoms. The alkenyl group could also be a lower alkenyl having 2 to 4 carbon atoms. The alkenyl group may be designated as “C₂₋₄ alkenyl” or similar designations. By way of example only, “C₂₋₄ alkenyl” indicates that there are two to four carbon atoms in the alkenyl chain, i.e., the alkenyl chain is selected from the group consisting of ethenyl, propen-1-yl, propen-2-yl, propen-3-yl, buten-1-yl, buten-2-yl, buten-3-yl, buten-4-yl, 1-methyl-propen-1-yl, 2-methyl-propen-1-yl, 1-ethyl-ethen-1-yl, 2-methyl-propen-3-yl, buta-1,3-dienyl, buta-1,2-dienyl, and buta-1,2-dien-4-yl. Typical alkenyl groups include, but are in no way limited to, ethenyl, propenyl, butenyl, pentenyl, and hexenyl, and the like.

As used herein, “alkynyl” refers to a straight or branched hydrocarbon chain containing one or more triple bonds. The alkynyl group may have 2 to 20 carbon atoms, although the present definition also covers the occurrence of the term “alkynyl” where no numerical range is designated. The alkynyl group may also be a medium size alkynyl having 2 to 9 carbon atoms. The alkynyl group could also be a lower alkynyl having 2 to 4 carbon atoms. The alkynyl group may be designated as “C₂₋₄ alkynyl” or similar designations. By way of example only, “C₂₋₄ alkynyl” indicates that there are two to four carbon atoms in the alkynyl chain, i.e., the alkynyl chain is selected from the group consisting of ethynyl, propyn-1-yl, propyn-2-yl, butyn-1-yl, butyn-3-yl, butyn-4-yl, and 2-butynyl. Typical alkynyl groups include, but are in no way limited to, ethynyl, propynyl, butynyl, pentynyl, and hexynyl, and the like.

As used herein, “alkylene” refers to divalent groups, for example, “(C₁-C₁₂)alkylene”, including methylene (i.e., —CH2-), ethylene, n-propylene, isopropylene, t-butylene, pentylene, hexylene, octylene, nonylene, decylene, undecylene, dodecylene and the like. With alternative common name, deriving from the name of the corresponding alkanes, the above divalent groups can be referred to also as methanediyl, ethanediyl, n-propanediyl, propan-1,2-diyl and the like.

As used herein, “alkenylene” refers to divalent groups, for example, “(C₂-C₁₂)alkenylene”, including ethenylene, propenylene, butenylene, pentenylene, hexenylene, heptenylene, octenylene, nonenylene, decenylene, undecenylene, dodecenylene and the like.

As used herein, “alkynylene” refers to divalent groups, for example, “(C₂—C)alkynylene”, including ethynylene, propynylene, butynylene, pentynylene, hexynylene and the like; otherwise commonly referred to as ethynediyl, propynediyl, butyndiyl and the like.

As used herein, “aromatic” refers to a ring or ring system having a conjugated pi electron system and includes both carbocyclic aromatic (e.g., phenyl) and heterocyclic aromatic groups (e.g., pyridine). The term includes monocyclic or fused-ring polycyclic (i.e., rings which share adjacent pairs of atoms) groups provided that the entire ring system is aromatic.

As used herein, “aryl” refers to an unsaturated aromatic carbocyclic group of from 6 to 14 carbon atoms having a single ring (e.g. phenyl) or multiple condensed rings (e.g. naphthyl). Illustrative aryl groups include phenyl.

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. In some embodiments, the heteroaryl group has 5 to 10 ring members or 5 to 7 ring members. The heteroaryl group may be designated as “5-7 membered heteroaryl,” “5-10 membered heteroaryl,” or similar designations. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinlinyl, benzimidazolyl, benzoxazolyl, benzothiazolyl, indolyl, isoindolyl, and benzothienyl.

As used herein, “arylene” and “heteroarylene” refer to divalent groups, for example, a phenylene, biphenylene and thienylene.

As used herein, “carbocyclyl” refers to a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “C₃₋₆ carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle[2.2.2]octanyl, adamantyl, and spiro[4.4]nonanyl.

As used herein, “cycloalkyl” refers to a fully saturated carbocyclyl ring or ring system. The term “C₃₋₇ cycloalkyl” as used herein refers to a fully saturated carbocyclyl ring or ring system with 3 to 7 carbon atoms. Suitable C₃₋₇cycloalkyl groups may comprise benzo-fused analogues thereof. Illustrative C₃₋₇ cycloalkyl groups include cyclopropyl, cyclobutyl, benzocyclobutenyl, cyclopentyl, indanyl, cyclohexyl and cycloheptyl.

As used herein, “cycloalkenyl” refers to a carbocyclyl ring or ring system having at least one double bond, wherein no ring in the ring system is aromatic. An example is cyclohexenyl.

As used herein, “cycloalkynyl” refers to a carbocyclyl ring or ring system having at least one triple bond, wherein no ring in the ring system is aromatic.

As used herein, “heterocyclyl” refers to a non-aromatic cyclic ring or ring system containing at least one heteroatom in the ring backbone. Heterocyclyls may be joined together in a fused, bridged or spiro-connected fashion. Heterocyclyls may have any degree of saturation provided that at least one ring in the ring system is not aromatic. Thus, heterocarbocyclyls include heterocycloalkyls, heterocycloalkenyls, and heterocycloalkynyls. The heteroatom(s) may be present in either a non-aromatic or aromatic ring in the ring system. The heterocyclyl group may have 3 to 20 ring members (i.e., the number of atoms making up the ring backbone, including carbon atoms and heteroatoms), although the present definition also covers the occurrence of the term “heterocyclyl” where no numerical range is designated. The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations. In preferred six membered monocyclic heterocyclyls, the heteroatom(s) are selected from one up to three of O (oxygen), N (nitrogen) or S (sulfur), and in preferred five membered monocyclic heterocyclyls, the heteroatom(s) are selected from one or two heteroatoms selected from O (oxygen), N (nitrogen), or S (sulfur).

As used herein, “heterocycloalkyl” refers to saturated cyclic rings and at least one heteroatom selected from oxygen, sulphur and nitrogen, and may comprise benzo-fused analogues thereof, for example, C₃₋₇ heterocycloalkyl. The term “C₃₋₇ heterocycloalkyl” as used herein refers to saturated monocyclic rings containing 3 to 7 carbon atoms and at least one heteroatom selected from oxygen, sulphur and nitrogen, and may comprise benzo-fused analogues thereof. Illustrative heterocycloalkyl groups include oxetanyl, azetidinyl, tetrahydrofuranyl, dihydrobenzo-furanyl, dihydrobenzothienyl, pyrrolidinyl, indolinyl, dihydroisoindolinyl, isoindolinyl, oxazolidinyl, thiazolidinyl, isothiazolidinyl, imidazolidinyl, tetrahydropyranyl, chromanyl, tetrahydro-thiopyranyl, piperidinyl, 1,2,3,4-tetrahydroquinolinyl, 1,2,3,4-tetrahydroisoquinolinyl, piperazinyl, 1,2,3,4-tetrahydroquinoxalinyl, hexahydro-[1,2,5]thiadiazolo[2,3-a]pyrazinyl, homopiperazinyl, morpholinyl, benzoxazinyl, thiomorpholinyl, azepanyl, oxazepanyl, diazepanyl, thiadiazepanyl, azocanyl, (imino)(oxo)thiazinanyl, (oxo)thiazinanyl, (dioxo)thiazinanyl, tetrahydrothiophenyl, (oxo)tetrahydrothiophenyl, (dioxo)tetrahydrothiophenyl and (oxo)thiomorpholinyl.

As used herein, “heterocycloalkenyl” refers to monounsaturated or polyunsaturated monocyclic rings and at least one heteroatom selected from oxygen, sulphur and nitrogen, and may comprise benzo-fused analogues thereof, for example, C₃₋₇ heterocycloalkenyl. The term “C₃₋₇ heterocycloalkenyl” as used herein refers to monounsaturated or polyunsaturated monocyclic rings containing 3 to 7 carbon atoms and at least one heteroatom selected from oxygen, sulphur and nitrogen, and may comprise benzo-fused analogues thereof. Illustrative heterocycloalkenyl groups include thiazolinyl, imidazolinyl, dihydropyranyl, dihydrothiopyranyl, 1,2,3,6-tetrahydropyridinyl, 1,2-dihydropyridinyl and 1,2-dihydropyrimidinyl.

As used herein, “cycloalkylene” and “heterocycloalkylene” herewith refer to divalent groups, wherein “cycloalkylene” refers to saturated cycloalkane-diyl and partially saturated monocyclic groups such as cycloalkene-diyl, while “heterocycloalkylene” refers to cycloalkylene as defined above and at least one heteroatom selected from oxygen, sulphur and nitrogen, for example “(C₃-C₅)cycloalkylene” and “(C₃-C₈)heterocycloalkylene”, including cyclopropylene, cyclobutylene, cyclopentylene, cyclohexylene, cycloheptylene, bicyclo[2.2.1]hept-2-ylene and quinuclidinylene, pyrrolidinylene, piperidinylene, azabicyclo[3.2.1]octan-3-ylene, azoniabicyclo[2.2.2]octanylene, [1.2.3.6]tetrahydropyridin-[1.4]diyl and the like.

As used herein, an oxo moiety is represented by (0) as an alternative to other common representations, e.g. (═O).

As used herein, “halogen” refers to fluorine, chlorine, bromine and iodine atoms, typically fluorine, chlorine or bromine atoms.

In the first aspect of the present application relates to a compound of the following formula (I): OPA-L (O), wherein OPA is

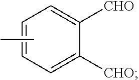

L includes alkyl, alkenyl, alkynyl, cycloalkyl, cycloalkenyl, cycloalkynyl, heterocycloalkyl, heterocycloalkenyl, heterocycloalkynyl, aryl, heteroaryl, alkylene, alkenylene, alkynylene, arylene, heteroarylene, cycloalkylene, heterocycloalkylene; —(CH2)m-, wherein one or more CH2 are replaced by one or more groups selected from NH and C═O, m is an integer of 0-20 (including 0, 1, 2, 3, 4, 5, 6, 7, 8, 9, 10, 11, 12, 13, 14, 15, 16, 17, 18, 19 or 20, and the range between said integers); —(CH2)n-heteroaryl, wherein one or more CH2 are replaced by one or more groups selected from —NH, —C═O and —O—, n is an integer of 0-20 (including 0, 1, 2, 3, 4, 5, 6, 7, 8, 9, 10, 11, 12, 13, 14, 15, 16, 17, 18, 19 or 20, and the range between said integers); —(CH2)o-heterocycloalkyl-(CH2)o-CH3, wherein one or more CH2 are replaced by one or more groups selected from NH and C═O, o is an integer of 0-20; —(CH2)q-cycloalkyl-(CH2)q-CH3, wherein one or more CH2 are replaced by one or more groups selected from NH and C═O, q is an integer of 0-20 (including 0, 1, 2, 3, 4, 5, 6, 7, 8, 9, 10, 11, 12, 13, 14, 15, 16, 17, 18, 19 or 20, and the range between said integers); —(CH2)r-cycloalkyl-(CH2)r-heteroaryl, wherein one or more CH2 are replaced by one or more groups selected from NH and C═O, r is an integer of 0-20 (including 0, 1, 2, 3, 4, 5, 6, 7, 8, 9, 10, 11, 12, 13, 14, 15, 16, 17, 18, 19 or 20, and the range between said integers); peptide like di-peptides, tri-peptides, tetra-peptide, penta-peptide; oligosaccharide, polyethylene glycol (PEG), and the combinations thereof, and said alkyl, alkenyl, alkynyl, cycloalkyl, cycloalkenyl, cycloalkynyl, heterocycloalkyl, heterocycloalkynyl, aryl, heteroaryl, —CH3, heterocycloalkenyl, alkylene, alkenylene, alkynylene, arylene, heteroarylene, cycloalkylene, heterocycloalkylene are optionally substituted by at least one substituents.

In some embodiments, said substituents includes but not limits to halogen, halo(C₁₋₆)alkyl, C₁₋₆ alkyl, (C₃₋₇)cycloalkyl, C₂₋₆ alkenyl, C₁₋₆ alkoxy, (C₁₋₆)alkoxy(C₁₋₆)alkyl, amino-(C₁₋₆)alkyl, C₁_6 alkylamino, di(C₁₋₆)alkylamino, (C₁₋₆)alkoxy(C₁₋₆)alkylamino, formyl, acetyl, C₂₋₆ alkylcarbonyl, (C₂₋₆)alkyl-carbonyloxy(C₁₋₆)alkyl, C₂₋₆ alkoxycarbonyl, C₂₋₆ alkoxycarbonyl(C₁₋₆)alkyl, (C₁₋₆)alkoxyamino, aminocarbonyl or amido.

In some embodiments, L includes —(CH2)m-, wherein one or more CH2 are replaced by one or more groups selected from NH and C═O, m is an integer of 0-15 (including 0, 1, 2, 3, 4, 5, 6, 7, 8, 9, 10, 11, 12, 13, 14 or 15, and the range between said integers); —(CH2)n-heteroaryl, wherein one or more CH2 are replaced by one or more groups selected from NH, C═O and —O—, n is an integer of 0-20 (including 0, 1, 2, 3, 4, 5, 6, 7, 8, 9, 10, 11, 12, 13, 14, 15, 16, 17, 18, 19 or 20, and the range between said integers), and the heteroaryl group has 5 to 10 ring members; —(CH2)o-C₃₋₇heterocycloalkyl-(CH2)o-CH3, wherein one or more CH2 are replaced by one or more groups selected from NH and C═O, o is an integer of 0-15 (including 0, 1, 2, 3, 4, 5, 6, 7, 8, 9, 10, 11, 12, 13, 14 or 15, and the range between said integers); —(CH2)q-C₃₋₇cycloalkyl-(CH2)q-CH3, wherein one or more CH2 are replaced by one or more groups selected from NH and C═O, q is an integer of 0-15 (including 0, 1, 2, 3, 4, 5, 6, 7, 8, 9, 10, 11, 12, 13, 14 or 15, and the range between said integers); —(CH2)r-C₃₋₇cycloalkyl-(CH2)r-heteroaryl, wherein one or more CH2 are replaced by one or more groups selected from NH and C═O, r is an integer of 0-15 (including 0, 1, 2, 3, 4, 5, 6, 7, 8, 9, 10, 11, 12, 13, 14 or 15, and the range between said integers), and the heteroaryl group has 5 to 10 ring members, and said cycloalkyl, heterocycloalkyl, —CH3 and heteroaryl are optionally substituted by oxo and halogen.

In some embodiments, wherein L includes —(CH2)m-, wherein one or more CH2 are replaced by one or more groups selected from NH and C═O, m is an integer of 0-10 (including 0, 1, 2, 3, 4, 5, 6, 7, 8, 9 or 10, and the range between said integers); —(CH2)n-heteroaryl, wherein one or more CH2 are replaced by one or more groups selected from NH, C═O and —O—, n is an integer of 0-20 (including 0, 1, 2, 3, 4, 5, 6, 7, 8, 9, 10, 11, 12, 13, 14, 15, 16, 17, 18, 19 or 20, and the range between said integers), and the heteroaryl has

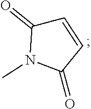

—(CH2)o-piperazinyl-(CH2)o-CH3, wherein one or more CH2 are replaced by one or more groups selected from NH and C═O, o is an integer of 0-10 (including 0, 1, 2, 3, 4, 5, 6, 7, 8, 9 or 10, and the range between said integers); —(CH2)q-cyclohexyl-(CH2)q-CH3, wherein one or more CH2 are replaced by one or more groups selected from NH and C═O, q is an integer of 0-10; —(CH2)r-cyclohexyl-(CH2)r-heteroaryl, wherein one or more CH2 are replaced by one or more groups selected from NH and C═O, r is an integer of 0-10 (including 0, 1, 2, 3, 4, 5, 6, 7, 8, 9 or 10, and the range between said integers), and the heteroaryl has

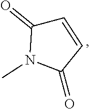

and said —CH3- is optionally substituted by halogen.

In some embodiments, wherein L includes —(CH2)m-, wherein no more than CH2 are replaced by one or more groups selected from NH and C═O, m is an integer of 0-10 (including 0, 1, 2, 3, 4, 5, 6, 7, 8, 9 or 10, and the range between said integers); —(CH2)n-heteroaryl, wherein no more than eight CH2 are replaced by one or more groups selected from NH, C═O and —O—, n is an integer of 0-18 (including 0, 1, 2, 3, 4, 5, 6, 7, 8, 9, 10, 11, 12, 13, 14, 15, 16, 17 or 18, and the range between said integers), and the heteroaryl has

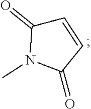

—(CH2)o-piperazinyl-(CH2)o-CH3, wherein one CH2 is replaced by one group selected from NH and C═O, o is an integer of 0-5 (including 0, 1, 2, 3, 4 or 5, and the range between said integers); —(CH2)q-cyclohexyl-(CH2)q-CH3, wherein no more than six CH2 are replaced by one or more groups selected from NH and C═O, q is an integer of 0-8 (including 0, 1, 2, 3, 4, 5, 6, 7 or 8, and the range between said integers); —(CH2)r-cyclohexyl-(CH2)r-heteroaryl, wherein no more than five CH2 are replaced by one or more groups selected from NH and C═O, r is an integer of 0-6 (including 0, 1, 2, 3, 4, 5 or 6, and the range between said integers), and the heteroaryl has

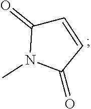

and said —CH3- is optionally substituted by Cl.

In some embodiments, wherein the compound is selected from the group consisting of:

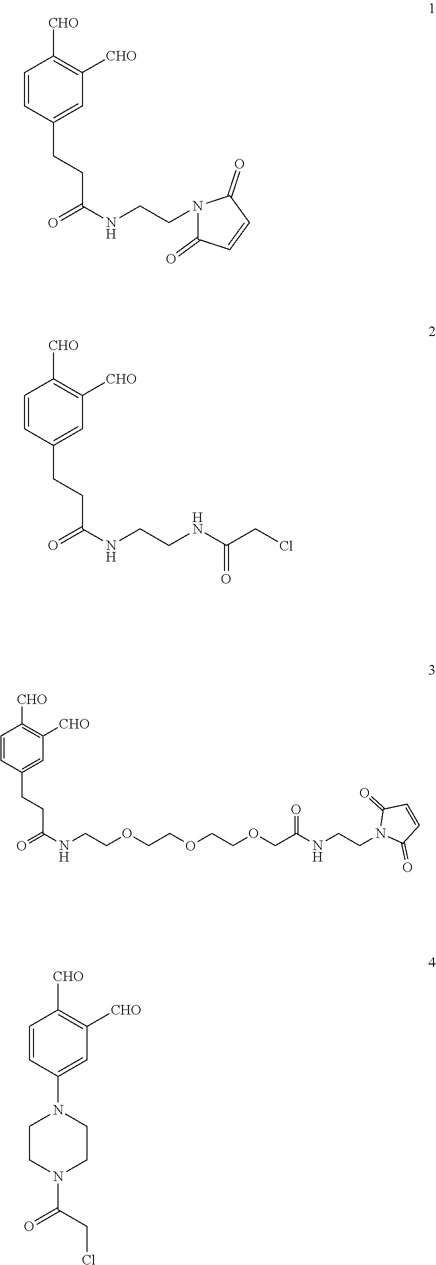 [SMILES: O=Cc1ccc(CCC(=O)NCCN2C(=O)C=CC2=O)cc1C=O ; O=Cc1ccc(CCC(=O)NCCNC(=O)CCl)cc1C=O ; O=Cc1ccc(CCC(=O)NCCOCCOCCOCC(=O)NCCN2C(=O)C=CC2=O)cc1C=O ; O=Cc1ccc(N2CCN(C(=O)CCl)CC2)cc1C=O]

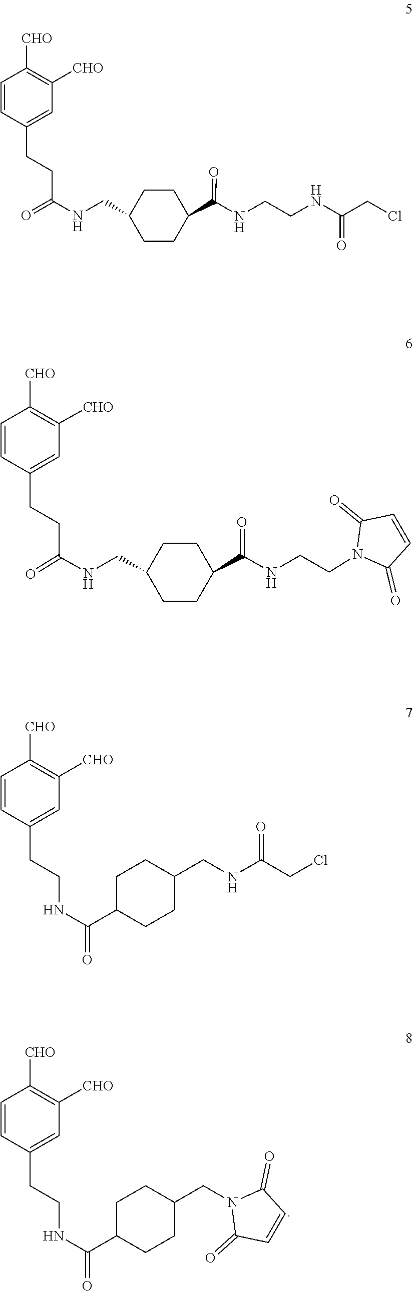 [SMILES: O=Cc1ccc(CCC(=O)NC[C@H]2CC[C@H](C(=O)NCCNC(=O)CCl)CC2)cc1C=O ; O=Cc1ccc(CCC(=O)NC[C@H]2CC[C@H](C(=O)NCCN3C(=O)C=CC3=O)CC2)cc1C=O ; O=Cc1ccc(CCNC(=O)C2CCC(CNC(=O)CCl)CC2)cc1C=O ; O=Cc1ccc(CCNC(=O)C2CCC(CN3C(=O)C=CC3=O)CC2)cc1C=O]

In the second aspect of the present application relates to a compound of the following formula (II): OPA-L-D (II), wherein OPA is

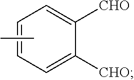

L includes alkyl, alkenyl, alkynyl, cycloalkyl, cycloalkenyl, cycloalkynyl, heterocycloalkyl, heterocycloalkenyl, heterocycloalkynyl, aryl, heteroaryl, alkylene, alkenylene, alkynylene, arylene, heteroarylene, cycloalkylene, heterocycloalkylene; —(CH2)m-, wherein one or more CH2 are replaced by one or more groups selected from NH and C═O, m is an integer of 0-20 (including 0, 1, 2, 3, 4, 5, 6, 7, 8, 9, 10, 11, 12, 13, 14, 15, 16, 17, 18, 19 or 20, and the range between said integers); —(CH2)n-heteroaryl, wherein one or more CH2 are replaced by one or more groups selected from NH, C═O and —O—, n is an integer of 0-20 (including 0, 1, 2, 3, 4, 5, 6, 7, 8, 9, 10, 11, 12, 13, 14, 15, 16, 17, 18, 19 or 20, and the range between said integers); —(CH2)o-heterocycloalkyl-(CH2)o-CH3, wherein one or more CH2 are replaced by one or more groups selected from NH and C═O, o is an integer of 0-20 (including 0, 1, 2, 3, 4, 5, 6, 7, 8, 9, 10, 11, 12, 13, 14, 15, 16, 17, 18, 19 or 20, and the range between said integers); —(CH2)q-cycloalkyl-(CH2)q-CH3, wherein one or more CH2 are replaced by one or more groups selected from NH and C═O, q is an integer of 0-20 (including 0, 1, 2, 3, 4, 5, 6, 7, 8, 9, 10, 11, 12, 13, 14, 15, 16, 17, 18, 19 or 20, and the range between said integers); —(CH2)r-cycloalkyl-(CH2)r-heteroaryl, wherein one or more CH2 are replaced by one or more groups selected from NH and C═O, r is an integer of 0-20 (including 0, 1, 2, 3, 4, 5, 6, 7, 8, 9, 10, 11, 12, 13, 14, 15, 16, 17, 18, 19 or 20, and the range between said integers); peptide; oligosaccharide, polyethylene glycol (PEG), and the combinations thereof, and said alkyl, alkenyl, alkynyl, cycloalkyl, cycloalkenyl, cycloalkynyl, heterocycloalkyl, heterocycloalkynyl, aryl, heteroaryl, —CH3, heterocycloalkenyl, alkylene, alkenylene, alkynylene, arylene, heteroarylene, cycloalkylene, heterocycloalkylene are optionally substituted by at least one substituents; D is independently active reagent, wherein said active reagent includes an anti-cancer reagent such as Mertansine and MMAE; an anti-inflammation reagent; Fluorescein such as FTIC; a peptide; a protein; a nucleotide; an oligonucleotide; a chemotherapy drug; a natural product; an immune modulator; a tubulin-binder; a DNA-alkylating agent; an HSP90 inhibitor; a DNA topoisomerase inhibitor; an anti-epigenetic agent; an HDAC inhibitor; an anti-metabolism agent; a proteasome inhibitor; a peptidomimetic; an siRNA; an antisense DNA; epothilone A, epothilone B, or paclitaxel.

In some embodiments, said substituents includes but not limits to halogen, halo(C₁₋₆)alkyl, C₁₋₆ alkyl, (C₃₋₇)cycloalkyl, C₂₋₆ alkenyl, C₁₋₆ alkoxy, (C₁₋₆)alkoxy(C₁₋₆)alkyl, amino-(C₁₋₆)alkyl, C₁_6 alkylamino, di(C₁₋₆)alkylamino, (C₁₋₆)alkoxy(C₁₋₆)alkylamino, formyl, acetyl, C₂₋₆ alkylcarbonyl, (C₂₋₆)alkyl-carbonyloxy(C₁₋₆)alkyl, C₂₋₆ alkoxycarbonyl, C₂₋₆ alkoxycarbonyl(C₁₋₆)alkyl, (C₁₋₆)alkoxyamino, aminocarbonyl or amido.

In some embodiments, D is independently active reagent, wherein said active reagent is selected from a group consisting of PBD dimer, Camptothecin Derivatives, Doxorubicin Derivatives, Calicheamicin, Duocarmycin Derivatives, Amanitin, Tubulysin Derivatives, Diphtheria toxin, Azonafide, Budesonide, Dasatinib, Desacetylvinblastine hydrazide, Dexamethasone, Hemiasterlin Analogue, Eribulin, FK506, Na, K-ATP inhibitor, Nigrin B, Phthalocyanine dye, PM050489, Rifalogue, Steroidal glycoside, Thienoindole, Yttrium-90.

In some embodiments, wherein L includes —(CH2)m-, wherein one or more CH2 are replaced by one or more groups selected from NH and C═O, m is an integer of 0-15 (including 0, 1, 2, 3, 4, 5, 6, 7, 8, 9, 10, 11, 12, 13, 14 or 15, and the range between said integers); —(CH2)n-heteroaryl, wherein one or more CH2 are replaced by one or more groups selected from NH, C═O and —O—, n is an integer of 0-20 (including 0, 1, 2, 3, 4, 5, 6, 7, 8, 9, 10, 11, 12, 13, 14, 15, 16, 17, 18, 19 or 20, and the range between said integers), and the heteroaryl group has 5 to 10 ring members; —(CH2)o-C₃₋₇heterocycloalkyl-(CH2)o-CH3, wherein one or more CH2 are replaced by one or more groups selected from NH and C═O, o is an integer of 0-15; —(CH2)q-C₃₋₇cycloalkyl-(CH2)q-CH3, wherein one or more CH2 are replaced by one or more groups selected from NH and C═O, q is an integer of 0-15 (including 0, 1, 2, 3, 4, 5, 6, 7, 8, 9, 10, 11, 12, 13, 14 or 15, and the range between said integers); —(CH2)r-C₃₋₇cycloalkyl-(CH2)r-heteroaryl, wherein one or more CH2 are replaced by one or more groups selected from NH and C═O, r is an integer of 0-15 (including 0, 1, 2, 3, 4, 5, 6, 7, 8, 9, 10, 11, 12, 13, 14 or 15, and the range between said integers), and the heteroaryl group has 5 to 10 ring members, and said cycloalkyl, heterocycloalkyl, —CH3 and heteroaryl are optionally substituted by oxo and halogen.

In some embodiments, wherein L includes —(CH2)m-, wherein one or more CH2 are replaced by one or more groups selected from NH and C═O, m is an integer of 0-10 (including 0, 1, 2, 3, 4, 5, 6, 7, 8, 9 or 10, and the range between said integers); —(CH2)n-heteroaryl, wherein one or more CH2 are replaced by one or more groups selected from NH, C═O and —O—, n is an integer of 0-20, and the heteroaryl has

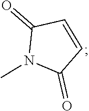

—(CH2)o-piperazinyl-(CH2)o-CH3, wherein one or more CH2 are replaced by one or more groups selected from NH and C═O, o is an integer of 0-10 (including 0, 1, 2, 3, 4, 5, 6, 7, 8, 9 or 10, and the range between said integers); —(CH2)q-cyclohexyl-(CH2)q-CH3, wherein one or more CH2 are replaced by one or more groups selected from NH and C═O, q is an integer of 0-10 (including 0, 1, 2, 3, 4, 5, 6, 7, 8, 9 or 10, and the range between said integers); —(CH2)r-cyclohexyl-(CH2)r-heteroaryl, wherein one or more CH2 are replaced by one or more groups selected from NH and C═O, r is an integer of 0-10 (including 0, 1, 2, 3, 4, 5, 6, 7, 8, 9 or 10, and the range between said integers), and the heteroaryl has

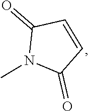

and said —CH3- is optionally substituted by halogen.

In some embodiments, wherein L includes —(CH2)m-, wherein no more than CH2 are replaced by one or more groups selected from NH and C═O, m is an integer of 0-10 (including 0, 1, 2, 3, 4, 5, 6, 7, 8, 9 or 10, and the range between said integers); —(CH2)n-heteroaryl, wherein no more than eight CH2 are replaced by one or more groups selected from NH, C═O and —O—, n is an integer of 0-18 (including 0, 1, 2, 3, 4, 5, 6, 7, 8, 9, 10, 11, 12, 13, 14, 15, 16, 17 or 18, and the range between said integers), and the heteroaryl has

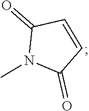

—(CH2)o-piperazinyl-(CH2)o-CH3, wherein one CH2 is replaced by one group selected from NH and C═O, o is an integer of 0-5 (including 0, 1, 2, 3, 4 or 5, and the range between said integers); —(CH2)q-cyclohexyl-(CH2)q-CH3, wherein no more than six CH2 are replaced by one or more groups selected from NH and C═O, q is an integer of 0-8 (including 0, 1, 2, 3, 4, 5, 6, 7 or 8, and the range between said integers); —(CH2)r-cyclohexyl-(CH2)r-heteroaryl, wherein no more than five CH2 are replaced by one or more groups selected from NH and C═O, r is an integer of 0-6 (including 0, 1, 2, 3, 4, 5 or 6, and the range between said integers), and the heteroaryl has

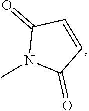

and said —CH3- is optionally substituted by Cl.

In some embodiments, wherein the compound is selected from the group consisting of:

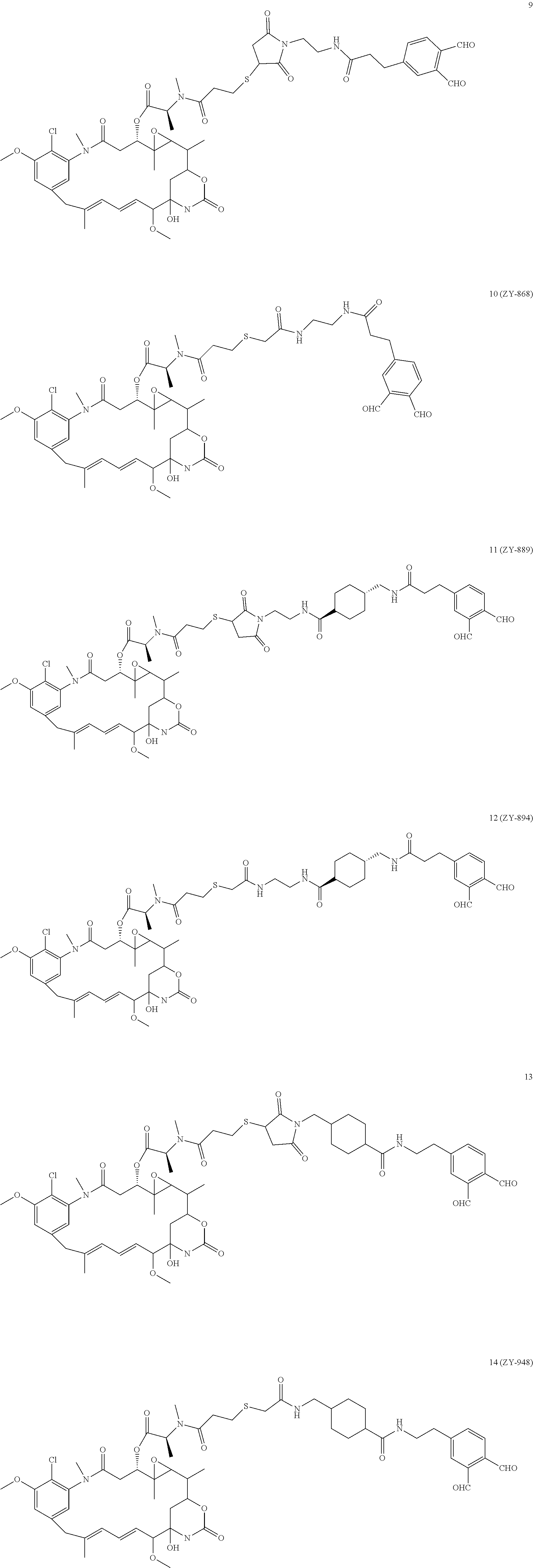

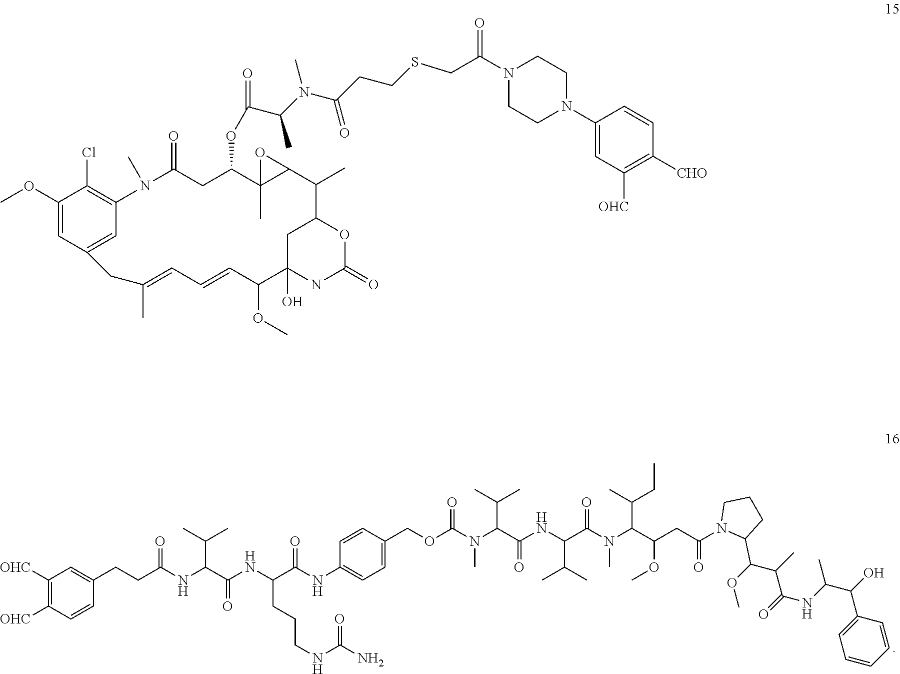 [SMILES: COc1cc2cc(c1Cl)N(C)C(=O)C[C@H](OC(=O)[C@H](C)N(C)C(=O)CCSCC(=O)N1CCN(c3ccc(C=O)c(C=O)c3)CC1)C1(C)OC1C(C)C1CC(O)(NC(=O)O1)C(OC)/C=C/C=C(\C)C2 ; CCC(C)C(C(CC(=O)N1CCCC1C(OC)C(C)C(=O)NC(C)C(O)c1ccccc1)OC)N(C)C(=O)C(NC(=O)C(C(C)C)N(C)C(=O)OCc1ccc(NC(=O)C(CCCNC(N)=O)NC(=O)C(NC(=O)CCc2ccc(C=O)c(C=O)c2)C(C)C)cc1)C(C)C]

In the third aspect of the present application relates to a compound of the following formula (III): Ab-(OPA-L-D)p (111), wherein Ab is a cell binding reagent, wherein said cell binding reagent includes IgGs, bi-specific antibody, antibody fragment such as Fab, Fab′, F(ab′)2 and scFv, Heavy-chain only antibody or Nanobody; OPA is

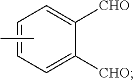

L includes alkyl, alkenyl, alkynyl, cycloalkyl, cycloalkenyl, cycloalkynyl, heterocycloalkyl, heterocycloalkenyl, heterocycloalkynyl, aryl, heteroaryl, alkylene, alkenylene, alkynylene, arylene, heteroarylene, cycloalkylene, heterocycloalkylene; —(CH2)m-, wherein one or more CH2 are replaced by one or more groups selected from NH and C═O, m is an integer of 0-20; —(CH2)n-heteroaryl, wherein one or more CH2 are replaced by one or more groups selected from NH, C═O and —O—, n is an integer of 0-20; —(CH2)o-heterocycloalkyl-(CH2)o-CH3, wherein one or more CH2 are replaced by one or more groups selected from NH and C═O, o is an integer of 0-20; —(CH2)q-cycloalkyl-(CH2)q-CH3, wherein one or more CH2 are replaced by one or more groups selected from NH and C═O, p is an integer of 0-20; —(CH2)r-cycloalkyl-(CH2)r-heteroaryl, wherein one or more CH2 are replaced by one or more groups selected from NH and C═O, r is an integer of 0-20; peptide; oligosaccharide, polyethylene glycol (PEG), and the combinations thereof, and said alkyl, alkenyl, alkynyl, cycloalkyl, cycloalkenyl, cycloalkynyl, heterocycloalkyl, heterocycloalkynyl, aryl, heteroaryl, —CH3, heterocycloalkenyl, alkylene, alkenylene, alkynylene, arylene, heteroarylene, cycloalkylene, heterocycloalkylene are optionally substituted by at least one substituents; D is independently active reagent, wherein said active reagent includes an anti-cancer reagent such as Mertansine and MMAE; an anti-inflammation reagent; Fluorescein such as FTIC; a peptide; a protein; a nucleotide; an oligonucleotide; a chemotherapy drug; a natural product; an immune modulator; a tubulin-binder; a DNA-alkylating agent; an HSP90 inhibitor; a DNA topoisomerase inhibitor; an anti-epigenetic agent; an HDAC inhibitor; an anti-metabolism agent; a proteasome inhibitor; a peptidomimetic; an siRNA; an antisense DNA; epothilone A, epothilone B, or paclitaxel; p is a integer refers to the number of active reagent attached to cell binding reagent, wherein p is an integer of 0-15 (including 0, 1, 2, 3, 4, 5, 6, 7, 8, 9, 10, 11, 12, 13, 14 or 15, and the range between said integers).

In some embodiments, said substituents includes but not limits to halogen, halo(C₁₋₆)alkyl, C₁₋₆ alkyl, (C₃₋₇)cycloalkyl, C₂₋₆ alkenyl, C₁₋₆ alkoxy, (C₁₋₆)alkoxy(C₁₋₆)alkyl, amino-(C₁₋₆)alkyl, C₁_6 alkylamino, di(C₁₋₆)alkylamino, (C₁₋₆)alkoxy(C₁₋₆)alkylamino, formyl, acetyl, C₂₋₆ alkylcarbonyl, (C₂₋₆)alkyl-carbonyloxy(C₁₋₆)alkyl, C₂₋₆ alkoxycarbonyl, C₂₋₆ alkoxycarbonyl(C₁₋₆)alkyl, (C₁₋₆)alkoxyamino, aminocarbonyl or amido.

In some embodiments, wherein L includes —(CH2)m-, wherein one or more CH2 are replaced by one or more groups selected from NH and C═O, m is an integer of 0-15 (including 0, 1, 2, 3, 4, 5, 6, 7, 8, 9, 10, 11, 12, 13, 14 or 15, and the range between said integers); —(CH2)n-heteroaryl, wherein one or more CH2 are replaced by one or more groups selected from NH, C═O and —O—, n is an integer of 0-20 (including 0, 1, 2, 3, 4, 5, 6, 7, 8, 9, 10, 11, 12, 13, 14, 15, 16, 17, 18, 19 or 20, and the range between said integers), and the heteroaryl group has 5 to 10 ring members; —(CH2)o-C₃₋₇heterocycloalkyl-(CH2)o-CH3, wherein one or more CH2 are replaced by one or more groups selected from NH and C═O, o is an integer of 0-15; —(CH2)q-C₃₋₇cycloalkyl-(CH2)q-CH3, wherein one or more CH2 are replaced by one or more groups selected from NH and C═O, q is an integer of 0-15 (including 0, 1, 2, 3, 4, 5, 6, 7, 8, 9, 10, 11, 12, 13, 14 or 15, and the range between said integers); —(CH2)r-C₃₋₇cycloalkyl-(CH2)r-heteroaryl, wherein one or more CH2 are replaced by one or more groups selected from NH and C═O, r is an integer of 0-15 (including 0, 1, 2, 3, 4, 5, 6, 7, 8, 9, 10, 11, 12, 13, 14 or 15, and the range between said integers), and the heteroaryl group has 5 to 10 ring members, and said cycloalkyl, heterocycloalkyl, —CH3 and heteroaryl are optionally substituted by oxo and halogen.

In some embodiments, wherein L includes —(CH2)m-, wherein one or more CH2 are replaced by one or more groups selected from NH and C═O, m is an integer of 0-10 (including 0, 1, 2, 3, 4, 5, 6, 7, 8, 9 or 10, and the range between said integers); —(CH2)n-heteroaryl, wherein one or more CH2 are replaced by one or more groups selected from NH, C═O and —O—, n is an integer of 0-20, and the heteroaryl has

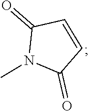

—(CH2)o-piperazinyl-(CH2)o-CH3, wherein one or more CH2 are replaced by one or more groups selected from NH and C═O, o is an integer of 0-10 (including 0, 1, 2, 3, 4, 5, 6, 7, 8, 9 or 10, and the range between said integers); —(CH2)q-cyclohexyl-(CH2)q-CH3, wherein one or more CH2 are replaced by one or more groups selected from NH and C═O, q is an integer of 0-10 (including 0, 1, 2, 3, 4, 5, 6, 7, 8, 9 or 10, and the range between said integers); —(CH2)r-cyclohexyl-(CH2)r-heteroaryl, wherein one or more CH2 are replaced by one or more groups selected from NH and C═O, r is an integer of 0-10 (including 0, 1, 2, 3, 4, 5, 6, 7, 8, 9 or 10, and the range between said integers), and the heteroaryl has

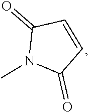

and said —CH3- is optionally substituted by halogen.

In some embodiments, wherein L includes —(CH2)m-, wherein no more than CH2 are replaced by one or more groups selected from NH and C═O, m is an integer of 0-10 (including 0, 1, 2, 3, 4, 5, 6, 7, 8, 9 or 10, and the range between said integers); —(CH2)n-heteroaryl, wherein no more than eight CH2 are replaced by one or more groups selected from NH, C═O and —O—, n is an integer of 0-18 (including 0, 1, 2, 3, 4, 5, 6, 7, 8, 9, 10, 11, 12, 13, 14, 15, 16, 17 or 18, and the range between said integers), and the heteroaryl has

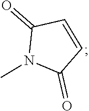

—(CH2)o-piperazinyl-(CH2)o-CH3, wherein one CH2 is replaced by one group selected from NH and C═O, o is an integer of 0-5 (including 0, 1, 2, 3, 4 or 5, and the range between said integers); —(CH2)q-cyclohexyl-(CH2)q-CH3, wherein no more than six CH2 are replaced by one or more groups selected from NH and C═O, q is an integer of 0-8 (including 0, 1, 2, 3, 4, 5, 6, 7 or 8, and the range between said integers); —(CH2)r-cyclohexyl-(CH2)r-heteroaryl, wherein no more than five CH2 are replaced by one or more groups selected from NH and C═O, r is an integer of 0-6 (including 0, 1, 2, 3, 4, 5 or 6, and the range between said integers), and the heteroaryl has

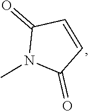

and said —CH3- is optionally substituted by Cl.

In some embodiments, D is independently active reagent, wherein said active reagent is selected from a group consisting of PBD dimer, Camptothecin Derivatives, Doxorubicin Derivatives, Calicheamicin, Duocarmycin Derivatives, Amanitin, Tubulysin Derivatives, Diphtheria toxin, Azonafide, Budesonide, Dasatinib, Desacetylvinblastine hydrazide, Dexamethasone, Hemiasterlin Analogue, Eribulin, FK506, Na, K-ATP inhibitor, Nigrin B, Phthalocyanine dye, PM050489, Rifalogue, Steroidal glycoside, Thienoindole, Yttrium-90.

In some embodiments, wherein the OPA-L-D is selected from the group consisting of:

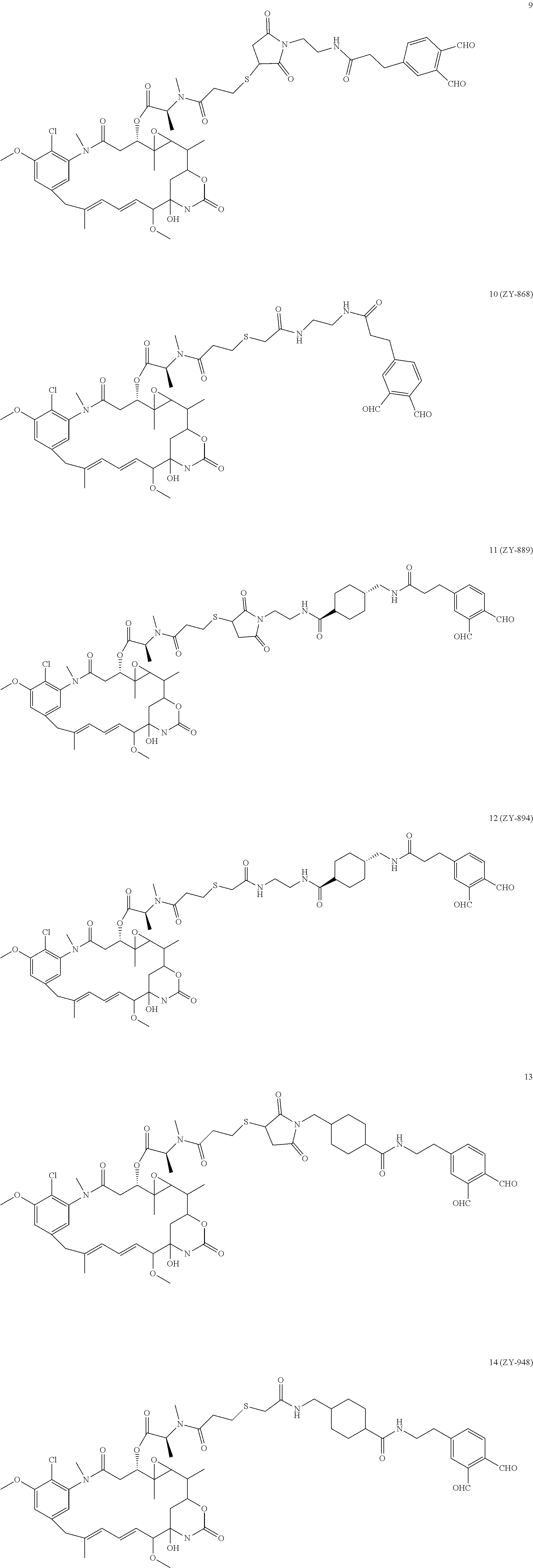

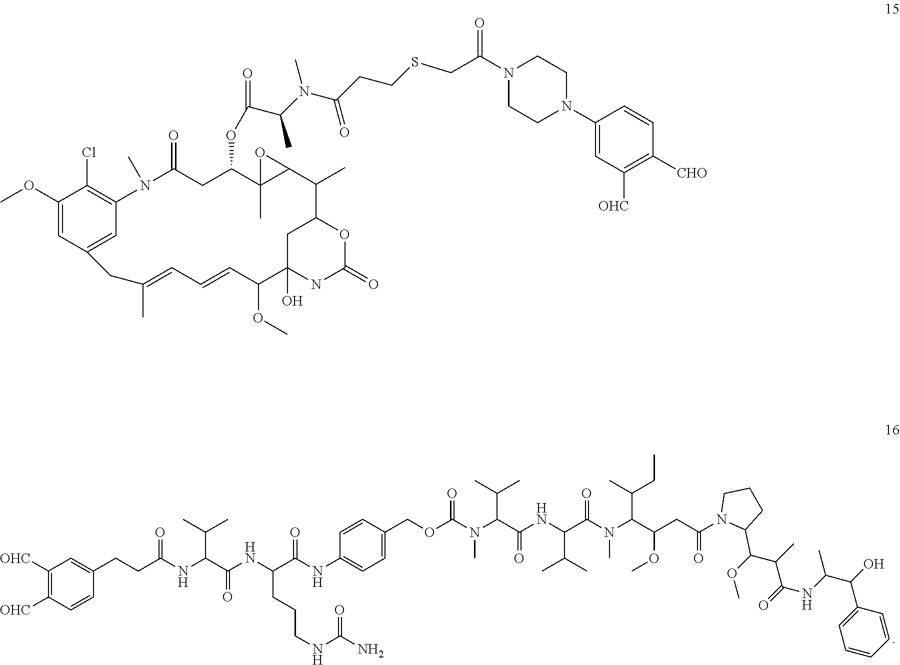 [SMILES: COc1cc2cc(c1Cl)N(C)C(=O)C[C@H](OC(=O)[C@H](C)N(C)C(=O)CCSCC(=O)N1CCN(c3ccc(C=O)c(C=O)c3)CC1)C1(C)OC1C(C)C1CC(O)(NC(=O)O1)C(OC)/C=C/C=C(\C)C2 ; CCC(C)C(C(CC(=O)N1CCCC1C(OC)C(C)C(=O)NC(C)C(O)c1ccccc1)OC)N(C)C(=O)C(NC(=O)C(C(C)C)N(C)C(=O)OCc1ccc(NC(=O)C(CCCNC(N)=O)NC(=O)C(NC(=O)CCc2ccc(C=O)c(C=O)c2)C(C)C)cc1)C(C)C]

In some embodiments, Ab is a cell binding reagent, wherein said cell binding reagent includes IgGs selecting from the group consisting of: IgG 1 (such as, Trastuzumab or Cetuximab), IgG 2, IgG 3, IgG 4 (such as, Inotuzumab) and said Heavy-chain only antibody includes but not limits to HcAb-1 or HcAb-2.

In some embodiments, the compound of formula (III) includes but not limits to Trastuzumab-ZY-889, Trastuzumab-ZY-948, Trastuzumab-ZY-868, Trastuzumab-ZY-894, Erbitux-ZY-889, Inotuzumab-Zy-889, HcAb-1-ZY-894 and HcAb-2-ZY-894.

In some embodiments, the compound of formula (III) is selected from the group consisting of: Trastuzumab-ZY-889, Trastuzumab-ZY-948, HcAb-1-ZY-894, HcAb-2-ZY-894 and HcAb-2-ZY-894.

In the fourth aspect of the present application relates to a use of a compound of formula OPA-L (I) as defined above for the preparation of a conjugate of the following formula (III): Ab-(OPA-L-D)p (III), wherein Ab, OPA, L, D and p are defined as above.

In some embodiments, OPA is

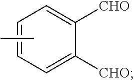

L includes alkyl, alkenyl, alkynyl, cycloalkyl, cycloalkenyl, cycloalkynyl, heterocycloalkyl, heterocycloalkenyl, heterocycloalkynyl, aryl, heteroaryl, alkylene, alkenylene, alkynylene, arylene, heteroarylene, cycloalkylene, heterocycloalkylene; —(CH2)m-, wherein one or more CH2 are replaced by one or more groups selected from NH and C═O, m is an integer of 0-20 (including 0, 1, 2, 3, 4, 5, 6, 7, 8, 9, 10, 11, 12, 13, 14, 15, 16, 17, 18, 19 or 20, and the range between said integers); —(CH2)n-heteroaryl, wherein one or more CH2 are replaced by one or more groups selected from —NH, —C═O and —O—, n is an integer of 0-20 (including 0, 1, 2, 3, 4, 5, 6, 7, 8, 9, 10, 11, 12, 13, 14, 15, 16, 17, 18, 19 or 20, and the range between said integers); —(CH2)o-heterocycloalkyl-(CH2)o-CH3, wherein one or more CH2 are replaced by one or more groups selected from NH and C═O, o is an integer of 0-20; —(CH2)q-cycloalkyl-(CH2)q-CH3, wherein one or more CH2 are replaced by one or more groups selected from NH and C═O, q is an integer of 0-20 (including 0, 1, 2, 3, 4, 5, 6, 7, 8, 9, 10, 11, 12, 13, 14, 15, 16, 17, 18, 19 or 20, and the range between said integers); —(CH2)r-cycloalkyl-(CH2)r-heteroaryl, wherein one or more CH2 are replaced by one or more groups selected from NH and C═O, r is an integer of 0-20 (including 0, 1, 2, 3, 4, 5, 6, 7, 8, 9, 10, 11, 12, 13, 14, 15, 16, 17, 18, 19 or 20, and the range between said integers); peptide like di-peptides, tri-peptides, tetra-peptide, penta-peptide; oligosaccharide, polyethylene glycol (PEG), and the combinations thereof, and said alkyl, alkenyl, alkynyl, cycloalkyl, cycloalkenyl, cycloalkynyl, heterocycloalkyl, heterocycloalkynyl, aryl, heteroaryl, —CH3, heterocycloalkenyl, alkylene, alkenylene, alkynylene, arylene, heteroarylene, cycloalkylene, heterocycloalkylene are optionally substituted by at least one substituents; D is independently active reagent, wherein said active reagent includes an anti-cancer reagent such as Mertansine and MMAE; an anti-inflammation reagent; Fluorescein such as FTIC; a peptide; a protein; a nucleotide; an oligonucleotide; a chemotherapy drug; a natural product; an immune modulator; a tubulin-binder; a DNA-alkylating agent; an HSP90 inhibitor; a DNA topoisomerase inhibitor; an anti-epigenetic agent; an HDAC inhibitor; an anti-metabolism agent; a proteasome inhibitor; a peptidomimetic; an siRNA; an antisense DNA; epothilone A, epothilone B, or paclitaxel.

In some embodiments, said substituents includes but not limits to halogen, halo(C₁₋₆)alkyl, C₁₋₆ alkyl, (C₃₋₇)cycloalkyl, C₂₋₆ alkenyl, C₁₋₆ alkoxy, (C₁₋₆)alkoxy(C₁₋₆)alkyl, amino-(C₁₋₆)alkyl, C₁_6 alkylamino, di(C₁₋₆)alkylamino, (C₁₋₆)alkoxy(C₁₋₆)alkylamino, formyl, acetyl, C₂₋₆ alkylcarbonyl, (C₂₋₆)alkyl-carbonyloxy(C₁₋₆)alkyl, C₂₋₆ alkoxycarbonyl, C₂₋₆ alkoxycarbonyl(C₁₋₆)alkyl, (C₁₋₆)alkoxyamino, aminocarbonyl or amido.

In some embodiments, L includes —(CH2)m-, wherein one or more CH2 are replaced by one or more groups selected from NH and C═O, m is an integer of 0-15 (including 0, 1, 2, 3, 4, 5, 6, 7, 8, 9, 10, 11, 12, 13, 14 or 15, and the range between said integers); —(CH2)n-heteroaryl, wherein one or more CH2 are replaced by one or more groups selected from NH, C═O and —O—, n is an integer of 0-20 (including 0, 1, 2, 3, 4, 5, 6, 7, 8, 9, 10, 11, 12, 13, 14, 15, 16, 17, 18, 19 or 20, and the range between said integers), and the heteroaryl group has 5 to 10 ring members; —(CH2)o-C₃₋₇heterocycloalkyl-(CH2)o-CH3, wherein one or more CH2 are replaced by one or more groups selected from NH and C═O, o is an integer of 0-15 (including 0, 1, 2, 3, 4, 5, 6, 7, 8, 9, 10, 11, 12, 13, 14 or 15, and the range between said integers); —(CH2)q-C₃₋₇cycloalkyl-(CH2)q-CH3, wherein one or more CH2 are replaced by one or more groups selected from NH and C═O, q is an integer of 0-15 (including 0, 1, 2, 3, 4, 5, 6, 7, 8, 9, 10, 11, 12, 13, 14 or 15, and the range between said integers); —(CH2)r-C₃₋₇cycloalkyl-(CH2)r-heteroaryl, wherein one or more CH2 are replaced by one or more groups selected from NH and C═O, r is an integer of 0-15 (including 0, 1, 2, 3, 4, 5, 6, 7, 8, 9, 10, 11, 12, 13, 14 or 15, and the range between said integers), and the heteroaryl group has 5 to 10 ring members, and said cycloalkyl, heterocycloalkyl, —CH3 and heteroaryl are optionally substituted by oxo and halogen.

In some embodiments, wherein L includes —(CH2)m-, wherein one or more CH2 are replaced by one or more groups selected from NH and C═O, m is an integer of 0-10 (including 0, 1, 2, 3, 4, 5, 6, 7, 8, 9 or 10, and the range between said integers); —(CH2)n-heteroaryl, wherein one or more CH2 are replaced by one or more groups selected from NH, C═O and —O—, n is an integer of 0-20 (including 0, 1, 2, 3, 4, 5, 6, 7, 8, 9, 10, 11, 12, 13, 14, 15, 16, 17, 18, 19 or 20, and the range between said integers), and the heteroaryl has

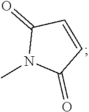

—(CH2)o-piperazinyl-(CH2)o-CH3, wherein one or more CH2 are replaced by one or more groups selected from NH and C═O, o is an integer of 0-10 (including 0, 1, 2, 3, 4, 5, 6, 7, 8, 9 or 10, and the range between said integers); —(CH2)q-cyclohexyl-(CH2)q-CH3, wherein one or more CH2 are replaced by one or more groups selected from NH and C═O, q is an integer of 0-10; —(CH2)r-cyclohexyl-(CH2)r-heteroaryl, wherein one or more CH2 are replaced by one or more groups selected from NH and C═O, r is an integer of 0-10 (including 0, 1, 2, 3, 4, 5, 6, 7, 8, 9 or 10, and the range between said integers), and the heteroaryl has

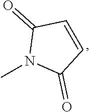

and said —CH3- is optionally substituted by halogen.

In some embodiments, wherein L includes —(CH2)m-, wherein no more than CH2 are replaced by one or more groups selected from NH and C═O, m is an integer of 0-10 (including 0, 1, 2, 3, 4, 5, 6, 7, 8, 9 or 10, and the range between said integers); —(CH2)n-heteroaryl, wherein no more than eight CH2 are replaced by one or more groups selected from NH, C═O and —O—, n is an integer of 0-18 (including 0, 1, 2, 3, 4, 5, 6, 7, 8, 9, 10, 11, 12, 13, 14, 15, 16, 17 or 18, and the range between said integers), and the heteroaryl has

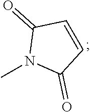

—(CH2)o-piperazinyl-(CH2)o-CH3, wherein one CH2 is replaced by one group selected from NH and C═O, o is an integer of 0-5 (including 0, 1, 2, 3, 4 or 5, and the range between said integers); —(CH2)q-cyclohexyl-(CH2)q-CH3, wherein no more than six CH2 are replaced by one or more groups selected from NH and C═O, q is an integer of 0-8 (including 0, 1, 2, 3, 4, 5, 6, 7 or 8, and the range between said integers); —(CH2)r-cyclohexyl-(CH2)r-heteroaryl, wherein no more than five CH2 are replaced by one or more groups selected from NH and C═O, r is an integer of 0-6 (including 0, 1, 2, 3, 4, 5 or 6, and the range between said integers), and the heteroaryl has

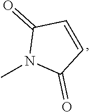

and said —CH3- is optionally substituted by Cl.

In some embodiments, wherein the OPA-L is selected from the group consisting of:

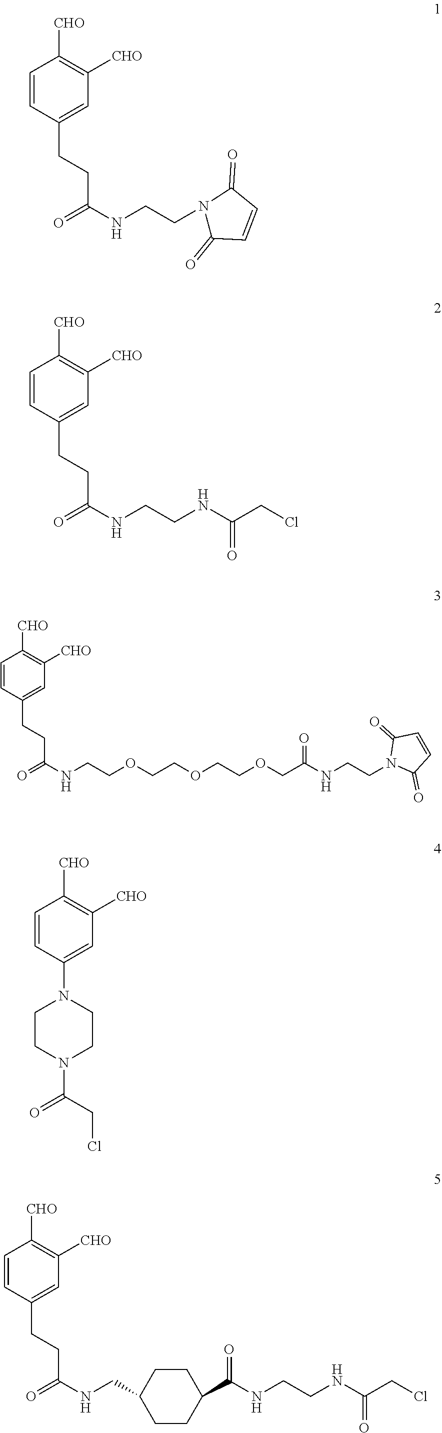 [SMILES: O=Cc1ccc(CCC(=O)NCCN2C(=O)C=CC2=O)cc1C=O ; O=Cc1ccc(CCC(=O)NCCNC(=O)CCl)cc1C=O ; O=Cc1ccc(CCC(=O)NCCOCCOCCOCC(=O)NCCN2C(=O)C=CC2=O)cc1C=O ; O=Cc1ccc(N2CCN(C(=O)CCl)CC2)cc1C=O ; O=Cc1ccc(CCC(=O)NC[C@H]2CC[C@H](C(=O)NCCNC(=O)CCl)CC2)cc1C=O]

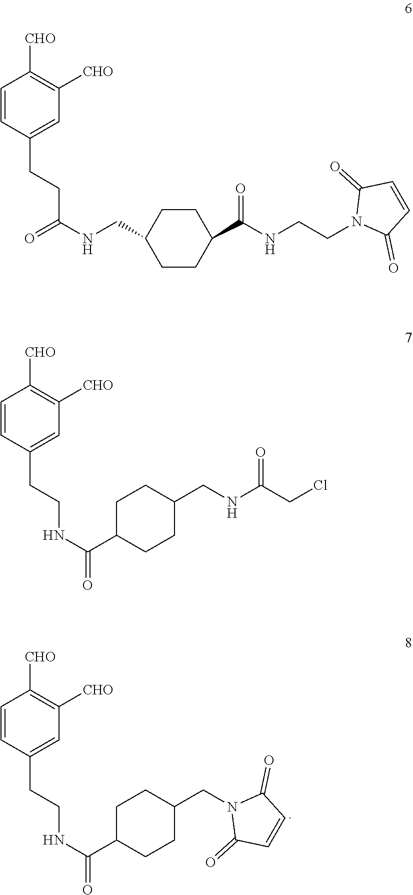 [SMILES: O=Cc1ccc(CCC(=O)NC[C@H]2CC[C@H](C(=O)NCCN3C(=O)C=CC3=O)CC2)cc1C=O ; O=Cc1ccc(CCNC(=O)C2CCC(CNC(=O)CCl)CC2)cc1C=O ; O=Cc1ccc(CCNC(=O)C2CCC(CN3C(=O)C=CC3=O)CC2)cc1C=O]

In some embodiments, D is independently active reagent, wherein said active reagent is selected from a group consisting of PBD dimer, Camptothecin Derivatives, Doxorubicin Derivatives, Calicheamicin, Duocarmycin Derivatives, Amanitin, Tubulysin Derivatives, Diphtheria toxin, Azonafide, Budesonide, Dasatinib, Desacetylvinblastine hydrazide, Dexamethasone, Hemiasterlin Analogue, Eribulin, FK506, Na, K-ATP inhibitor, Nigrin B, Phthalocyanine dye, PM050489, Rifalogue, Steroidal glycoside, Thienoindole, Yttrium-90.

In some embodiments, Ab is a cell binding reagent, wherein said cell binding reagent includes IgGs selecting from the group consisting of: IgG 1 (such as, Trastuzumab or Cetuximab), IgG 2, IgG 3, IgG 4 (such as, Inotuzumab) and said Heavy-chain only antibody includes but not limits to HcAb-1 or HcAb-2.

In some embodiments, the compound of formula (III) includes but not limits to Trastuzumab-ZY-889, Trastuzumab-ZY-948, Trastuzumab-ZY-868, Trastuzumab-ZY-894, Erbitux-ZY-889, Inotuzumab-Zy-889, HcAb-1-ZY-894 and HcAb-2-ZY-894.

In some embodiments, the compound of formula (III) is selected from the group consisting of: Trastuzumab-ZY-889, Trastuzumab-ZY-948, HcAb-1-ZY-894, HcAb-2-ZY-894 and HcAb-2-ZY-894.

In the fifth aspect of the present application relates to a use of a compound of formula OPA-L-D (II) as defined above for the preparation of a conjugate of the following formula (III): Ab-(OPA-L-D)p (Ill), wherein Ab, OPA, L, D and p are defined as above.

In some embodiments, OPA is

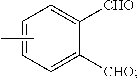

L includes alkyl, alkenyl, alkynyl, cycloalkyl, cycloalkenyl, cycloalkynyl, heterocycloalkyl, heterocycloalkenyl, heterocycloalkynyl, aryl, heteroaryl, alkylene, alkenylene, alkynylene, arylene, heteroarylene, cycloalkylene, heterocycloalkylene; —(CH2)m-, wherein one or more CH2 are replaced by one or more groups selected from NH and C═O, m is an integer of 0-20 (including 0, 1, 2, 3, 4, 5, 6, 7, 8, 9, 10, 11, 12, 13, 14, 15, 16, 17, 18, 19 or 20, and the range between said integers); —(CH2)n-heteroaryl, wherein one or more CH2 are replaced by one or more groups selected from NH, C═O and —O—, n is an integer of 0-20 (including 0, 1, 2, 3, 4, 5, 6, 7, 8, 9, 10, 11, 12, 13, 14, 15, 16, 17, 18, 19 or 20, and the range between said integers); —(CH2)o-heterocycloalkyl-(CH2)o-CH3, wherein one or more CH2 are replaced by one or more groups selected from NH and C═O, o is an integer of 0-20 (including 0, 1, 2, 3, 4, 5, 6, 7, 8, 9, 10, 11, 12, 13, 14, 15, 16, 17, 18, 19 or 20, and the range between said integers); —(CH2)q-cycloalkyl-(CH2)q-CH3, wherein one or more CH2 are replaced by one or more groups selected from NH and C═O, q is an integer of 0-20 (including 0, 1, 2, 3, 4, 5, 6, 7, 8, 9, 10, 11, 12, 13, 14, 15, 16, 17, 18, 19 or 20, and the range between said integers); —(CH2)r-cycloalkyl-(CH2)r-heteroaryl, wherein one or more CH2 are replaced by one or more groups selected from NH and C═O, r is an integer of 0-20 (including 0, 1, 2, 3, 4, 5, 6, 7, 8, 9, 10, 11, 12, 13, 14, 15, 16, 17, 18, 19 or 20, and the range between said integers); peptide; oligosaccharide, polyethylene glycol (PEG), and the combinations thereof, and said alkyl, alkenyl, alkynyl, cycloalkyl, cycloalkenyl, cycloalkynyl, heterocycloalkyl, heterocycloalkynyl, aryl, heteroaryl, —CH3, heterocycloalkenyl, alkylene, alkenylene, alkynylene, arylene, heteroarylene, cycloalkylene, heterocycloalkylene are optionally substituted by at least one substituents; D is independently active reagent, wherein said active reagent includes an anti-cancer reagent such as Mertansine and MMAE; an anti-inflammation reagent; Fluorescein such as FTIC; a peptide; a protein; a nucleotide; an oligonucleotide; a chemotherapy drug; a natural product; an immune modulator; a tubulin-binder; a DNA-alkylating agent; an HSP90 inhibitor; a DNA topoisomerase inhibitor; an anti-epigenetic agent; an HDAC inhibitor; an anti-metabolism agent; a proteasome inhibitor; a peptidomimetic; an siRNA; an antisense DNA; epothilone A, epothilone B, or paclitaxel.

In some embodiments, said substituents includes but not limits to halogen, halo(C₁₋₆)alkyl, C₁₋₆ alkyl, (C₃₋₇)cycloalkyl, C₂₋₆ alkenyl, C₁₋₆ alkoxy, (C₁₋₆)alkoxy(C₁₋₆)alkyl, amino-(C₁₋₆)alkyl, C₁_6 alkylamino, di(C₁₋₆)alkylamino, (C₁₋₆)alkoxy(C₁₋₆)alkylamino, formyl, acetyl, C₂₋₆ alkylcarbonyl, (C₂₋₆)alkyl-carbonyloxy(C₁₋₆)alkyl, C₂₋₆ alkoxycarbonyl, C₂₋₆ alkoxycarbonyl(C₁₋₆)alkyl, (C₁₋₆)alkoxyamino, aminocarbonyl or amido.

In some embodiments, wherein L includes —(CH2)m-, wherein one or more CH2 are replaced by one or more groups selected from NH and C═O, m is an integer of 0-15 (including 0, 1, 2, 3, 4, 5, 6, 7, 8, 9, 10, 11, 12, 13, 14 or 15, and the range between said integers); —(CH2)n-heteroaryl, wherein one or more CH2 are replaced by one or more groups selected from NH, C═O and —O—, n is an integer of 0-20 (including 0, 1, 2, 3, 4, 5, 6, 7, 8, 9, 10, 11, 12, 13, 14, 15, 16, 17, 18, 19 or 20, and the range between said integers), and the heteroaryl group has 5 to 10 ring members; —(CH2)o-C₃₋₇heterocycloalkyl-(CH2)o-CH3, wherein one or more CH2 are replaced by one or more groups selected from NH and C═O, o is an integer of 0-15; —(CH2)q-C₃₋₇cycloalkyl-(CH2)q-CH3, wherein one or more CH2 are replaced by one or more groups selected from NH and C═O, q is an integer of 0-15 (including 0, 1, 2, 3, 4, 5, 6, 7, 8, 9, 10, 11, 12, 13, 14 or 15, and the range between said integers); —(CH2)r-C₃₋₇cycloalkyl-(CH2)r-heteroaryl, wherein one or more CH2 are replaced by one or more groups selected from NH and C═O, r is an integer of 0-15 (including 0, 1, 2, 3, 4, 5, 6, 7, 8, 9, 10, 11, 12, 13, 14 or 15, and the range between said integers), and the heteroaryl group has 5 to 10 ring members, and said cycloalkyl, heterocycloalkyl, —CH3 and heteroaryl are optionally substituted by oxo and halogen.

In some embodiments, wherein L includes —(CH2)m-, wherein one or more CH2 are replaced by one or more groups selected from NH and C═O, m is an integer of 0-10 (including 0, 1, 2, 3, 4, 5, 6, 7, 8, 9 or 10, and the range between said integers); —(CH2)n-heteroaryl, wherein one or more CH2 are replaced by one or more groups selected from NH, C═O and —O—, n is an integer of 0-20, and the heteroaryl has

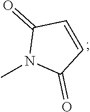

—(CH2)o-piperazinyl-(CH2)o-CH3, wherein one or more CH2 are replaced by one or more groups selected from NH and C═O, o is an integer of 0-10 (including 0, 1, 2, 3, 4, 5, 6, 7, 8, 9 or 10, and the range between said integers); —(CH2)q-cyclohexyl-(CH2)q-CH3, wherein one or more CH2 are replaced by one or more groups selected from NH and C═O, q is an integer of 0-10 (including 0, 1, 2, 3, 4, 5, 6, 7, 8, 9 or 10, and the range between said integers); —(CH2)r-cyclohexyl-(CH2)r-heteroaryl, wherein one or more CH2 are replaced by one or more groups selected from NH and C═O, r is an integer of 0-10 (including 0, 1, 2, 3, 4, 5, 6, 7, 8, 9 or 10, and the range between said integers), and the heteroaryl has

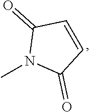

and said —CH3- is optionally substituted by halogen.

In some embodiments, wherein L includes —(CH2)m-, wherein no more than CH2 are replaced by one or more groups selected from NH and C═O, m is an integer of 0-10 (including 0, 1, 2, 3, 4, 5, 6, 7, 8, 9 or 10, and the range between said integers); —(CH2)n-heteroaryl, wherein no more than eight CH2 are replaced by one or more groups selected from NH, C═O and —O—, n is an integer of 0-18 (including 0, 1, 2, 3, 4, 5, 6, 7, 8, 9, 10, 11, 12, 13, 14, 15, 16, 17 or 18, and the range between said integers), and the heteroaryl has

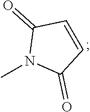

—(CH2)o-piperazinyl-(CH2)o-CH3, wherein one CH2 is replaced by one group selected from NH and C═O, o is an integer of 0-5 (including 0, 1, 2, 3, 4 or 5, and the range between said integers); —(CH2)q-cyclohexyl-(CH2)q-CH3, wherein no more than six CH2 are replaced by one or more groups selected from NH and C═O, q is an integer of 0-8 (including 0, 1, 2, 3, 4, 5, 6, 7 or 8, and the range between said integers); —(CH2)r-cyclohexyl-(CH2)r-heteroaryl, wherein no more than five CH2 are replaced by one or more groups selected from NH and C═O, r is an integer of 0-6 (including 0, 1, 2, 3, 4, 5 or 6, and the range between said integers), and the heteroaryl has

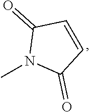

and said —CH3- is optionally substituted by Cl.

In some embodiments, wherein the OPA-L-D is selected from the group consisting of:

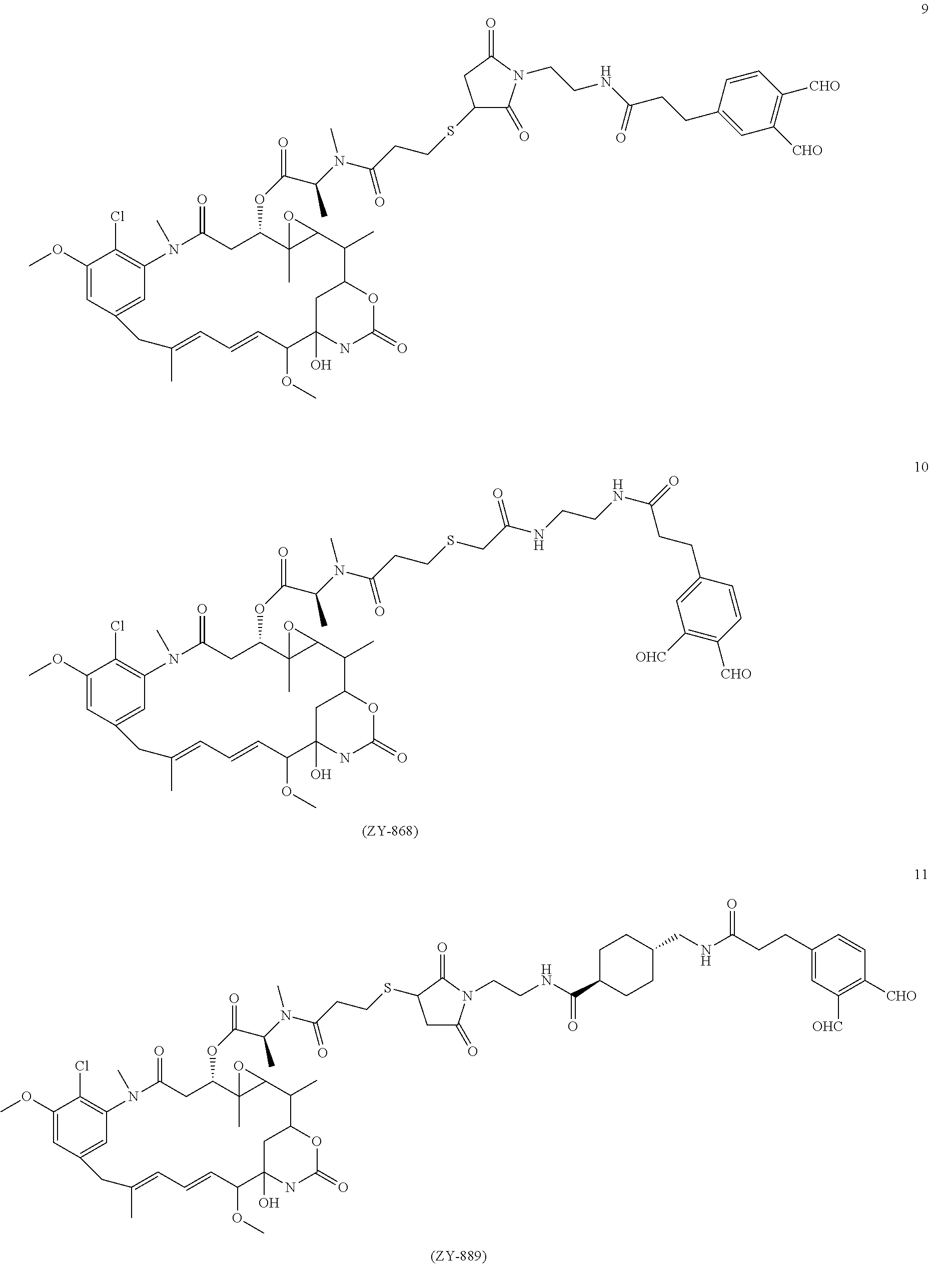 [SMILES: COc1cc2cc(c1Cl)N(C)C(=O)C[C@H](OC(=O)[C@H](C)N(C)C(=O)CCSC1CC(=O)N(CCNC(=O)CCc3ccc(C=O)c(C=O)c3)C1=O)C1(C)OC1C(C)C1CC(O)(NC(=O)O1)C(OC)/C=C/C=C(\C)C2 ; COc1cc2cc(c1Cl)N(C)C(=O)C[C@H](OC(=O)[C@H](C)N(C)C(=O)CCSCC(=O)NCCNC(=O)CCc1ccc(C=O)c(C=O)c1)C1(C)OC1C(C)C1CC(O)(NC(=O)O1)C(OC)/C=C/C=C(\C)C2 ; COc1cc2cc(c1Cl)N(C)C(=O)C[C@H](OC(=O)[C@H](C)N(C)C(=O)CCSC1CC(=O)N(CCNC(=O)[C@H]3CC[C@H](CNC(=O)CCc4ccc(C=O)c(C=O)c4)CC3)C1=O)C1(C)OC1C(C)C1CC(O)(NC(=O)O1)C(OC)/C=C/C=C(\C)C2]

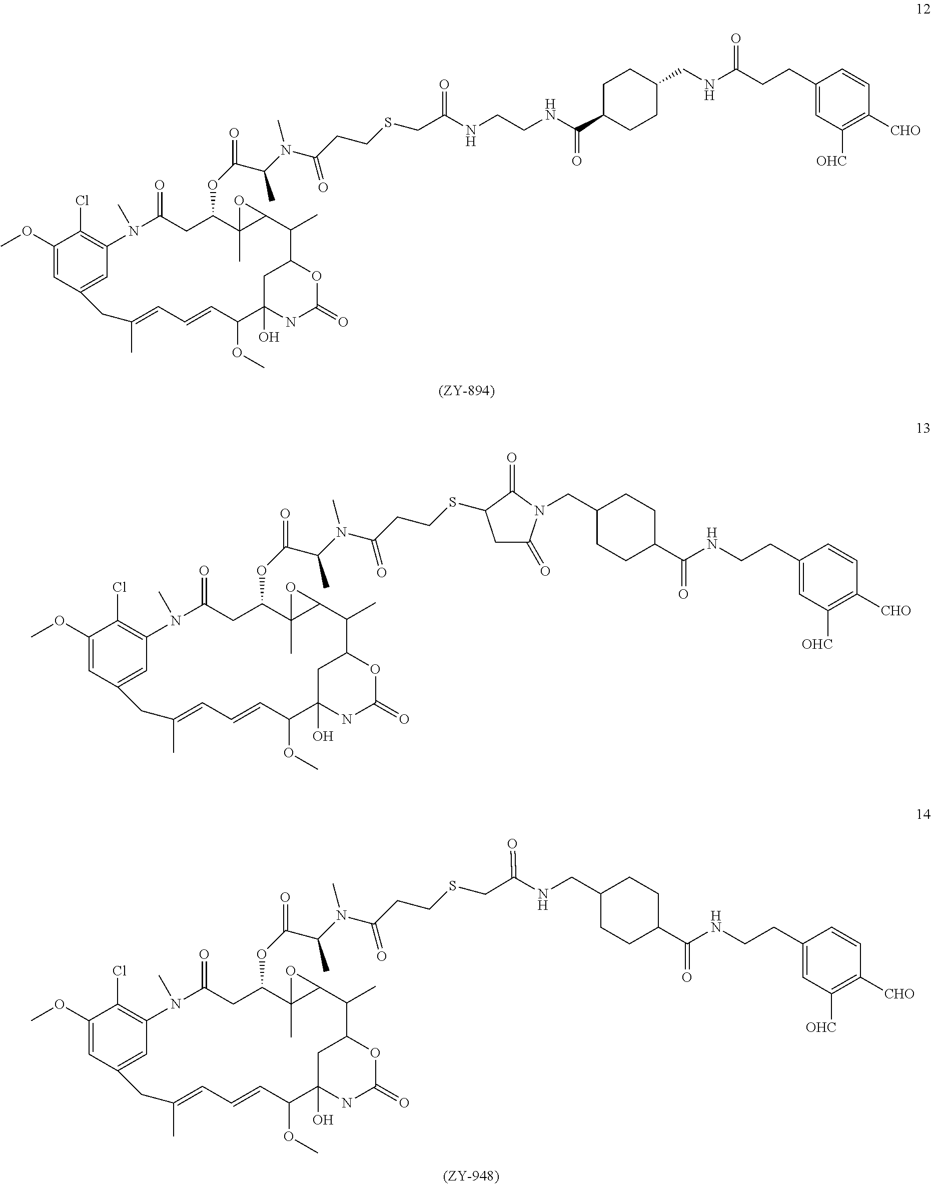 [SMILES: COc1cc2cc(c1Cl)N(C)C(=O)C[C@H](OC(=O)[C@H](C)N(C)C(=O)CCSCC(=O)NCCNC(=O)[C@H]1CC[C@H](CNC(=O)CCc3ccc(C=O)c(C=O)c3)CC1)C1(C)OC1C(C)C1CC(O)(NC(=O)O1)C(OC)/C=C/C=C(\C)C2 ; COc1cc2cc(c1Cl)N(C)C(=O)C[C@H](OC(=O)[C@H](C)N(C)C(=O)CCSC1CC(=O)N(CC3CCC(C(=O)NCCc4ccc(C=O)c(C=O)c4)CC3)C1=O)C1(C)OC1C(C)C1CC(O)(NC(=O)O1)C(OC)/C=C/C=C(\C)C2 ; COc1cc2cc(c1Cl)N(C)C(=O)C[C@H](OC(=O)[C@H](C)N(C)C(=O)CCSCC(=O)NCC1CCC(C(=O)NCCc3ccc(C=O)c(C=O)c3)CC1)C1(C)OC1C(C)C1CC(O)(NC(=O)O1)C(OC)/C=C/C=C(\C)C2]

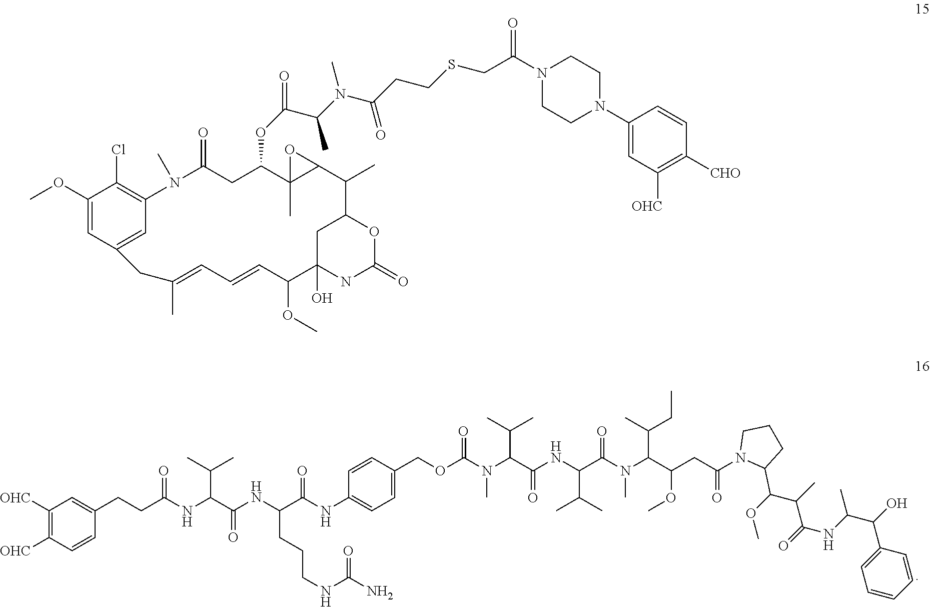 [SMILES: COc1cc2cc(c1Cl)N(C)C(=O)C[C@H](OC(=O)[C@H](C)N(C)C(=O)CCSCC(=O)N1CCN(c3ccc(C=O)c(C=O)c3)CC1)C1(C)OC1C(C)C1CC(O)(NC(=O)O1)C(OC)/C=C/C=C(\C)C2 ; CCC(C)C(C(CC(=O)N1CCCC1C(OC)C(C)C(=O)NC(C)C(O)c1ccccc1)OC)N(C)C(=O)C(NC(=O)C(C(C)C)N(C)C(=O)OCc1ccc(NC(=O)C(CCCNC(N)=O)NC(=O)C(NC(=O)CCc2ccc(C=O)c(C=O)c2)C(C)C)cc1)C(C)C]

In some embodiments, D is independently active reagent, wherein said active reagent is selected from a group consisting of PBD dimer, Camptothecin Derivatives, Doxorubicin Derivatives, Calicheamicin, Duocarmycin Derivatives, Amanitin, Tubulysin Derivatives, Diphtheria toxin, Azonafide, Budesonide, Dasatinib, Desacetylvinblastine hydrazide, Dexamethasone, Hemiasterlin Analogue, Eribulin, FK506, Na, K-ATP inhibitor, Nigrin B, Phthalocyanine dye, PM050489, Rifalogue, Steroidal glycoside, Thienoindole, Yttrium-90.

In some embodiments, Ab is a cell binding reagent, wherein said cell binding reagent includes IgGs selecting from the group consisting of: IgG 1 (such as, Trastuzumab or Cetuximab), IgG 2, IgG 3, IgG 4 (such as, Inotuzumab) and said Heavy-chain only antibody includes but not limits to HcAb-1 or HcAb-2.

In some embodiments, the compound of formula (III) includes but not limits to Trastuzumab-ZY-889, Trastuzumab-ZY-948, Trastuzumab-ZY-868, Trastuzumab-ZY-894, Erbitux-ZY-889, Inotuzumab-Zy-889, HcAb-1-ZY-894 and HcAb-2-ZY-894.

In some embodiments, the compound of formula (III) is selected from the group consisting of: Trastuzumab-ZY-889, Trastuzumab-ZY-948, HcAb-1-ZY-894, HcAb-2-ZY-894 and HcAb-2-ZY-894.

In the sixth aspect of the present application relates to a process for the preparation of conjugate of the following formula (III): Ab-(OPA-L-D)p (Ill), wherein the conjugate comprises D linked to Ab through the reaction of primary amine on Ab and OPA-L, the process comprising the steps of: (a) contacting D with OPA-L to covalently attach the OPA-L to D and therefore prepare OPA-L-D, wherein D, OPA-L and OPA-L-D are defined as above; (b) conjugating Ab to OPA-L-D by reacting the OPA-L-D with Ab to prepare the conjugate of formula (III), wherein Ab and p are defined as above; and (c) purifying the conjugate of formula (III) with down-stream steps such as buffer exchange or column purification.

In some embodiments, wherein OPA is

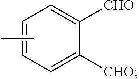

L includes alkyl, alkenyl, alkynyl, cycloalkyl, cycloalkenyl, cycloalkynyl, heterocycloalkyl, heterocycloalkenyl, heterocycloalkynyl, aryl, heteroaryl, alkylene, alkenylene, alkynylene, arylene, heteroarylene, cycloalkylene, heterocycloalkylene; —(CH2)m-, wherein one or more CH2 are replaced by one or more groups selected from NH and C═O, m is an integer of 0-20 (including 0, 1, 2, 3, 4, 5, 6, 7, 8, 9, 10, 11, 12, 13, 14, 15, 16, 17, 18, 19 or 20, and the range between said integers); —(CH2)n-heteroaryl, wherein one or more CH2 are replaced by one or more groups selected from NH, C═O and —O—, n is an integer of 0-20 (including 0, 1, 2, 3, 4, 5, 6, 7, 8, 9, 10, 11, 12, 13, 14, 15, 16, 17, 18, 19 or 20, and the range between said integers); —(CH2)o-heterocycloalkyl-(CH2)o-CH3, wherein one or more CH2 are replaced by one or more groups selected from NH and C═O, o is an integer of 0-20 (including 0, 1, 2, 3, 4, 5, 6, 7, 8, 9, 10, 11, 12, 13, 14, 15, 16, 17, 18, 19 or 20, and the range between said integers); —(CH2)q-cycloalkyl-(CH2)q-CH3, wherein one or more CH2 are replaced by one or more groups selected from NH and C═O, q is an integer of 0-20; —(CH2)r-cycloalkyl-(CH2)r-heteroaryl, wherein one or more CH2 are replaced by one or more groups selected from NH and C═O, r is an integer of 0-20 (including 0, 1, 2, 3, 4, 5, 6, 7, 8, 9, 10, 11, 12, 13, 14, 15, 16, 17, 18, 19 or 20, and the range between said integers); peptide; oligosaccharide, polyethylene glycol (PEG), and the combinations thereof, and said alkyl, alkenyl, alkynyl, cycloalkyl, cycloalkenyl, cycloalkynyl, heterocycloalkyl, heterocycloalkynyl, aryl, heteroaryl, —CH3, heterocycloalkenyl, alkylene, alkenylene, alkynylene, arylene, heteroarylene, cycloalkylene, heterocycloalkylene are optionally substituted by at least one substituents; D is independently active reagent, wherein said active reagent includes an anti-cancer reagent such as Mertansine and MMAE; an anti-inflammation reagent; Fluorescein such as FTIC; a peptide; a protein; a nucleotide; an oligonucleotide; a chemotherapy drug; a natural product; an immune modulator; a tubulin-binder; a DNA-alkylating agent; an HSP90 inhibitor; a DNA topoisomerase inhibitor; an anti-epigenetic agent; an HDAC inhibitor; an anti-metabolism agent; a proteasome inhibitor; a peptidomimetic; an siRNA; an antisense DNA; epothilone A, epothilone B, or paclitaxel.

In some embodiments, said substituents includes but not limits to halogen, halo(C₁₋₆)alkyl, C₁₋₆ alkyl, (C₃₋₇)cycloalkyl, C₂₋₆ alkenyl, C₁₋₆ alkoxy, (C₁₋₆)alkoxy(C₁₋₆)alkyl, amino-(C₁₋₆)alkyl, C₁_6 alkylamino, di(C₁₋₆)alkylamino, (C₁₋₆)alkoxy(C₁₋₆)alkylamino, formyl, acetyl, C₂₋₆ alkylcarbonyl, (C₂₋₆)alkyl-carbonyloxy(C₁₋₆)alkyl, C₂₋₆ alkoxycarbonyl, C₂₋₆ alkoxycarbonyl(C₁₋₆)alkyl, (C₁₋₆)alkoxyamino, aminocarbonyl or amido.

In some embodiments, D is independently active reagent, wherein said active reagent is selected from a group consisting of PBD dimer, Camptothecin Derivatives, Doxorubicin Derivatives, Calicheamicin, Duocarmycin Derivatives, Amanitin, Tubulysin Derivatives, Diphtheria toxin, Azonafide, Budesonide, Dasatinib, Desacetylvinblastine hydrazide, Dexamethasone, Hemiasterlin Analogue, Eribulin, FK506, Na, K-ATP inhibitor, Nigrin B, Phthalocyanine dye, PM050489, Rifalogue, Steroidal glycoside, Thienoindole, Yttrium-90.

In some embodiments, wherein L includes —(CH2)m-, wherein one or more CH2 are replaced by one or more groups selected from NH and C═O, m is an integer of 0-15 (including 0, 1, 2, 3, 4, 5, 6, 7, 8, 9, 10, 11, 12, 13, 14 or 15, and the range between said integers); —(CH2)n-heteroaryl, wherein one or more CH2 are replaced by one or more groups selected from NH, C═O and —O—, n is an integer of 0-20 (including 0, 1, 2, 3, 4, 5, 6, 7, 8, 9, 10, 11, 12, 13, 14, 15, 16, 17, 18, 19 or 20, and the range between said integers), and the heteroaryl group has 5 to 10 ring members; —(CH2)o-C₃₋₇heterocycloalkyl-(CH2)o-CH3, wherein one or more CH2 are replaced by one or more groups selected from NH and C═O, o is an integer of 0-15; —(CH2)q-C₃₋₇cycloalkyl-(CH2)q-CH3, wherein one or more CH2 are replaced by one or more groups selected from NH and C═O, q is an integer of 0-15 (including 0, 1, 2, 3, 4, 5, 6, 7, 8, 9, 10, 11, 12, 13, 14 or 15, and the range between said integers); —(CH2)r-C₃₋₇cycloalkyl-(CH2)r-heteroaryl, wherein one or more CH2 are replaced by one or more groups selected from NH and C═O, r is an integer of 0-15 (including 0, 1, 2, 3, 4, 5, 6, 7, 8, 9, 10, 11, 12, 13, 14 or 15, and the range between said integers), and the heteroaryl group has 5 to 10 ring members, and said cycloalkyl, heterocycloalkyl, —CH3 and heteroaryl are optionally substituted by oxo and halogen.

In some embodiments, wherein L includes —(CH2)m-, wherein one or more CH2 are replaced by one or more groups selected from NH and C═O, m is an integer of 0-10 (including 0, 1, 2, 3, 4, 5, 6, 7, 8, 9 or 10, and the range between said integers); —(CH2)n-heteroaryl, wherein one or more CH2 are replaced by one or more groups selected from NH, C═O and —O—, n is an integer of 0-20, and the heteroaryl has

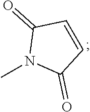

—(CH2)o-piperazinyl-(CH2)o-CH3, wherein one or more CH2 are replaced by one or more groups selected from NH and C═O, o is an integer of 0-10 (including 0, 1, 2, 3, 4, 5, 6, 7, 8, 9 or 10, and the range between said integers); —(CH2)q-cyclohexyl-(CH2)q-CH3, wherein one or more CH2 are replaced by one or more groups selected from NH and C═O, q is an integer of 0-10 (including 0, 1, 2, 3, 4, 5, 6, 7, 8, 9 or 10, and the range between said integers); —(CH2)r-cyclohexyl-(CH2)r-heteroaryl, wherein one or more CH2 are replaced by one or more groups selected from NH and C═O, r is an integer of 0-10 (including 0, 1, 2, 3, 4, 5, 6, 7, 8, 9 or 10, and the range between said integers), and the heteroaryl has

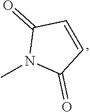

and said —CH3- is optionally substituted by halogen.

In some embodiments, wherein L includes —(CH2)m-, wherein no more than CH2 are replaced by one or more groups selected from NH and C═O, m is an integer of 0-10 (including 0, 1, 2, 3, 4, 5, 6, 7, 8, 9 or 10, and the range between said integers); —(CH2)n-heteroaryl, wherein no more than eight CH2 are replaced by one or more groups selected from NH, C═O and —O—, n is an integer of 0-18 (including 0, 1, 2, 3, 4, 5, 6, 7, 8, 9, 10, 11, 12, 13, 14, 15, 16, 17 or 18, and the range between said integers), and the heteroaryl has

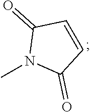

—(CH2)o-piperazinyl-(CH2)o-CH3, wherein one CH2 is replaced by one group selected from NH and C═O, o is an integer of 0-5 (including 0, 1, 2, 3, 4 or 5, and the range between said integers); —(CH2)q-cyclohexyl-(CH2)q-CH3, wherein no more than six CH2 are replaced by one or more groups selected from NH and C═O, q is an integer of 0-8 (including 0, 1, 2, 3, 4, 5, 6, 7 or 8, and the range between said integers); —(CH2)r-cyclohexyl-(CH2)r-heteroaryl, wherein no more than five CH2 are replaced by one or more groups selected from NH and C═O, r is an integer of 0-6 (including 0, 1, 2, 3, 4, 5 or 6, and the range between said integers), and the heteroaryl has

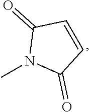

and said —CH3- is optionally substituted by Cl.

In some embodiments, wherein the OPA-L-D is selected from the group consisting of:

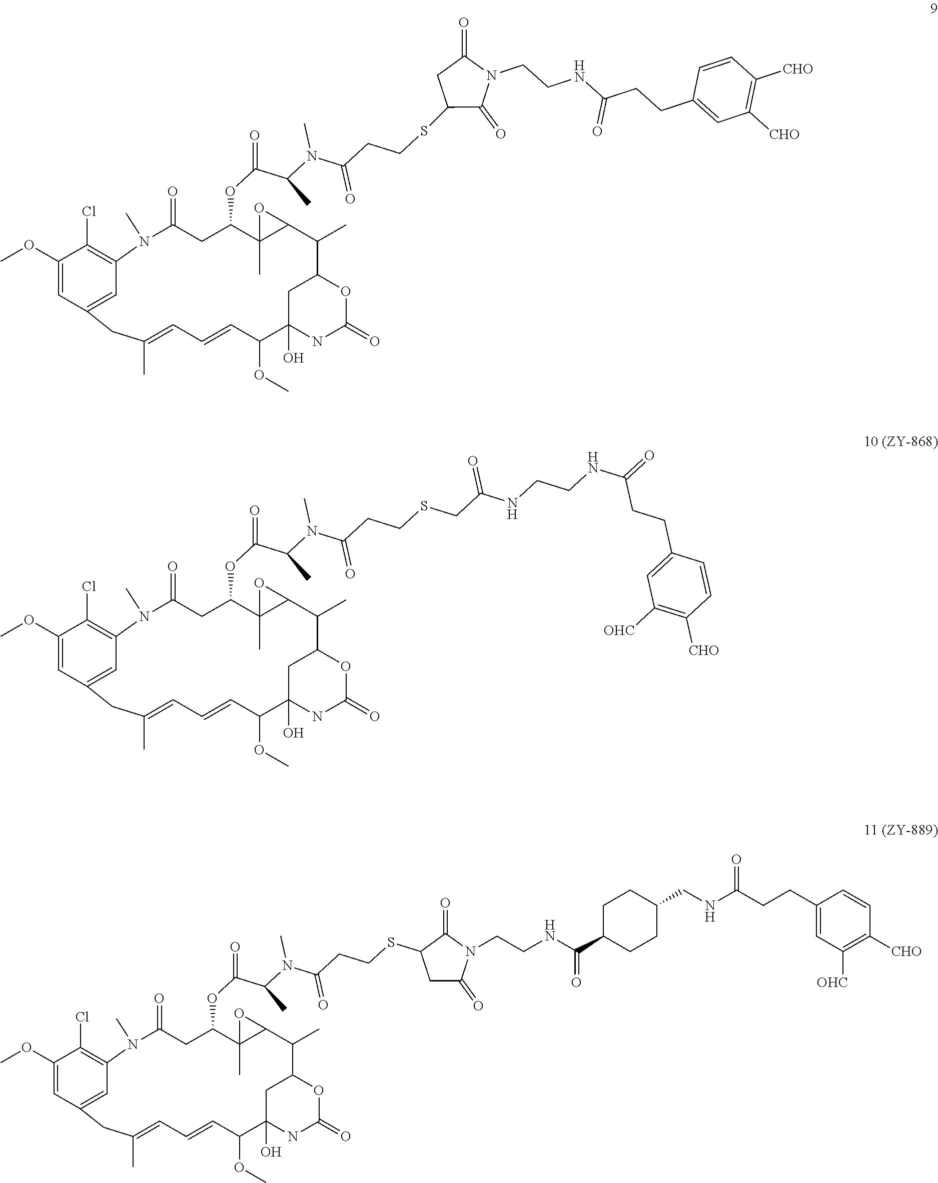 [SMILES: COc1cc2cc(c1Cl)N(C)C(=O)C[C@H](OC(=O)[C@H](C)N(C)C(=O)CCSC1CC(=O)N(CCNC(=O)CCc3ccc(C=O)c(C=O)c3)C1=O)C1(C)OC1C(C)C1CC(O)(NC(=O)O1)C(OC)/C=C/C=C(\C)C2 ; COc1cc2cc(c1Cl)N(C)C(=O)C[C@H](OC(=O)[C@H](C)N(C)C(=O)CCSCC(=O)NCCNC(=O)CCc1ccc(C=O)c(C=O)c1)C1(C)OC1C(C)C1CC(O)(NC(=O)O1)C(OC)/C=C/C=C(\C)C2 ; COc1cc2cc(c1Cl)N(C)C(=O)C[C@H](OC(=O)[C@H](C)N(C)C(=O)CCSC1CC(=O)N(CCNC(=O)[C@H]3CC[C@H](CNC(=O)CCc4ccc(C=O)c(C=O)c4)CC3)C1=O)C1(C)OC1C(C)C1CC(O)(NC(=O)O1)C(OC)/C=C/C=C(\C)C2]

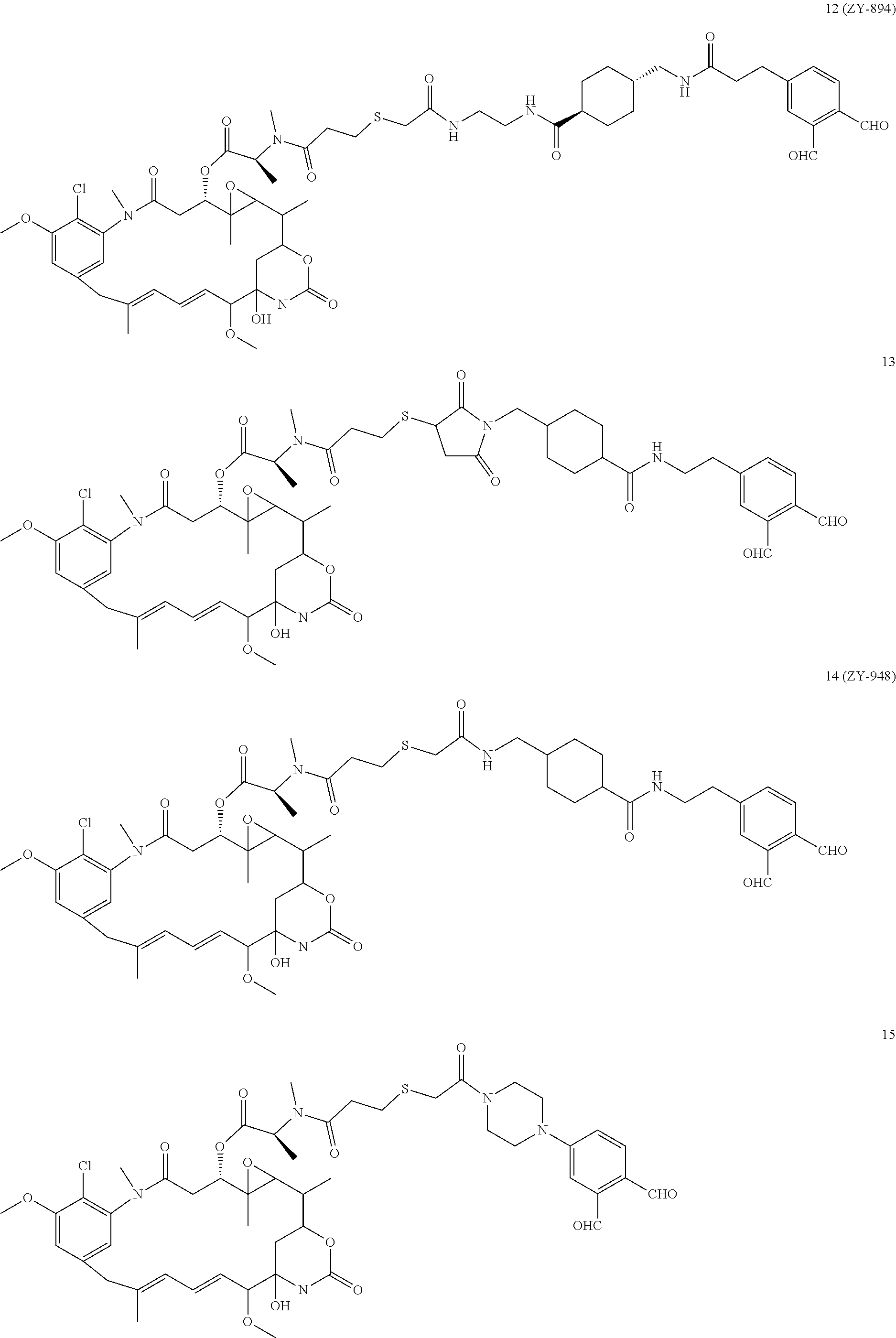

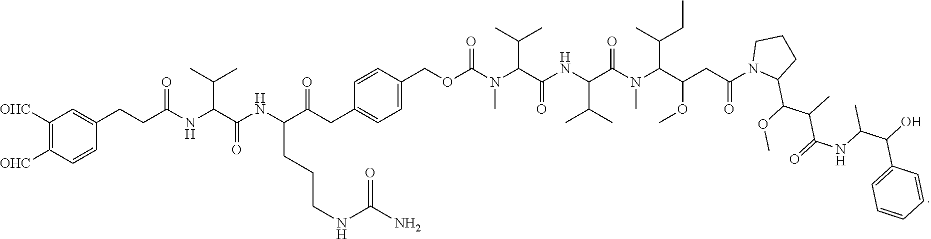 [SMILES: CCC(C)C(C(CC(=O)N1CCCC1C(OC)C(C)C(=O)NC(C)C(O)c1ccccc1)OC)N(C)C(=O)C(NC(=O)C(C(C)C)N(C)C(=O)OCc1ccc(CC(=O)C(CCCNC(N)=O)NC(=O)C(NC(=O)CCc2ccc(C=O)c(C=O)c2)C(C)C)cc1)C(C)C]

In some embodiments, wherein Ab is a cell binding reagent, wherein said cell binding reagent includes IgGs, bi-specific antibody, antibody fragment such as Fab, Fab′, F(ab′)2 and scFv, Heavy-chain only antibody or Nanobody; D is independently active reagent, wherein said active reagent includes an anti-cancer reagent such as Mertansine and MMAE; an anti-inflammation reagent; Fluorescein such as FTIC; a peptide; a protein; a nucleotide; an oligonucleotide; a chemotherapy drug; a natural product; an immune modulator; a tubulin-binder; a DNA-alkylating agent; an HSP90 inhibitor; a DNA topoisomerase inhibitor; an anti-epigenetic agent; an HDAC inhibitor; an anti-metabolism agent; a proteasome inhibitor; a peptidomimetic; an siRNA; an antisense DNA; epothilone A, epothilone B, or paclitaxel; p is a integer refers to the number of active reagent attached to cell binding reagent, wherein p is an integer of 0-15 (including 0, 1, 2, 3, 4, 5, 6, 7, 8, 9, 10, 11, 12, 13, 14 or 15, and the range between said integers).

In some embodiments, said IgGs includes but not limits to IgG 1 (such as, Trastuzumab or Cetuximab), IgG 2, IgG 3, IgG 4 (such as, Inotuzumab) and said Heavy-chain only antibody includes but not limits to HcAb-1 or HcAb-2.

In some embodiments, wherein step (a) is carried out in a buffer (such as, PBS) with pH 7-12 (including pH 7, pH8, pH9, pH10, pH11, pH12 or the range between said pH). Further, the buffer includes but not limits to borate buffer.

In some embodiments, wherein step (b) is carried out in a buffer with pH 4-7 (including pH4, pH5, pH6, pH7 or the range between said pH, such as pH5-7), under 4-37° C. (including 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38° C. or the range between said temperatures) for 1 h-24 h (1, 2, 3, 4, 5, 6, 7, 8, 9, 10, 11, 12, 13, 14, 15, 16, 17, 18, 19, 20, 21, 22, 23, 24 h or the range between said hours, such as 2.5-4 h).

In some embodiments, wherein the buffer in step (b) contains 2.5%-20% (including 2.5%, 3%, 4%, 5%, 6%, 7%, 8%, 9%, 10%, 11%, 12%, 13%, 14%, 15%, 16%, 17%, 18%, 19%, 20% or the range between said contents) organic co-solvent (such as, DMA), and conjugation in step (b) is carried out with 5 eq to 30 eq (5 eq, 6 eq, 7 eq, 8 eq, 9 eq, 10 eq, 11 eq, 12 eq, 13 eq, 14 eq, 15 eq, 16 eq, 17 eq, 18 eq, 19 eq, 20 eq, 21 eq, 22 eq, 23 eq, 24 eq, 25 eq, 26 eq, 27 eq, 28 eq, 29 eq, 30 eq, or the range between said equivalents) of OPA-L-D to Ab.

In some embodiments, wherein an Ab solution with 0.5-15 mg/ml (including 0.5 mg/ml, 1 mg/ml, 2 mg/ml, 3 mg/ml, 4 mg/ml, 5 mg/ml, 6 mg/ml, 7 mg/ml, 8 mg/ml, 9 mg/ml, 10 mg/ml, 11 mg/ml, 12 mg/ml, 13 mg/ml, 14 mg/ml, 15 mg/ml or the range between said concentrations) is used therein.

In some embodiments, to an antibody (Ab) solution with 0.5-15 mg/ml, in buffer with pH 4-7 such as PBS, was added 5-30 eq of OPA containing linker-payload (OPA-L-D) with 2.5%-20% organic co-solvent such as DMA, and the reaction was conducted at 4-37° C. for 1 h-24 h with gentle stirring or shaking. The result product was purified with down-stream steps such as buffer exchange or column purification.

### EXAMPLES

### Example 1. The Synthesis of OPA-L and OPA-L-D

### 1.1. Synthesis Route for 10 (ZY-868)

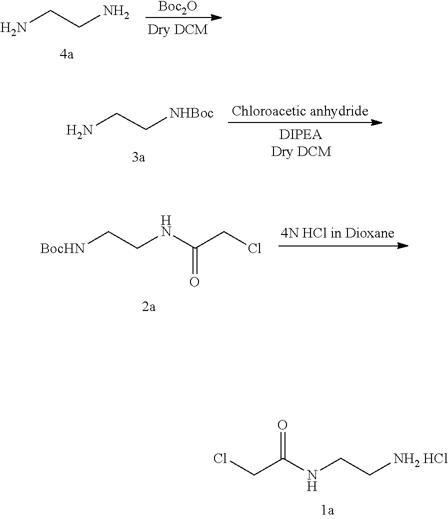

### Tert-butyl (2-aminoethyl)carbamate (3a )

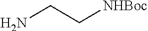 [SMILES: CC(C)(C)OC(=O)NCCN]

1,2-diaminoethane (13.5 mL, 200 mmol) in DCM (200 mL) was cooled in ice bath, Boc-anhydride (4.57 ml, 20 mmol) was dropwise added into reaction over 2 h. After addition was completed, the reaction mixture was stirred at 0° C. for 30 min and then allowed warm to room temperature and stirred for overnight. The reaction was monitored by TLC plate and fully converted, the mixture was then evaporated under vacuo, the residue was dissolved in 200 ml 3N NaCO₃ (aq.) solution. The aqueous solution was extracted twice by DCM, the combine organic layers were dried by anhydrous sodium sulfate, filtered and evaporated under vacuo to obtain pure product 3a as colorless oil (3 g, 94.4%).

### Tert-butyl (2-(2-chloroacetamido)ethyl)carbamate (2a)

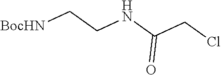 [SMILES: CC(C)(C)OC(=O)NCCNC(=O)CCl]

To a solution of compound 3a (1.4 g, 8.75 mmol) and Et₃N (2.4 ml, 17 mmol) in anhydrous DCM (50 ml) at 0° C., a solution of chloroacetic anhydride (1.79 g, 10.4 mmol) in DCM (8 ml) was dropwise added. The solution was warmed to room temperature and stirred for 2 h. The reaction was monitored by TLC plate and fully converted, the reaction was quenched by 1N HCl solution. The aqueous solution was extracted twice by EtOAc, the organic solution was dried by anhydrous sodium sulfate and evaporated. The residue was purified by flash column chromatography on silica gel (Hexane/EtOAc, 1:1 v/v) to give compound 2a as white solid (1.531 g, 72.8%).

### N-(2-aminoethyl)-2-chloroacetamide hydrochloride (1a)

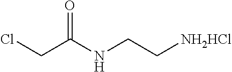

To a solution of compound 2a (1.53 g) in DCM (2 ml), 4N HCl in dioxane solution (5 ml) was added, the reaction was stirred at room temperature for 1 h. The solvent was removed by vacuo and the residue was precipitated by cold Et₂O to give a compound 1a as white solid (1.1 g, 98.2%).

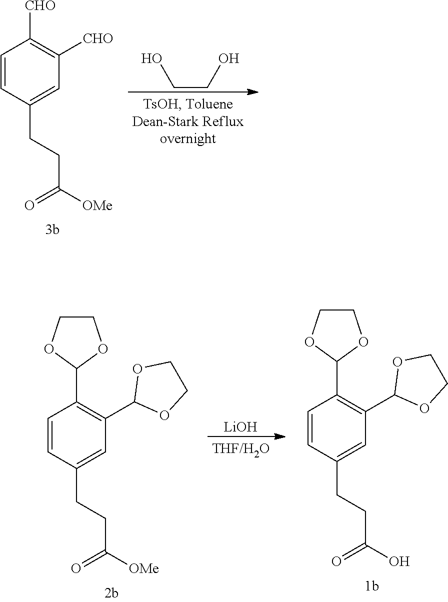

Compound 3b was prepared by literature procedure (See, Tung, C. L.; Wong, C. T. T.; Fung, E. Y. M.; Li, X. Org. Lett. 2016, 18, 2600-2603). To a solution of compound 3b (2.5 g, 9.22 mmol) in anhydrous toluene (200 ml), p-toluenesulfonic acid (32 mg, 0.185 mmol) and ethylene glycol (5.2 ml, 92.15 mmol) were added. The mixture was refluxed in a dean-stark apparatus for overnight. After reaction was cooled down to room temperature, the reaction was quenched by Et₃N (0.8 mL, 5.7 mmol). The mixture was then evaporated under vacuo and the residue was dissolved in EtOAc. The organic layers were washed with Sat. NaHCO₃ (aq.) and brine, dried with anhydrous sodium sulfate and evaporated. The residue was purified by flash column chromatography on silica gel (Hexane/EtOAc, 2:1 v/v) to give compound 2b as yellow oil (2.58 g, 90.5%). To a solution of compound 2b (500 mg, 1.62 mmol) in THF (10 ml), LiOH (113 mg, 4.87 mmol) in water (5 ml) was dropwise added into reaction and stirred at room temperature for 4 h. After acidification with 1 N HCl (aq.), the reaction mixture was extracted by EtOAc, the combined organic layers were washed with brine, dried with anhydrous sodium sulfate and evaporated under vacuo to yield compound 1b as white solid (477 mg, 100%).

### N-(2-(2-chloroacetamido)ethyl)-3-(3,4-di(1,3-dioxolan-2-yl)phenyl)propanamide (3)

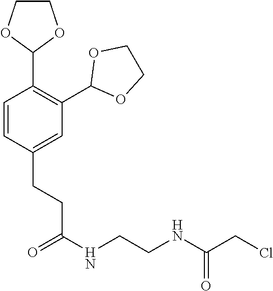 [SMILES: O=C(CCl)NCCNC(=O)CCc1ccc(C2OCCO2)c(C2OCCO2)c1]

To a solution of compound 1b (150 mg, 0.51 mmol) in anhydrous DMF (5 ml), 1-[bis(dimethylamino)methylene]-1H-1,2,3-triazolo[4,5-b]pyridinium 3-oxid hexafluorophosphate (HATU) (194 mg, 0.51 mmol), DIPEA (0.33 mL, 1.8 mmol), compound 1a (150 mg, 0.867 mmol) were added and the reaction was stirred at room temperature for overnight. The reaction was monitored by TLC plate and fully converted, the solvent was removed by vacuo. The residue was dissolved in EtOAc, then washed by 1N HCl solution and brine. The organic layer was dried with anhydrous sodium sulfate and evaporated. The residue was purified by flash column chromatography on silica gel (DCM/EtOAc, 1:2 v/v) to give compound 3 as white solid (183 mg, 89%).

### N-(2-(2-chloroacetamido)ethyl)-3-(3,4-diformylphenyl)propanamide (2)

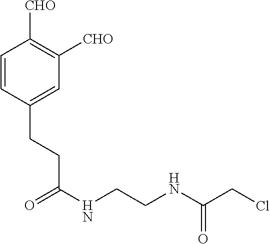 [SMILES: O=Cc1ccc(CCC(=O)NCCNC(=O)CCl)cc1C=O]

To a solution of compound 3 (133 mg, 0.96 mmol) in DCM (2 ml), TFA (8 ml) was slowly added. The reaction was stirred at room temperature for 2 h. The mixture was then evaporated under vacuo, and the residue was purified by flash column chromatography on silica gel (DCM/EtOAc, 1:1 v/v) to give compound 2 as white solid (85 mg, 78%).

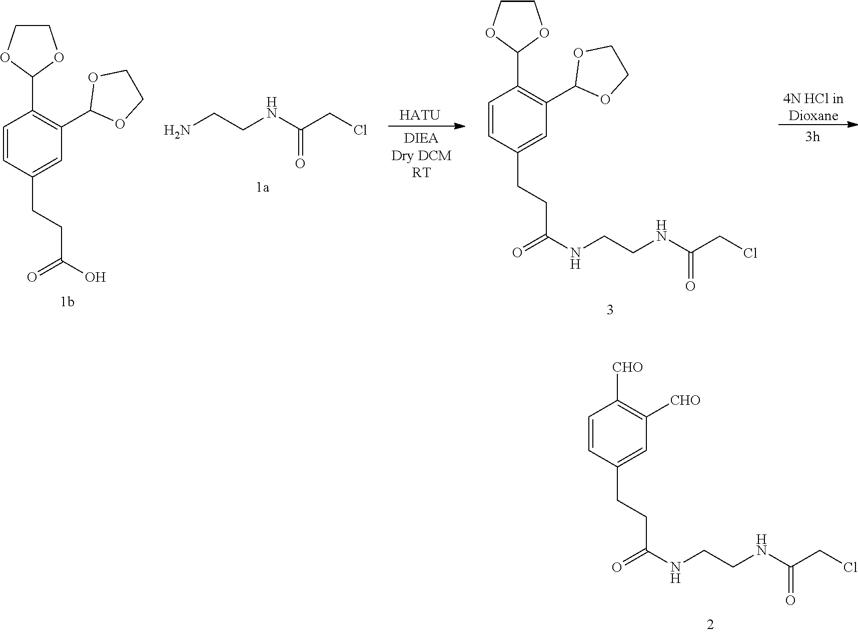

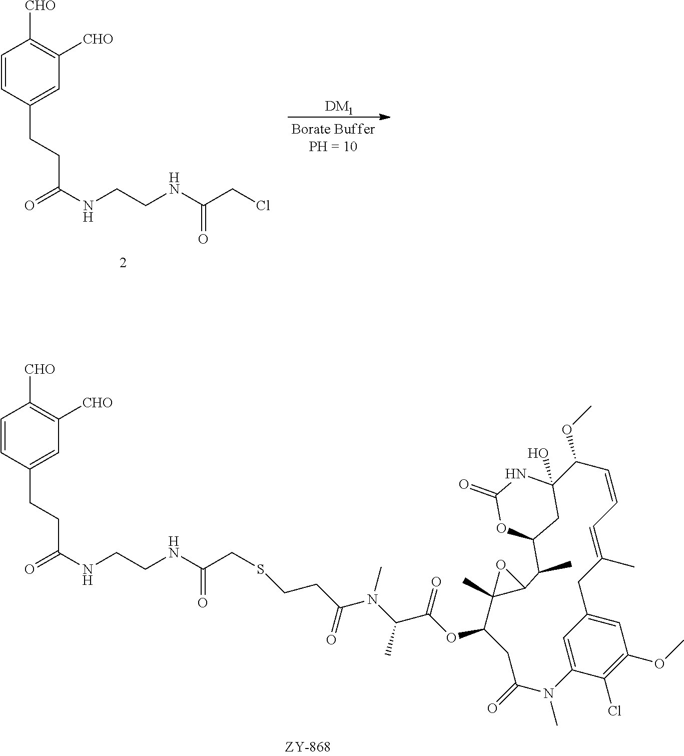

The synthesis started with 2b which was obtained from 3-(4-hydroxyphenyl) propanoic acid by reported method (See, Tung, C. L.; Wong, C. T. T.; Fung, E. Y. M.; Li, X. Org. Lett. 2016, 18, 2600-2603). Compound 2b was first treated with ethylene glycol together with pTsOH to yield compound OPA-COOMe (1b). Next, 2-chloroacetamide compound (1a) was coupled with 1b followed by acidolysis to afford compound 2. Compound 2 was then treated with mertansine in borate buffer (pH=10) to form compound ZY-868.

### (14S,16S,33S,2R,4R,10E,12Z,14R)-86-chloro-4-(((S)-16-(3,4-diformylphenyl)-2,3-dimethyl-4,9,14-trioxo-7-thia-3,10,13-triazahexadecanoyl)oxy)-14-hydroxy-85,14-dimethoxy-33,2,7,10-tetramethyl-12,6-dioxo-7-aza-1(6,4)-oxazinana-3(2,3)-oxirana-8(1,3) benzenacyclotetradecaphane-10,12-dien-9-ylium (ZY-868)

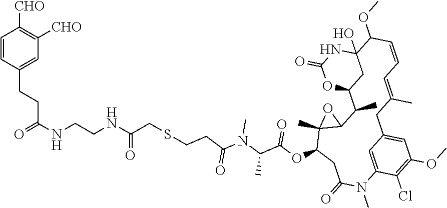 [SMILES: COc1cc2cc(c1Cl)N(C)C(=O)C[C@@H](OC(=O)[C@H](C)N(C)C(=O)CCSCC(=O)NCCNC(=O)CCc1ccc(C=O)c(C=O)c1)[C@]1(C)OC1[C@H](C)[C@@H]1CC(O)(NC(=O)O1)C(OC)/C=C\C=C(/C)C2]

The mertansine (7.38 mg, 0.01 mmol) was dissolved in borate buffer (pH=10) with a final concentration of 0.5 mM. The compound 2 (9.7 mg, 0.029 mmol) in DMSO was slowly added and the reaction was stirred at room temperature for 1 h. The reaction was monitored by RP-LCMS. Preparative HPLC purification (10-60% ACN/H₂O with 0.1% TFA over 45 min) followed by concentration under vacuum and lyophilization afforded ZY-868 as white solid (4.3 mg, 42.57%).

### 1.2. Synthesis Route for 11 (ZY-889)

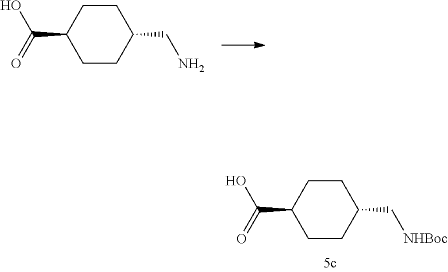 [SMILES: NC[C@H]1CC[C@H](C(=O)O)CC1 ; CC(C)(C)OC(=O)NC[C@H]1CC[C@H](C(=O)O)CC1]

### (1r,4r)-4-(((tert-butoxycarbonyl)amino)methyl)cyclohexane-1-carboxylic acid (5c)

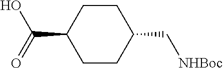 [SMILES: CC(C)(C)OC(=O)NC[C@H]1CC[C@H](C(=O)O)CC1]

To a solution of tranexamic acid (1 g, 6.36 mmol) in anhydrous DCM (50 ml), Et₃N (1.2 ml, 8.6 mmol) and di-tert-butyl dicarbonate (1.6 ml, 6.9 mmol) were added at 0° C. and stirred for overnight. The solvent was removed by vacuo, and the residue was dissolved in water, then acidified by 1N HCl solution and extracted by EtoAc. The organic layer was dried with anhydrous sodium sulfate and evaporated. The residue was purified by flash column chromatography on silica gel to give compound 5c as white (728 mg, 44.6%).

### tert-butyl (((1r,4r)-4-((2-(2,5-dioxocyclopent-3-en-1-yl)ethyl) carbamoyl)cyclohexyl)methyl)carbamate (4c)

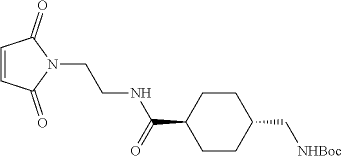 [SMILES: CC(C)(C)OC(=O)NC[C@H]1CC[C@H](C(=O)NCCN2C(=O)C=CC2=O)CC1]

The compound 4c was prepared by following the literature protocol (See, M. Richte, A. Chakrabarti, I. R. Ruttekolk, B. Wiesner, M. Beyermann, R. Brock, J. Rademann, Chem. Eur. J. 2012, 18, 16708-16715). To a solution of compound 5c (728 mg, 2.8 mmol) in anhydrous DMF (6 ml), 1-[bis (dimethylamino) methylene]-1H-1,2,3-triazolo[4,5-b]pyridinium 3-oxid hexafluorophosphate (HATU) (1076 mg, 2.83 mmol), DIPEA (1.73 mL, 9.4 mmol), compound 2-(2-aminoethyl)cyclopent-4-ene-1,3-dione hydrochloride (6c) (750 mg, 4.24 mmol) were added and the reaction was stirred at room temperature for overnight. The reaction was monitored by TLC plate and fully converted, the solvent was removed by vacuo, and the residue was dissolved in EtOAc, then washed by 1N HCl solution and brine. The organic layer was dried with anhydrous sodium sulfate and evaporated. The residue was purified by flash column chromatography on silica gel (Hexane/EtOAc, 1:5 v/v) to give compound 4c as white solid (765 mg, 71.2%).

### (1r,4r)-4-(aminomethyl)-N-(2-(2,5-dioxocyclopent-3-en-1-yl)ethyl)cyclohexane-1-carboxamide hydrochloride (3c)

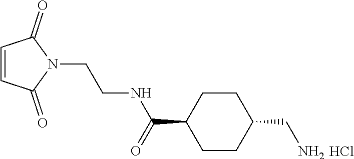 [SMILES: NC[C@H]1CC[C@H](C(=O)NCCN2C(=O)C=CC2=O)CC1 ; Cl]

To a solution of compound 4c (765 mg) in DCM (5 ml), 4N HCl in dioxane solution (8 ml) was dropwise added into the reaction mixture. The mixture was stirred at room temperature for 1 h, the solvent was removed by vacuo and washed the residue with cold Et₂O to give a compound 3c as white solid (732 mg, 100%).

### (1r,4r)-4-((3-(3,4-di(1,3-dioxolan-2-yl)phenyl)propanamido)methyl)-N-(2-(2,5-dioxocyclopent-3-en-1-yl)ethyl)cyclohexane-1-carboxamide (2c)

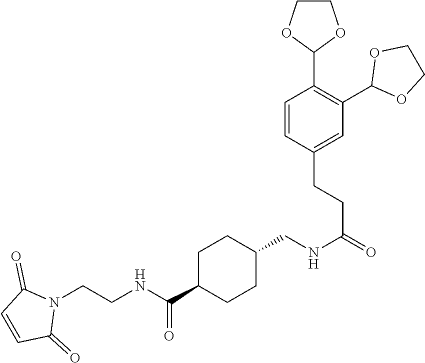 [SMILES: O=C(CCc1ccc(C2OCCO2)c(C2OCCO2)c1)NC[C@H]1CC[C@H](C(=O)NCCN2C(=O)C=CC2=O)CC1]

To a solution of compound 1b (300 mg, 1.02 mmol) in anhydrous DMF 3 ml, 1-[bis(dimethylamino)methylene]-1H-1,2,3-triazolo[4,5-b] pyridinium 3-oxid hexafluorophosphate (HATU) (387.7 mg, 1.02 mmol), DIPEA (0.475 mL, 2.72 mmol), compound 3c (214.8 mg, 0.68 mmol) were added and the reaction was stirred at room temperature for overnight. The reaction was monitored by TLC plate and fully converted, the solvent was removed by vacuo, the residue was dissolved in EtOAc, then washed by brine. The organic layer was dried with anhydrous sodium sulfate and evaporated. The residue was purified by flash column chromatography on silica gel (EtOAc/MeOH, 20:1 v/v) to give compound 2c as white solid (187 mg, 49.7%).

### (1r,4r)-4-((3-(3,4-diformylphenyl)propanamido)methyl)-N-(2-(2,5-dioxocyclopent-3-en-1-yl)ethyl)cyclohexane-1-carboxamide (1c)

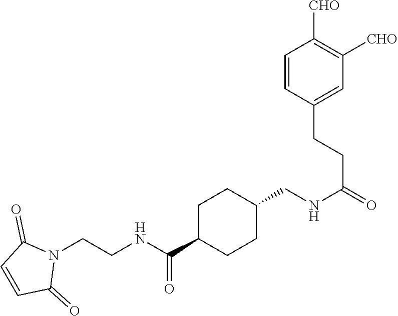 [SMILES: O=Cc1ccc(CCC(=O)NC[C@H]2CC[C@H](C(=O)NCCN3C(=O)C=CC3=O)CC2)cc1C=O]

To a solution of compound 2c (249 mg, 0.44 mmol) in DCM (3 ml), TFA (6 ml) was added into the solution. The mixture was stirred at room temperature for 2 h. The reaction was then evaporated under vacuo, the residue was purified by flash column chromatography on silica gel (DCM/MeOH, 10:1 v/v) to give compound 1c as yellow solid (181.3 mg, 86.5%).

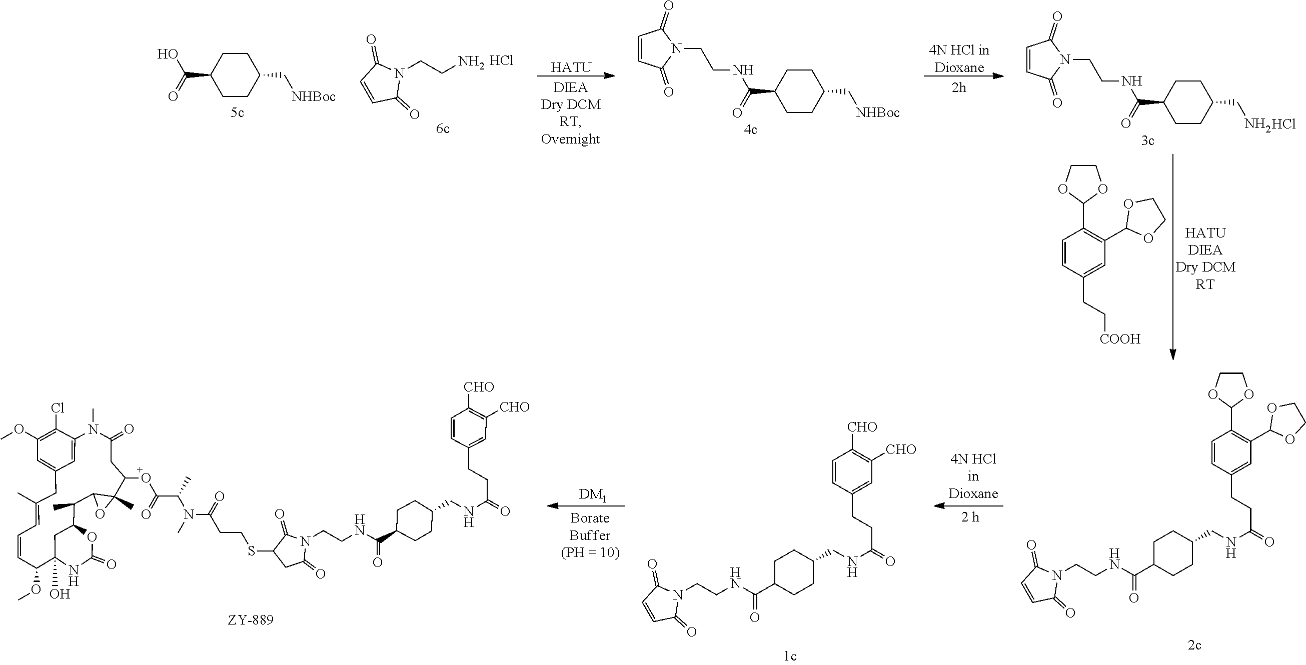

The synthesis started with compound 6c which was obtained from 2-(2-aminoethyl) cyclopent-4-ene-1,3-dione hydrochloride by the reported method (See, M. Richte, A. Chakrabarti, I. R. Ruttekolk, B. Wiesner, M. Beyermann, R. Brock, J. Rademann, Chem. Eur. J. 2012, 18, 16708-16715). Compound 6c was coupled with (1r,4r)-4-(((tert-butoxycarbonyl)amino)methyl)cyclohexane-1-carboxylic acid (5c) to form compound 4c. After Boc removal, the resultant 3c was coupled with OPA-COOH (1b), followed by acidolysis to yield compound 1c. Compound 1c was then reacted with mertansine in borate buffer (pH=10) to form compound ZY-889.

### (14S,16S,33S,2R,4R,10E,12Z,14R)-86-chloro-14-hydroxy-85,14-dimethoxy-33,2,7,10-tetra methyl-12,6-dioxo-7-aza-1(6,4)-oxazinana-3(2,3)-oxirana-8(1,3)-benzenacyclotetradecaphane-10,12-dien-4-yl N-(3-((3-(2-((1r,4r)-4-((3-(3,4-diformylphenyl)propanamido)methyl)cyclohexane-1-carboxamido)ethyl)-2,4-dioxocyclopentyl)thio)propanoyl)-N-methyl-L-alaninate (ZY-889)

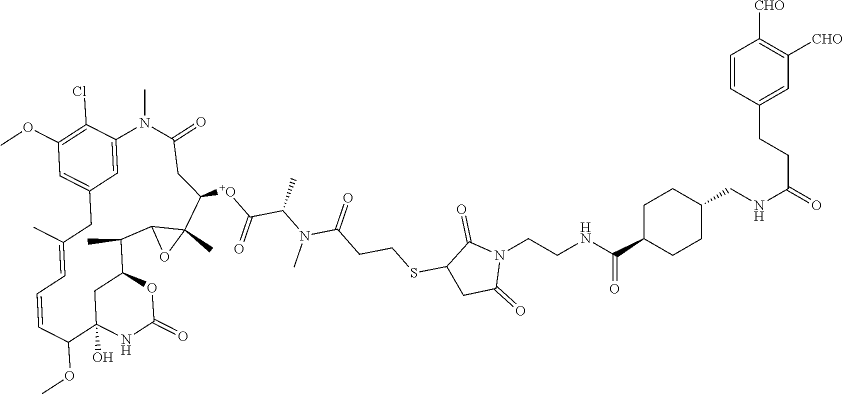 [SMILES: COc1cc2cc(c1Cl)N(C)C(=O)C[C@@H]([OH+]C(=O)[C@H](C)N(C)C(=O)CCSC1CC(=O)N(CCNC(=O)[C@H]3CC[C@H](CNC(=O)CCc4ccc(C=O)c(C=O)c4)CC3)C1=O)[C@]1(C)OC1[C@H](C)[C@@H]1C[C@@](O)(NC(=O)O1)C(OC)/C=C\C=C(/C)C2]

The mertansine (6.642 mg, 0.009 mmol) was dissolved in borate buffer (pH=10) with a final concentration of 0.5 mM. The compound 1c (6.72 mg, 0.014 mmol) in DMSO was slowly added and the mixture was stirred at room temperature for 1 h. The reaction was monitored by RP-LCMS. Preparative HPLC purification (15-55% ACN/H₂O with 0.1% TFA over 45 min) followed by concentration under vacuum and lyophilization afforded ZY-889 (6.25 mg, 59.5%).

### 1.3. Synthesis Route for 12 (ZY-894)

### Methyl (1r,4r)-4-((3-(3,4-di(1,3-dioxolan-2-yl)phenyl)propanamido)methyl) cyclohexane-1-carboxylate (4d)

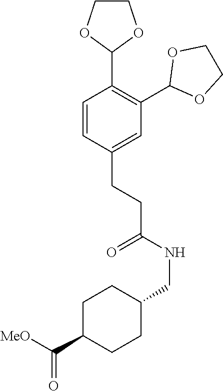 [SMILES: COC(=O)[C@H]1CC[C@H](CNC(=O)CCc2ccc(C3OCCO3)c(C3OCCO3)c2)CC1]

To a stirred solution of compound 1b (100 mg, 0.34 mmol) in anhydrous DMF (2 ml). 1-[Bis(dimethylamino)methylene]-1H-1,2,3-triazolo[4,5-b] pyridinium 3-oxid hexafluorophosphate (HATU) (130 mg, 0.34 mmol), DIPEA (0.119 mL, 0.69 mmol), and trans-Methyl 4-aminomethyl-cyclohexanecarboxylate (117 mg, 0.682 mmol) were added and the mixture was stirred at room temperature for overnight. The reaction was monitored by TLC plate and fully converted, solvent was removed by vacuo, the residue was dissolved in EtOAc, then washed twice by brine. The organic layer was dried with anhydrous sodium sulfate and evaporated under vacuo. The residue was purified by flash column chromatography on silica gel (Hexane/EtOAc, 1:2 v/v) to give compound 4d as white solid (138 mg, 90.1%).

### (1r,4r)-4-((3-(3,4-di(1,3-dioxolan-2-yl)phenyl)propanamido)methyl)cyclohexane-1-carboxylic acid (3d)

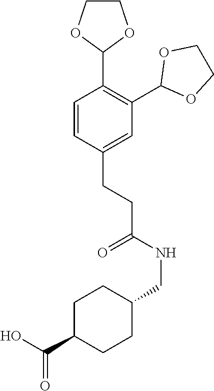 [SMILES: O=C(CCc1ccc(C2OCCO2)c(C2OCCO2)c1)NC[C@H]1CC[C@H](C(=O)O)CC1]

To a stirred solution of compound 4d (137 mg, 0.306 mmol) in THF (9 ml), LiOH (39.45 mg, 0.93 mmol) in water (3 ml) was dropwise added. The mixture was stirred at room temperature for 4 h. After acidification with 1 N HCl (aq.), the reaction mixture was extracted by EtOAc, the combined organic layers were washed with brine, dried with anhydrous sodium sulfate and evaporated. The residue was purified by flash column chromatography on silica gel (EtOAc, 0.1% AcOH) to give compound 3d as white solid (132 mg, 99.1%).

### (1r,4r)-N-(2-(2-chloroacetamido)ethyl)-4-((3-(3,4-di(1,3-dioxolan-2-yl)phenyl)propanamido)methyl)cyclohexane-1-carboxamide (2d)

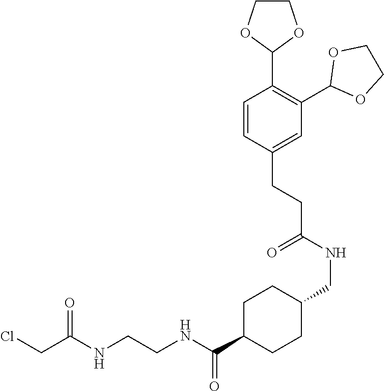 [SMILES: O=C(CCl)NCCNC(=O)[C@H]1CC[C@H](CNC(=O)CCc2ccc(C3OCCO3)c(C3OCCO3)c2)CC1]

To a solution of compound 3d (132 mg, 0.304 mmol) in anhydrous DCM (15 ml). 1-[Bis(dimethylamino)methylene]-1H-1,2,3-triazolo[4,5-b] pyridinium3-oxid hexafluorophosphate (HATU) (116.2 mg, 0.305 mmol), DIPEA (0.213 mL, 1.21 mmol), compound 1a (106 mg, 0.61 mmol) were added and the reaction was stirred at room temperature for overnight. The reaction was monitored by TLC plate and fully converted, solvent was removed by vacuo, the residue was dissolved in EtOAc, then washed by 1N HCl solution and brine. The organic layer was dried with anhydrous sodium sulfate and evaporated. The residue was purified by flash column chromatography on silica gel (EtOAc/MeOH, 15:1 v/v) to give compound 2d as white solid (103 mg, 61.3%).

### (1r,4r)-N-(2-(2-chloroacetamido) ethyl)-4-((3-(3,4 diformylphenyl) propanamido) methyl) cyclohexane-1-carboxamide (1d)

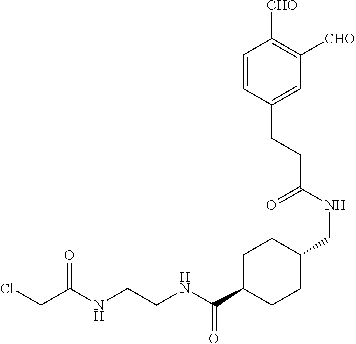 [SMILES: O=Cc1ccc(CCC(=O)NC[C@H]2CC[C@H](C(=O)NCCNC(=O)CCl)CC2)cc1C=O]

To a solution of compound 2d (103 mg, 0.186 mmol) in DCM (4 ml), TFA (6 ml) was slowly added. The reaction was stirred at room temperature for 2 h. The mixture was then evaporated under vacuo, and the residue was purified by flash column chromatography on silica gel (DCM/MeOH, 10:1 v/v) to give compound 1d as white solid (52.9 mg, 60.4%).

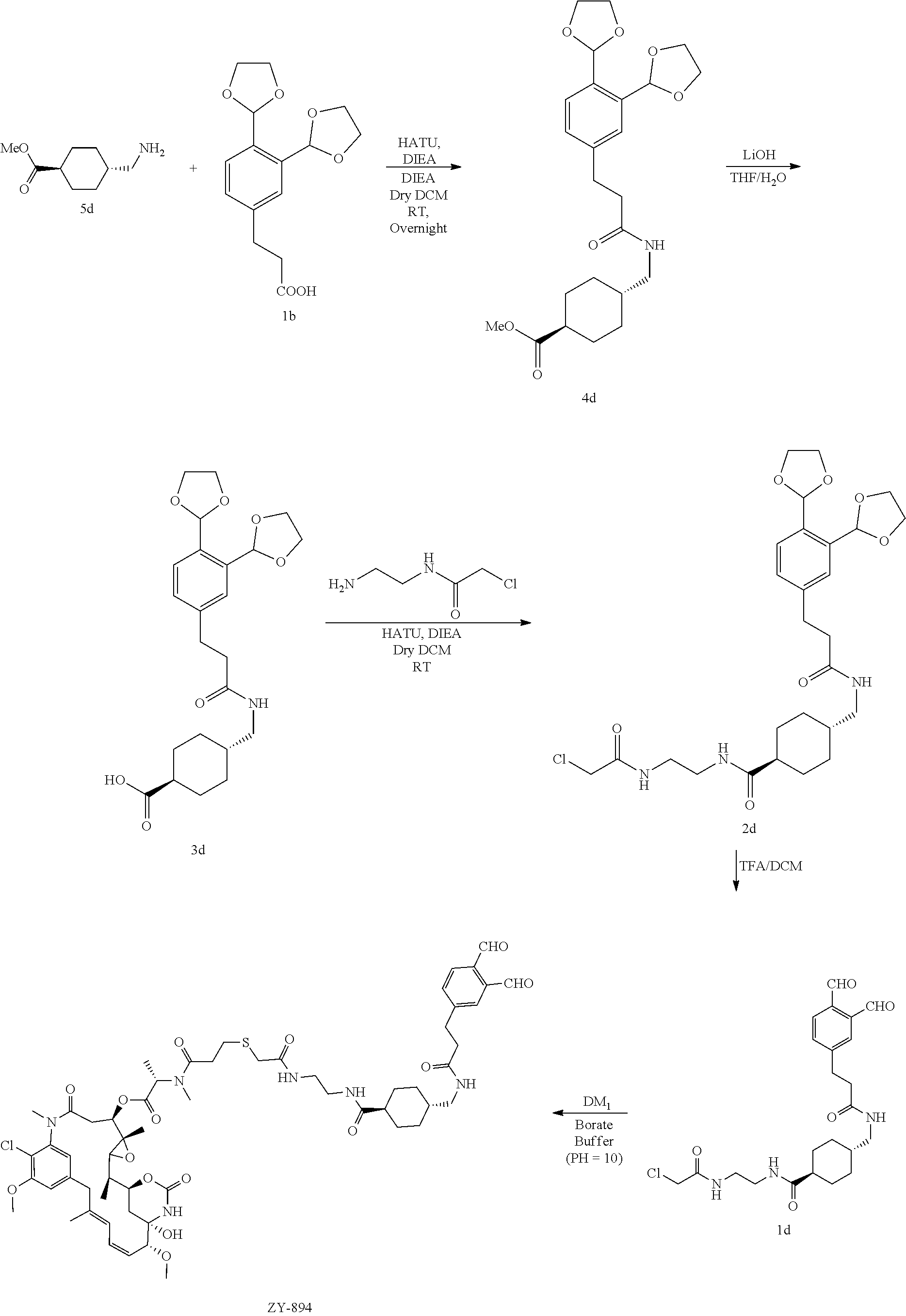

The synthesis started with trans-methyl 4-aminomethyl-cyclohexanecarboxylate (5d) coupled with OPA-COOH (1b) to form compound 4d. After methyl ester de-protection and installation of the a-chloroacetyl group by coupling with compound 1a, compound 2d was obtained. Upon acidolysis, the resultant 1d was reacted with mertansine in borate buffer (pH=10) at room temperature to yield compound ZY-894.

### (14S,16S,33S,2R,4R,10E,12Z,14R)-86-chloro-14-hydroxy-85,14-dimethoxy-33,2,7,10-tetramethyl-12,6-dioxo-7-aza-1(6,4)-oxazinana-3(2,3)-oxirana-8(1,3)-benzenacyclotetradecaphane-10,12-dien-4-yl (13S)-1-((1r,4r)-4-((3-(3,4-diformylphenyl) propanamido) methyl) cyclohexyl)-12,13-dimethyl-1,6,11-trioxo-8-thia-2,5,12-triazatetradecan-14-oate (ZY-894)

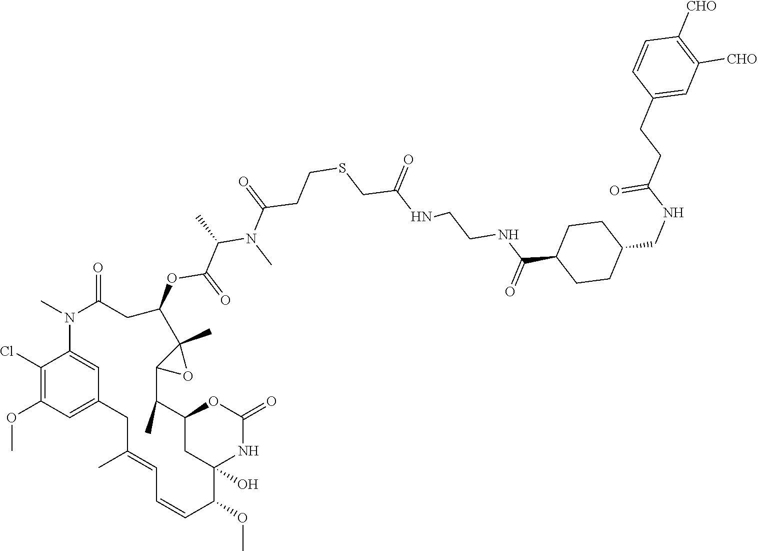 [SMILES: COc1cc2cc(c1Cl)N(C)C(=O)C[C@@H](OC(=O)[C@H](C)N(C)C(=O)CCSCC(=O)NCCNC(=O)[C@H]1CC[C@H](CNC(=O)CCc3ccc(C=O)c(C=O)c3)CC1)[C@]1(C)OC1[C@H](C)[C@@H]1C[C@@](O)(NC(=O)O1)[C@H](OC)/C=C\C=C(/C)C2]

The mertansine (7.011 mg, 0.0095 mmol) was dissolved in borate buffer (pH=10) with a final concentration of 0.5 mM. The compound 1d (8.81 mg, 0.0189 mmol) in DMSO was slowly added and the reaction was stirred at room temperature for 1 h. The reaction was monitored by RP-LCMS. Preparative HPLC purification (10-60% ACN/H₂O with 0.1% TFA over 45 min) followed by concentration under vacuum and lyophilization afforded ZY-894 as white solid (5.15 mg, 47.68%).

### 1.4. Synthesis Route for 14 (ZY-948)

### 4-(2-azidoethyl) phenol (9f)

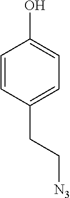 [SMILES: [N-]=[N+]=NCCc1ccc(O)cc1]

To a stirred solution of tyramine (1.5 g, 10.9 mmol) and sodium bicarbonate in anhydrous MeOH (30 ml). Imidazole-1-sulfonyl azide hydrogen sulfate (8.48 g, 31.9 mmol) was added into the mixture at room temperature followed by CuSO₄.5H₂O (7.4 mg, 0.03 mmol). The mixture was stirred at room temperature for overnight. The reaction was monitored by TLC plate and fully converted, the mixture was concentrated, diluted with water (50 mL), acidified with 1N HCl solution and extracted twice with EtOAc. The combined organic layers were washed with brine, dried with anhydrous sodium sulfate and evaporated. The residue was purified by flash column chromatography on silica gel (EtOAc, 0.1% AcOH) to give compound 9f as yellow oil (1.39 g, 78.3%)(See, E. D. G. Borger, R. V. Stick, Org. Lett., 2007, Vol. 9, No. 19, 3797-3800).

### 5-(2-azidoethyl)-2-hydroxybenzaldehyde (8f)

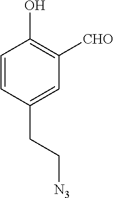 [SMILES: [N-]=[N+]=NCCc1ccc(O)c(C=O)c1]

To a stirred solution of compound 9f (1.39 g, 8.5 mmol), anhydrous magnesium dichloride (1.2 g, 12.6 mmol) and Et₃N (5.45 mL, 39.2 mmol) in anhydrous CH₃CN (70 mL), paraformaldehyde (1.73 g, 57.6 mmol) was added. The reaction mixture was heated under reflux for 3 h and cooled to room temperature. Then acidified by 1N HCl solution and extracted twice with EtOAc (200 ml). The combined organic layer was dried over anhydrous sodium sulfate, filtered, and evaporated. The residue was purified by flash column chromatography on silica gel (Hexane/EtOAc, 2:1 v/v) to give compound 8f as colorless oil (1.29 g, 79.0%).

### 4-(2-azidoethyl) phthalaldehyde (6f)

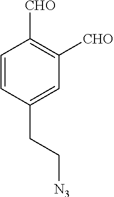 [SMILES: [N-]=[N+]=NCCc1ccc(C=O)c(C=O)c1]

To a stirred solution of compound 8f (1.29 g, 6.74 mmol) in EtOH (40 mL), formic hydrazide (809 mg, 13.4 mmol) in EtOH (40 mL) was slowly added. The reaction mixture was refluxed for 2 h. The reaction was monitored by TLC plate and fully converted, the reaction mixture was cooled in ice bath and the precipitate was filtered and washed with hexane and ice cold EtOH. The residue (7f) was dried under vacuo for overnight. To a stirred solution of compound 7f in anhydrous THF (100 ml), the lead(IV) acetate (6 g, 13.5 mmol) was slowly added. The reaction mixture was then stirred at room temperature for 2.5 h. The reaction was monitored by TLC plate and fully converted, the mixture was filtered through celite, and the filtrate was concentrated under vacuum to give the crude aldehyde. The crude was dissolved in EtOAc (500 ml) and washed by brine. The organic layer was dried over anhydrous sodium sulfate, filtered, and evaporated. The residue was purified by flash column chromatography on silica gel (Hexane/EtOAc, 3; 1 v/v) to give compound 6f as yellow solid (505 mg, 42.1%).

### 2,2′-(4-(2-azidoethyl)-1,2-phenylene)bis(1,3-dioxolane) (5f)

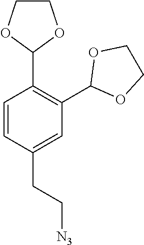 [SMILES: [N-]=[N+]=NCCc1ccc(C2OCCO2)c(C2OCCO2)c1]

To a stirred solution of compound 6f (895 mg, 4.40 mmol) in anhydrous toluene (60 ml), p-toluenesulfonic acid (16 mg, 0.092 mmol) and ethylene glycol (2.5 ml, 44 mmol) were added. The mixture was refluxed in a dean-stark apparatus for overnight. After the mixture was cooled down to room temperature, the reaction was quenched by Et₃N (0.4 mL, 2.85 mmol). The mixture was then evaporated under vacuo, and the residue was dissolved in EtOAc. The organic layers were washed with Sat. NaHCO₃ (aq.) and brine, dried with anhydrous sodium sulfate and evaporated. The residue was purified by flash column chromatography on silica gel (Hexane/EtOAc, 2:1 v/v) to give compound 5f as yellow oil (1.171 g, 91%).

### 2-(3,4-di(1,3-dioxolan-2-yl)phenyl)ethan-1-amine (5)

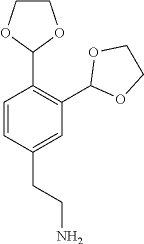 [SMILES: NCCc1ccc(C2OCCO2)c(C2OCCO2)c1]

To a 25 mL round bottom flask, compound 5f (1.17 g, 4.02 mmol) was added, followed by palladium on carbon (10% w/w, 50 mg) and EtOAc (9 ml). The mixture was stirred under 1 atm H₂ atmosphere until the reaction was completed, then was filtered through celite to remove the catalyst. The solvent was concentrated under vacuum and the product 5 (1.01 g, 100%) was obtained as yellow oil.

### (9H-fluoren-9-yl)methyl (((1r,4r)-4-((3,4-di(1,3-dioxolan-2-yl) phenethyl) carbamoyl) cyclohexyl) methyl) carbamate (4e)

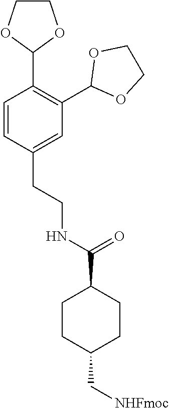

To a stirred solution of (1r,4r)-4-(((((9H-fluoren-9-yl) methoxy) carbonyl) amino) methyl) cyclohexane-1-carboxylic acid (281 mg, 0.740 mmol) in anhydrous DMF (3 ml). N-(3-Dimethylaminopropyl)-N′-ethylcarbodiimide hydrochloride (EDCl) (142 mg, 0.741 mmol), 1-Hydroxybenzotriazole hydrate (HOBt) (100 mg, 0.741 mmol), compound 5 (178.3 mg, 0.672 mmol) and DIEA (0.235 ml, 1.3 mmol) were added and the mixture was stirred at room temperature for overnight. The reaction was monitored by TLC plate and fully converted, solvent was removed by vacuo, the residue was dissolved in EtOAc, then washed by brine. The organic layer was dried with anhydrous sodium sulfate and evaporated. The residue was purified by flash column chromatography on silica gel (Hexane/EtOAc, 1:2 v/v) to give compound 4e as yellow solid (224 mg, 53.2%).

### (1r,4r)-4-((2-chloroacetamido)methyl)-N-(3,4-di(1,3-dioxolan-2-yl)phenethyl)cyclohexane-1-carboxamide (2e)

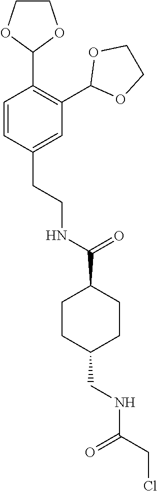 [SMILES: O=C(CCl)NC[C@H]1CC[C@H](C(=O)NCCc2ccc(C3OCCO3)c(C3OCCO3)c2)CC1]

To a stirred solution of compound 4e (73 mg, 0.116 mmol) in DMF/MeCN (3:1), diethylamine (1.4 ml) was added in one portion. The mixture was stirred at room temperature for overnight. The solvent was removed by evaporate under vacuo, the residue was dissolved in anhydrous DCM and cooled in ice bath. The solution of chloroacetic anhydride (91.6 mg, 0.535 mmol) and DIEA (0.4 ml, 0.23 mmol) in DCM (5 ml) was slowly added into reaction mixture, the mixture was stirred at 0° C. to r.t. for 3 h. The reaction was monitored by TLC plate and fully converted, the reaction was quenched by 1N. HCl solution (3 ml). The aqueous solution was extracted twice by EtOAc, the organic solution was dried by anhydrous sodium sulfate and evaporate. The residue was purified by flash column chromatography on silica gel (Hexane/EtOAc, 1:3 v/v) to give compound 2e as white solid (29 g, 33.7%).

### (1r,4r)-4-((2-chloroacetamido)methyl)-N-(3,4-diformylphenethyl)cyclohexane-1-carboxamide (1e)

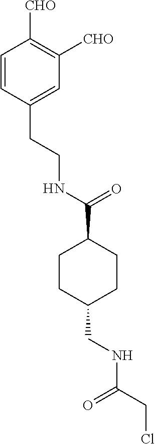 [SMILES: O=Cc1ccc(CCNC(=O)[C@H]2CC[C@H](CNC(=O)CCl)CC2)cc1C=O]

To a 25 mL round bottom flask containing a compound 2e (29 mg, 0.0604 mmol), TFA (5 ml) was added. The mixture was stirred at room temperature for 2 h. The mixture was then evaporated under vacuo, followed by dilute with water and acetonitrile. Preparative HPLC purification (15-50% ACN/H₂O with 0.1% TFA over 45 min) followed by concentration under vacuum and lyophilization afforded 1e as white solid (19.8 mg, 80.5%).

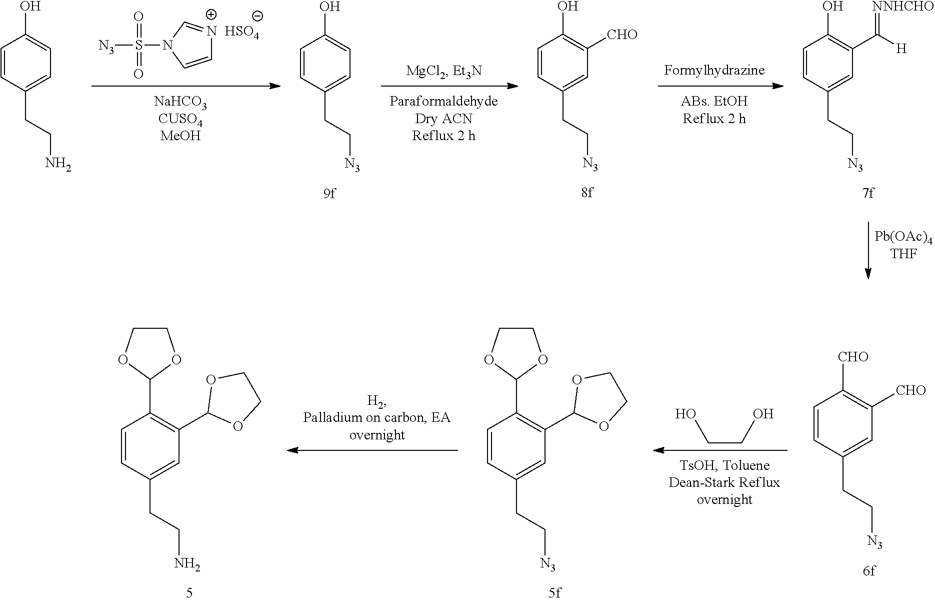

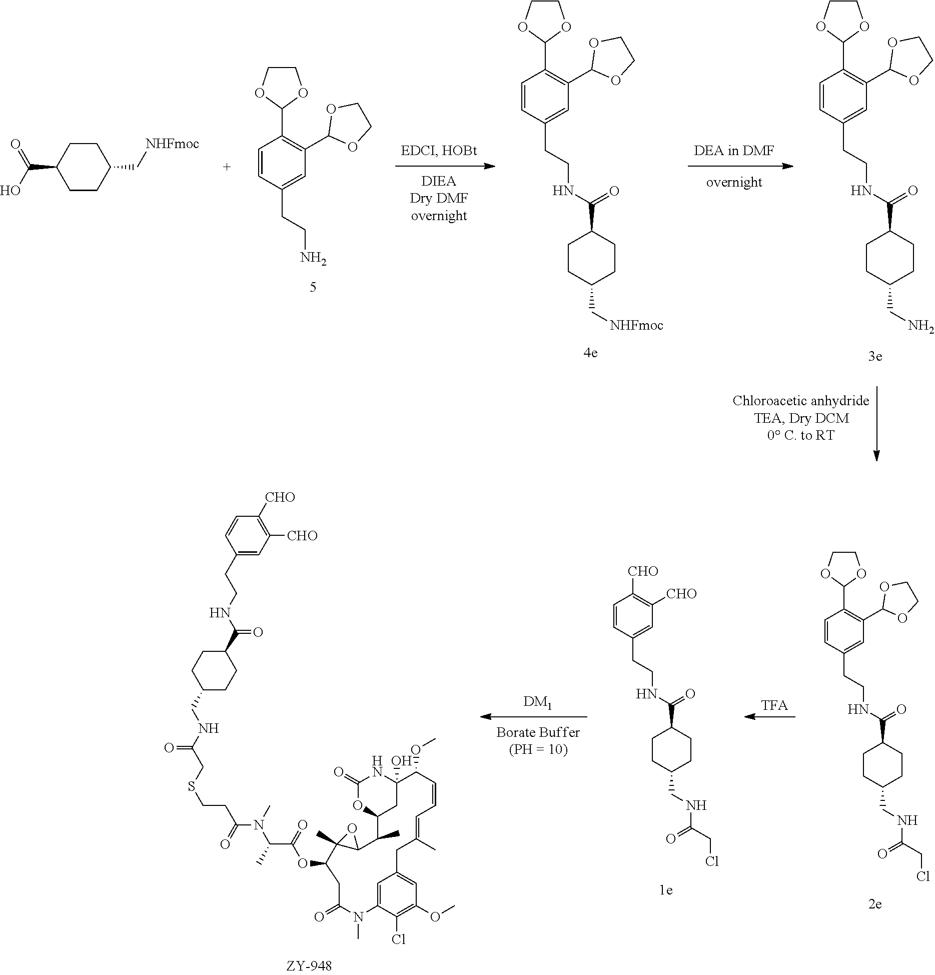

Our synthesis of ZY-948 was started from compound 5, which was obtained from tyramine in 6 steps. Next, compound 5 was coupled with (1r,4r)-4-(((((9H-fluoren-9-yl)methoxy) carbonyl) amino) methyl) cyclohexane-1-carboxylic acid followed by methyl ester de-protection and installation of the a-chloroacetyl group to yield compound 2e. After acidification to yield compound 1e. Compound 1e was then treated with mertansine in borate buffer (pH=10) to form compound ZY-948.

### (14S,16S,33S,2R,4R,10E,12Z,14R)-86-chloro-14-hydroxy-85,14-dimethoxy-33,2,7,10-tetra methyl-12,6-dioxo-7-aza-1(6,4)-oxazinana-3(2,3)-oxirana-8(1,3)-benzenacyclotetradecaphane-10,12-dien-4-yl N-(3-((2-((((1r,4S)-4-((3,4-diformylphenethyl)carbamoyl)cyclohexyl) methyl)amino)-2-oxoethyl)thio)propanoyl)-N-methyl-L-alaninate (ZY-948)

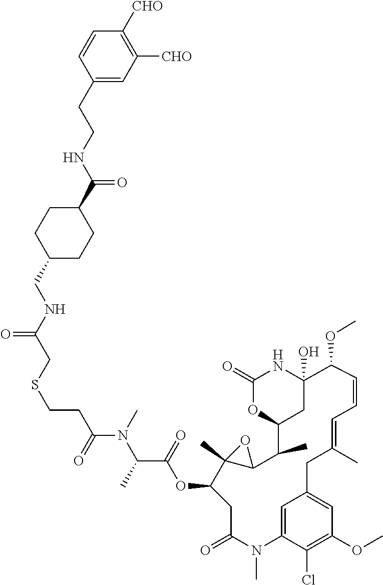 [SMILES: COc1cc2cc(c1Cl)N(C)C(=O)C[C@@H](OC(=O)[C@H](C)N(C)C(=O)CCSCC(=O)NC[C@H]1CC[C@H](C(=O)NCCc3ccc(C=O)c(C=O)c3)CC1)[C@]1(C)OC1[C@H](C)[C@@H]1C[C@@](O)(NC(=O)O1)[C@H](OC)/C=C\C=C(/C)C2]

The mertansine (9.8 mg, 0.0132 mmol) was dissolved in borate buffer (pH=10) with a final concentration of 0.5 mM. The compound 1e (6.27 mg, 0.0159 mmol) in DMSO was slowly added and the reaction was stirred at room temperature for 1 h. The reaction was monitored by RP-LCMS. After reaction was complete. Preparative HPLC purification (15-55% ACN/H₂O with 0.1% TFA over 45 min) followed by concentration under vacuum and lyophilization afforded ZY-948 as white solid (3.1 mg, 21.3%).

### Example 2 the Preparation of Antibody-Drug Conjugate

### Example 2.1 General Conjugation Procedure

To an antibody solution with 0.5-15 mg/ml, in buffer with pH 4-7 such as PBS, was added 5-30 eq of OPA containing linker-payload with 2.5%-20% organic co-solvent such as DMA. The reaction was conducted at 4-37° C. for 1 h-24 h with gentle stirring or shaking. The result product was purified with down-stream steps such as buffer exchange or column purification. For detail, please refer to examples 2.3-2.6. However, a skilled person would understand that the examples herein are not intended to limit the scope of the invention but to illustrate the invention, and the conjugation between other antibody and OPA containing linker-payload (OPA-L-D) can be carried out and completed under the above-general conjugation procedure.

### Example 2.2 General Procedure for the Characterization of Conjugation

The SEC-HPLC parameters are set forth in table 1.

TABLE 1
HPLC parameters

| Equipment | Agilent 1260 series HPLC | |
|---|---|---|
| Column | TSK gel G3000SW_{XL} ((5) 7.8*300, Part NO. 08541) | |
| Column Temp. | 25° | C. |
| Mobile phase | 200 mM KPi, 250 mM KCl, 15% IPA, PH 7.0 | |
| Flow rate | 0.75 | mL/min |
| Sampler Temp. | 4° | C. |
| Injection amount | 30-50 | μg |
| Detection | 280 nm, 252 nm | |
| wavelength | | |
| Stop time | 18.00 | min |

General Procedure of LC-MS for the Characterization of Conjugation

40 μg of the ADC solution was diluted with 5010 storage buffer (Succinate, pH 5.0) and make up to the final volume of 100 μL with 1.0 M Tris-HCl (pH 8.0). 1 μL of PNGaseF (NEB) was added and incubated at 37° C. overnight. Sample was then detected with SEC-HPLC and LC-MS, distribution of each conjugate was calculated by area of each species. Overall DAR is weighted mean value of distribution.

The HPLC and MR parameters are set forth in Tables 2 and 3.

TABLE 2
HPLC Parameters

| Column | Agilent PLRP-S, 1000 Å, 50 × 2.1 mm, 8 μm | | |
|---|---|---|---|
| Detection | 280 & 214 nm | | |
| wavelength | | | |
| Band width | 4 nm | | |
| Column Oven | 80° C. | | |
| Temp. | | | |
| Autosampler | 5° C. | | |
| thermostat | | | |
| Flow Rate | 0.5 mL/min | | |
| Injection Volume | 10 μL | | |
| Mobile Phase | Mobile Phase A, Mobile Phase B | | |
| Gradient | See gradient table below | | |
| Run Time | 10 min | | |
| Gradient Table | Time (min) | % A | % B |
| | 0 | 75 | 25 |
| | 0.7 | 66 | 34 |
| | 5.0 | 55 | 45 |
| | 6.0 | 10 | 90 |
| | 7.0 | 10 | 90 |
| | 7.1 | 75 | 25 |
| | 10.0 | 75 | 25 |

TABLE 3
MS Parameters

| | | |
|---|---|---|
| Gas Temp. | 350° | C. |
| Drying Gas | 13 | L/min |
| Nebulizer | 45 | psig |
| VCap | 5000 | V |
| Fragmentor | 350 | V |
| Mass Range | 500-8000 | m/z |
| Acquisition Rate | 1 | spectra/s |

### Example 2.3 the Preparation of Trastuzumab-ZY-889 Conjugate

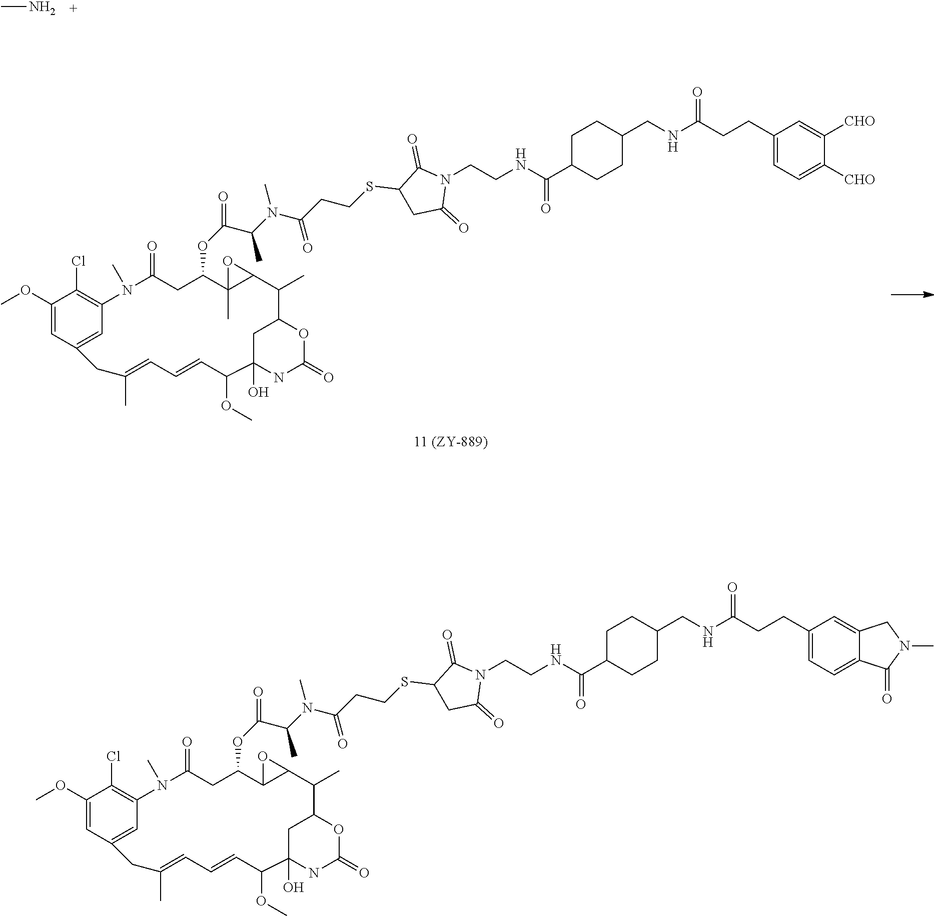

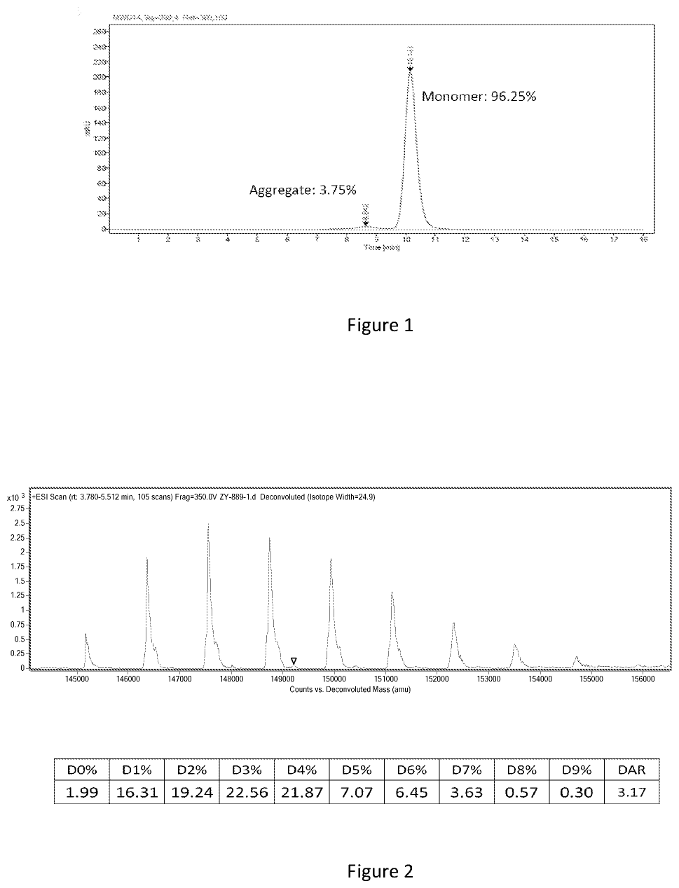

To a solution of 35 mM sodium citrate, 150 mM NaCl, 2 mM EDTA, pH 5.0 was added Trastuzumab as 10 mg/ml. A 6.4 eq of ZY-889 in DMA was added to the antibody solution with final 10% of DMA. The mixture was incubated at 22° C. with gentle shaking for 3 h. The product was purified by 40 KD spin desalting column with buffer exchange to 20 mM succinate, pH 5.0 at the same time. The product of conjugation was characterized by UV-vis, SEC-HPLC and LC-MS. UV-DAR determined by UV-vis was 3.59, SEC-DAR determined by SEC-HPLC was 3.50 and aggregation determined by SEC-HPLC was 3.75% (See, FIG. 1), MS-DAR determined by LC-MS after deglycosylation was 3.17 (See, FIG. 2).

### Example 2.4 the Preparation of Trastuzumab-ZY-948 Conjugate

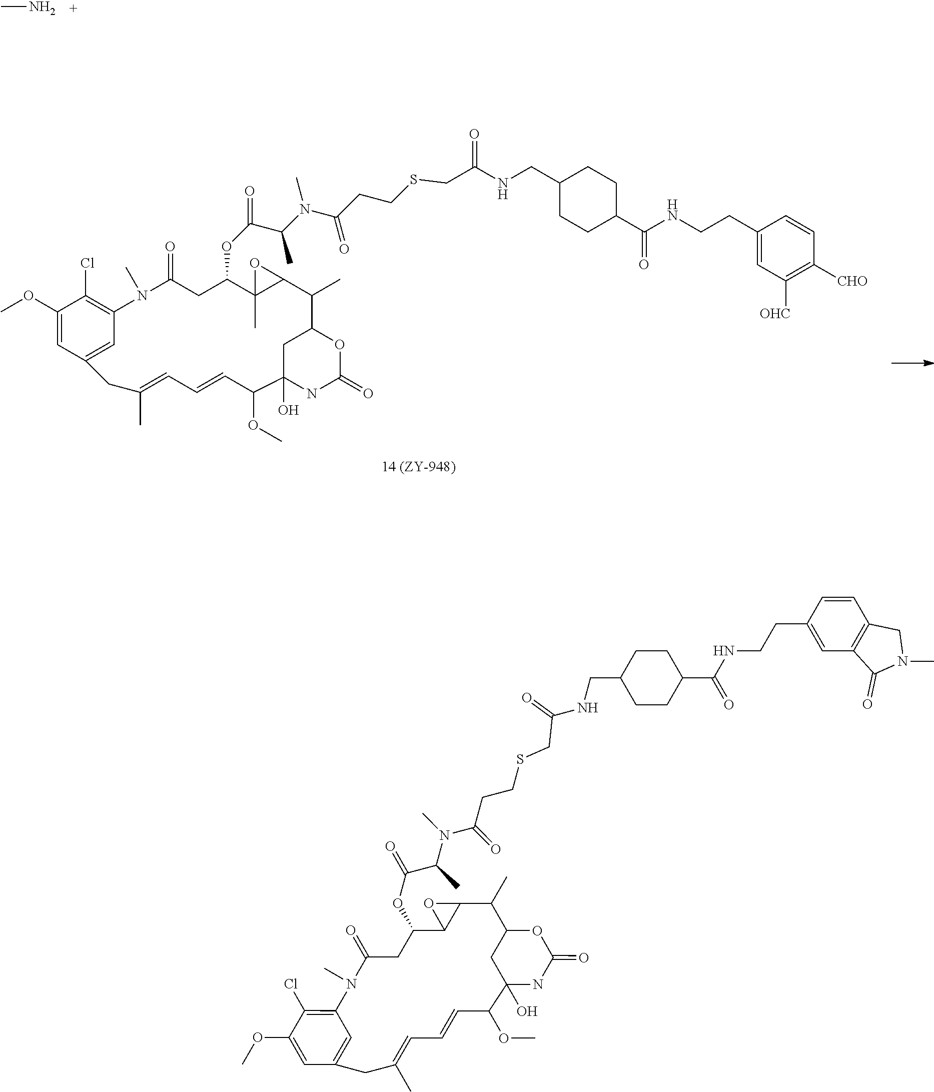

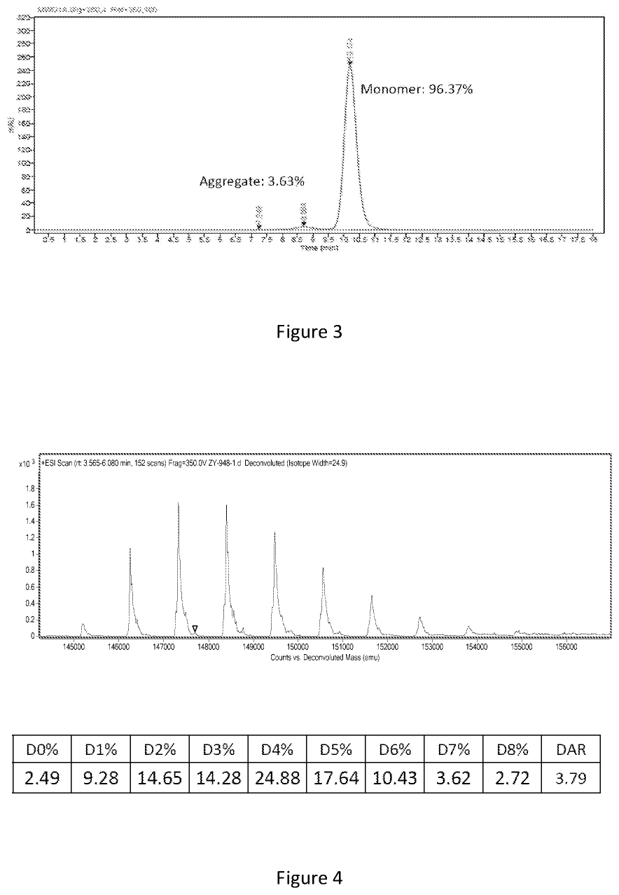

To a solution of 35 mM sodium citrate, 150 mM NaCl, 2 mM EDTA, pH 5.0 was added Trastuzumab as 10 mg/ml. A 9.0 eq of ZY-948 in DMA was added to the antibody solution with final 10% of DMA. The mixture was incubated at 22° C. with gentle shaking for 3 h. The product was purified by 40 KD spin desalting column with buffer exchange to 20 mM succinate, pH 5.0 at the same time. The product of conjugation was characterized by UV-vis, SEC-HPLC and LC-MS. UV-DAR determined by UV-vis was 3.65, SEC-DAR determined by SEC-HPLC was 3.51 and aggregation determined by SEC-HPLC was 3.63% (See, FIG. 3), MS-DAR determined by LC-MS after deglycosylation was 3.79 (See, FIG. 4).

### Example 2.5 the Preparation of HcAb-1-ZY-894 Conjugate

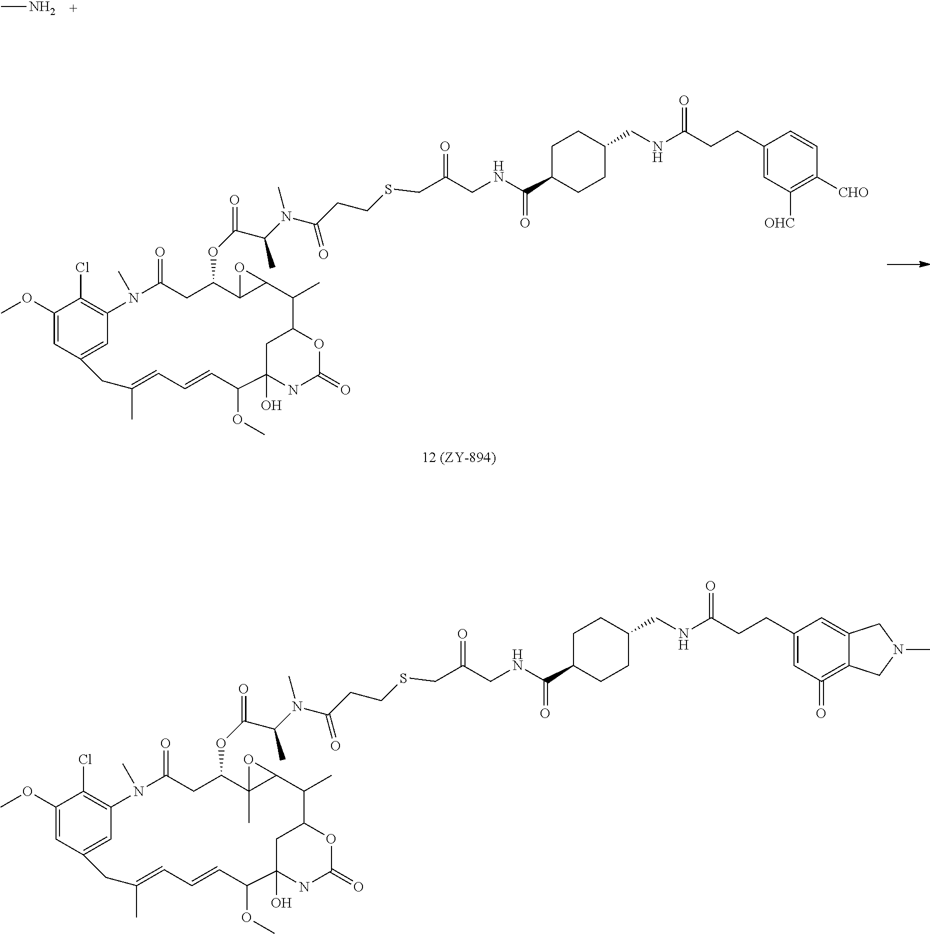

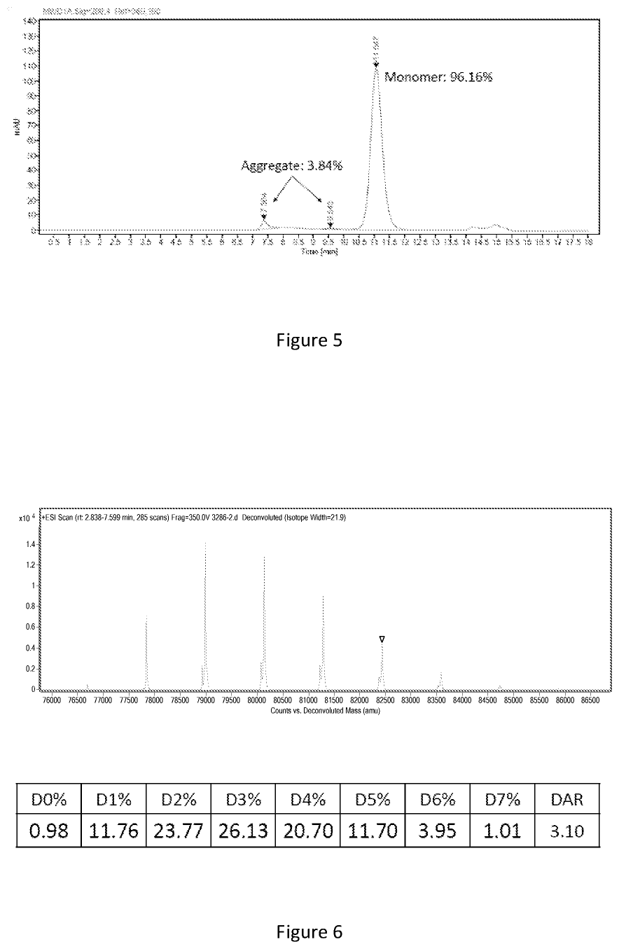

To a solution of 35 mM sodium citrate, 150 mM NaCl, 2 mM EDTA, pH 5.0 was added HcAb-1 (HcAb produce by WuXi Biologics) as 6 mg/ml. A 7.5 eq of ZY-894 in DMA was added to the antibody solution with final 10% of DMA. The mixture was incubated at 22° C. with gentle shaking for 3 h. The product was purified by 40 KD spin desalting column with buffer exchange to 20 mM succinate, pH 5.0 at the same time. The product of conjugation was characterized by UV-vis, SEC-HPLC and LC-MS. UV-DAR determined by UV-vis was 3.78, SEC-DAR determined by SEC-HPLC was 3.73 and aggregation determined by SEC-HPLC was 3.84% (See, FIG. 5), MS-DAR determined by LC-MS after deglycosylation was 3.10 (See, FIG. 6).

### Example 2.6 the Preparation of HcAb-2-ZY-894 Conjugate

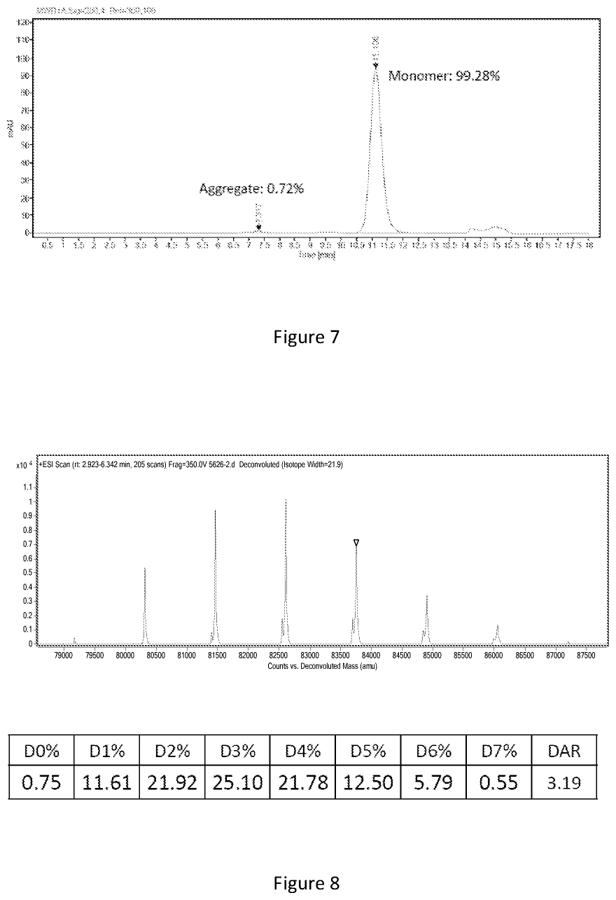

To a solution of 35 mM sodium citrate, 150 mM NaCl, 2 mM EDTA, pH 5.0 was added HcAb-2 (HcAb produce by WuXi Biologics) as 6 mg/ml. A 7.5 eq of ZY-894 in DMA was added to the antibody solution with final 10% of DMA. The mixture was incubated at 22° C. with gentle shaking for 3 h. The product was purified by 40 KD spin desalting column with buffer exchange to 20 mM succinate, pH 5.0 at the same time. The product of conjugation was characterized by UV-vis, SEC-HPLC and LC-MS. UV-DAR determined by UV-vis was 2.70, SEC-DAR determined by SEC-HPLC was 2.78 and aggregation determined by SEC-HPLC was 0.72% (See, FIG. 7), MS-DAR determined by LC-MS after deglycosylation was 3.19 (See, FIG. 8).

### 2.7 Summary of Conjugates with OPA-Containing Linker

Except the conjugates in examples 2.3-2.6, additional conjugates are obtained based on the general conjugation procedure as well. All conjugates obtained (Trastuzumab-ZY-889, Trastuzumab-ZY-948, Trastuzumab-ZY-868, Trastuzumab-ZY-894, Erbitux-ZY-889, Inotuzumab-Zy-889, HcAb-1-ZY-894 and HcAb-2-ZY-894) are summarized in table 4. Of course, a skilled person would understand that the conjugation between other antibody and OPA containing linker-payload (OPA-L-D) can be carried out and completed under the above-general conjugation procedure and other OPA-L containing conjugates also fall into the scope of the invention.

TABLE 4

| | | Conjugation | Reaction | Reaction | Aggregation | | |
|---|---|---|---|---|---|---|---|
| Antibody | Linker | buffer pH | temperature | time | % | UV-DAR | SEC-DAR |
| Trastuzumab | ZY-868 | 5.0 | 22° C. | 2.5 h | 1.43 | 3.53 | 3.54 |
| (IgG1) | | 6.0 | 22° C. | 2.5 h | 2.88 | 3.46 | 3.49 |
| | | 6.5 | 22° C. | 2.5 h | 2.87 | 3.37 | 3.42 |
| | ZY-948 | 5.0 | 22° C. | 3 h | 1.91 | 2.77 | 2.76 |
| | | 5.5 | 22° C. | 3 h | 1.40 | 2.21 | 2.20 |
| | | 6.0 | 22° C. | 3 h | 2.10 | 2.33 | 2.28 |
| | | 6.5 | 22° C. | 3 h | 2.62 | 2.63 | 2.49 |
| | | 5.0 | 22° C. | 4 h | 2.39 | 2.79 | 2.76 |
| | ZY-889 | 5.0 | 22° C. | 2 h | 3.82 | 3.68 | 3.57 |
| | ZY-894 | 5.0 | 22° C. | 2 h | 1.17 | 3.32 | 3.25 |
| | | 7.0 | 22° C. | 2 h | 2.76 | 3.35 | 3.26 |
| | | 7.0 | 4° C. | 2 h | 3.05 | 3.33 | 3.29 |
| | | 7.0 | 22° C. | 3 h | 4.55 | 3.79 | 3.78 |
| | | 7.0 | 4° C. | 3 h | 4.53 | 3.77 | 3.77 |
| Cetuximab | ZY-889 | 5.0 | 22° C. | 3 h | 1.97 | 3.39 | 3.42 |
| (IgG1) | | | | | | | |
| Inotuzumab | ZY-889 | 5.0 | 22° C. | 3 h | 4.42 | 3.23 | 3.25 |
| (IgG4) | | | | | | | |
| HcAb-1 | ZY-894 | 5.0 | 22° C. | 3 h | 3.84 | 3.78 | 3.73 |
| HcAb-2 | ZY-894 | 5.0 | 22° C. | 3 h | 0.72 | 2.70 | 2.78 |

From the data above, we can draw the conclusion that the conjugation reaction can be performed in broad range of buffer components, pH values (5.0-7.0) and reaction temperature (4-22° C.). Meanwhile, the by-product of this reaction is water, thus providing an easy purification after conjugation.

1. A compound of the following formula (I):
OPA-L (I)
Wherein
OPA is 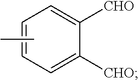
L includes
alkyl, alkenyl, alkynyl, cycloalkyl, cycloalkenyl, cycloalkynyl, heterocycloalkyl, heterocycloalkenyl, heterocycloalkynyl, aryl, heteroaryl, alkylene, alkenylene, alkynylene, arylene, heteroarylene, cycloalkylene, heterocycloalkylene;
—(CH2)m-, wherein one or more CH2 are replaced by one or more groups selected from NH and C═O, m is an integer of 0-20;
—(CH2)n-heteroaryl, wherein one or more CH2 are replaced by one or more groups selected from —NH, —C═O and —O—, n is an integer of 0-20;
—(CH2)o-heterocycloalkyl-(CH2)o-CH3, wherein one or more CH2 are replaced by one or more groups selected from NH and C═O, o is an integer of 0-20;
—(CH2)q-cycloalkyl-(CH2)q-CH3, wherein one or more CH2 are replaced by one or more groups selected from NH and C═O, p is an integer of 0-20;
—(CH2)r-cycloalkyl-(CH2)r-heteroaryl, wherein one or more CH2 are replaced by one or more groups selected from NH and C═O, r is an integer of 0-20;
peptide like di-peptides, tri-peptides, tetra-peptide, penta-peptide;
oligosaccharide, polyethylene glycol (PEG), and the combinations thereof, and
said alkyl, alkenyl, alkynyl, cycloalkyl, cycloalkenyl, cycloalkynyl, heterocycloalkyl, heterocycloalkynyl, aryl, heteroaryl, —CH3, heterocycloalkenyl, alkylene, alkenylene, alkynylene, arylene, heteroarylene, cycloalkylene, heterocycloalkylene are optionally substituted by at least one substituents.

2. The compound according to claim 1, wherein L includes
—(CH2)m-, wherein one or more CH2 are replaced by one or more groups selected from NH and C═O, m is an integer of 0-15;
—(CH2)n-heteroaryl, wherein one or more CH2 are replaced by one or more groups selected from NH, C═O and —O—, n is an integer of 0-20, and the heteroaryl group has 5 to 10 ring members;
—(CH2)o-C₃₋₇heterocycloalkyl-(CH2)o-CH3, wherein one or more CH2 are replaced by one or more groups selected from NH and C═O, o is an integer of 0-15;
—(CH2)q-C₃₋₇cycloalkyl-(CH2)q-CH3, wherein one or more CH2 are replaced by one or more groups selected from NH and C═O, p is an integer of 0-15;
—(CH2)r-C₃₋₇cycloalkyl-(CH2)r-heteroaryl, wherein one or more CH2 are replaced by one or more groups selected from NH and C═O, r is an integer of 0-15, and the heteroaryl group has 5 to 10 ring members, and
said cycloalkyl, heterocycloalkyl, —CH3 and heteroaryl are optionally substituted by oxo and halogen.

3. The compound according to claim 2, wherein L includes
—(CH2)m-, wherein one or more CH2 are replaced by one or more groups selected from NH and C═O, m is an integer of 0-10;
—(CH2)n-heteroaryl, wherein one or more CH2 are replaced by one or more groups selected from NH, C═O and —O—, n is an integer of 0-20, and the 5 to 10 membered heteroaryl has 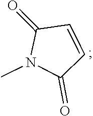
—(CH2)o-piperazinyl-(CH2)o-CH3, wherein one or more CH2 are replaced by one or more groups selected from NH and C═O, o is an integer of 0-10;
—(CH2)q-cyclohexyl-(CH2)q-CH3, wherein one or more CH2 are replaced by one or more groups selected from NH and C═O, p is an integer of 0-10;
—(CH2)r-cyclohexyl-(CH2)r-heteroaryl, wherein one or more CH2 are replaced by one or more groups selected from NH and C═O, r is an integer of 0-10, and the 5 to 10 membered heteroaryl has 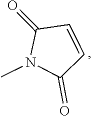
and
said —CH3- is optionally substituted by halogen.

4. The compound according to claim 3, wherein L includes
—(CH2)m-, wherein one or more CH2 are replaced by one or more groups selected from NH and C═O, m is an integer of 0-10;
—(CH2)n-heteroaryl wherein no more than eight CH2 are replaced by one or more groups selected from NH, C═O and —O—, n is an integer of 0-18, and the 5 to 10 membered heteroaryl has 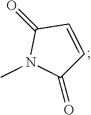
—(CH2)o-piperazinyl-(CH2)o-CH3, wherein one CH2 is replaced by one group selected from NH and C═O, o is an integer of 0-5;
—(CH2)q-cyclohexyl-(CH2)q-CH3, wherein no more than six CH2 are replaced by one or more groups selected from NH and C═O, p is an integer of 0-8;
—(CH2)r-cyclohexyl-(CH2)r-heteroaryl, wherein no more than five CH2 are replaced by one or more groups selected from NH and C═O, r is an integer of 0-6, and the 5 to 10 membered heteroaryl has 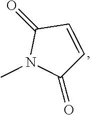
and
said —CH3- is optionally substituted by Cl.

5. The compound according to claim 4, wherein the compound is selected from the group consisting of:

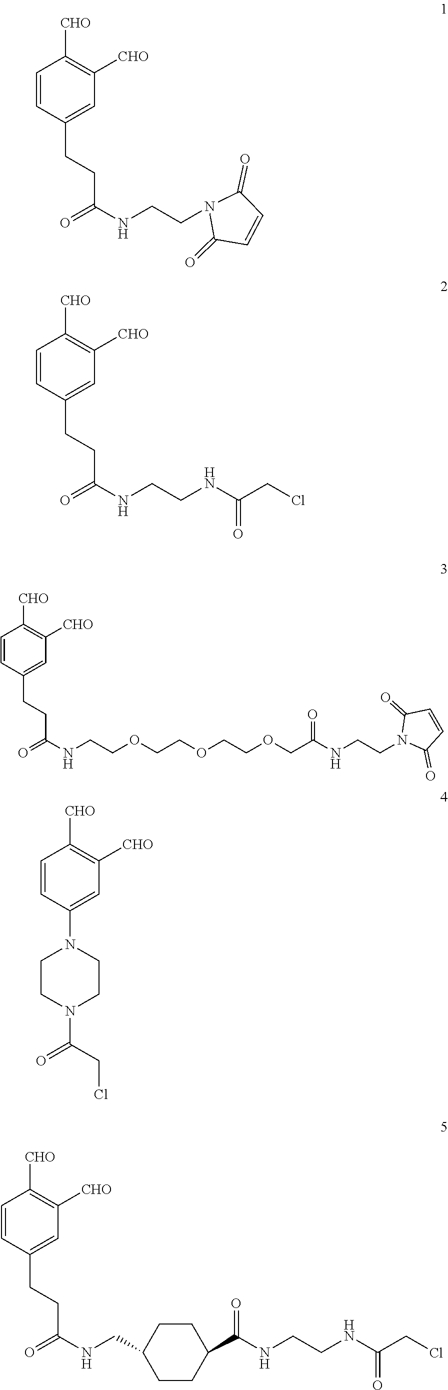 [SMILES: O=Cc1ccc(CCC(=O)NCCN2C(=O)C=CC2=O)cc1C=O ; O=Cc1ccc(CCC(=O)NCCNC(=O)CCl)cc1C=O ; O=Cc1ccc(CCC(=O)NCCOCCOCCOCC(=O)NCCN2C(=O)C=CC2=O)cc1C=O ; O=Cc1ccc(N2CCN(C(=O)CCl)CC2)cc1C=O ; O=Cc1ccc(CCC(=O)NC[C@H]2CC[C@H](C(=O)NCCNC(=O)CCl)CC2)cc1C=O]

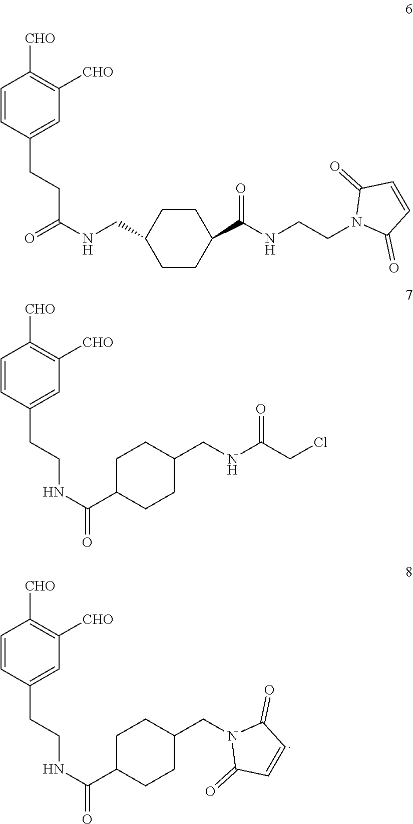 [SMILES: O=Cc1ccc(CCC(=O)NC[C@H]2CC[C@H](C(=O)NCCN3C(=O)C=CC3=O)CC2)cc1C=O ; O=Cc1ccc(CCNC(=O)C2CCC(CNC(=O)CCl)CC2)cc1C=O ; O=Cc1ccc(CCNC(=O)C2CCC(CN3C(=O)C=CC3=O)CC2)cc1C=O]

6. A compound of the following formula (II):
OPA-L-D (II)
Wherein
OPA is 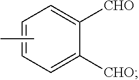
L includes alkyl, alkenyl, alkynyl, cycloalkyl, cycloalkenyl, cycloalkynyl, heterocycloalkyl,
heterocycloalkenyl, heterocycloalkynyl, aryl, heteroaryl, alkylene, alkenylene, alkynylene, arylene, heteroarylene, cycloalkylene, heterocycloalkylene; —(CH2)m-, wherein one or more CH2 are replaced by one or more groups selected from NH and C═O, m is an integer of 0-20; —(CH2)n-heteroaryl, wherein one or more CH2 are replaced by one or more groups selected from NH, C═O and —O—, n is an integer of 0-20; —(CH2)o-heterocycloalkyl-(CH2)o-CH3, wherein one or more CH2 are replaced by one or more groups selected from NH and C═O, o is an integer of 0-20; —(CH2)q-cycloalkyl-(CH2)q-CH3, wherein one or more CH2 are replaced by one or more groups selected from NH and C═O, p is an integer of 0-20; —(CH2)r-cycloalkyl-(CH2)r-heteroaryl, wherein one or more CH2 are replaced by one or more groups selected from NH and C═O, r is an integer of 0-20; peptide; oligosaccharide, polyethylene glycol (PEG), and the combinations thereof, and said alkyl, alkenyl, alkynyl, cycloalkyl, cycloalkenyl, cycloalkynyl, heterocycloalkyl, heterocycloalkynyl, aryl, heteroaryl, —CH3, heterocycloalkenyl, alkylene, alkenylene, alkynylene, arylene, heteroarylene, cycloalkylene, heterocycloalkylene are optionally substituted by at least one substituents;
D is independently active reagent, wherein said active reagent includes an anti-cancer reagent such as Mertansine and MMAE; an anti-inflammation reagent; Fluorescein such as FTIC; a peptide; a protein; a nucleotide; an oligonucleotide; a chemotherapy drug; a natural product; an immune modulator; a tubulin-binder; a DNA-alkylating agent; an HSP90 inhibitor; a DNA topoisomerase inhibitor; an anti-epigenetic agent; an HDAC inhibitor; an anti-metabolism agent;
a proteasome inhibitor; a peptidomimetic; an siRNA; an antisense DNA; epothilone A, epothilone B, or paclitaxel.

7. The compound according to claim 6, wherein L includes
—(CH2)m-, wherein one or more CH2 are replaced by one or more groups selected from NH and C═O, m is an integer of 0-15;
—(CH2)n-heteroaryl, wherein one or more CH2 are replaced by one or more groups selected from NH, C═O and —O—, n is an integer of 0-20, and the heteroaryl group has 5 to 10 ring members;
—(CH2)o-C₃₋₇heterocycloalkyl-(CH2)o-CH3, wherein one or more CH2 are replaced by one or more groups selected from NH and C═O, o is an integer of 0-15;
—(CH2)q-C₃₋₇cycloalkyl-(CH2)q-CH3, wherein one or more CH2 are replaced by one or more groups selected from NH and C═O, q is an integer of 0-15;
—(CH2)r-C₃₋₇cycloalkyl-(CH2)r-heteroaryl, wherein one or more CH2 are replaced by one or more groups selected from NH and C═O, r is an integer of 0-15, and the heteroaryl group has 5 to 10 ring members, and
said cycloalkyl, heterocycloalkyl, —CH3 and heteroaryl are optionally substituted by oxo and halogen.

8. The compound according to claim 7, wherein L includes
—(CH2)m-, wherein one or more CH2 are replaced by one or more groups selected from NH and C═O, m is an integer of 0-10;
—(CH2)n-heteroaryl, wherein one or more CH2 are replaced by one or more groups selected from NH, C═O and —O—, n is an integer of 0-20, and the 5 to 10 membered heteroaryl has 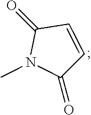
—(CH2)o-piperazinyl-(CH2)o-CH3, wherein one or more CH2 are replaced by one or more groups selected from NH and C═O, o is an integer of 0-10;
—(CH2)q-cyclohexyl-(CH2)q-CH3, wherein one or more CH2 are replaced by one or more groups selected from NH and C═O, q is an integer of 0-10;
—(CH2)r-cyclohexyl-(CH2)r-heteroaryl, wherein one or more CH2 are replaced by one or more groups selected from NH and C═O, r is an integer of 0-10, and the 5 to 10 membered heteroaryl has 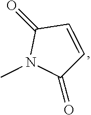
and
said —CH3- is optionally substituted by halogen.

9. The compound according to claim 8, wherein L includes
—(CH2)m-, wherein one or more CH2 are replaced by one or more groups selected from NH and C═O, m is an integer of 0-10;
—(CH2)n-heteroaryl, wherein no more than eight CH2 are replaced by one or more groups selected from NH, C═O and —O—, n is an integer of 0-18, and the 5 to 10 membered heteroaryl has 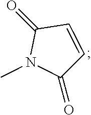
—(CH2)o-piperazinyl-(CH2)o-CH3, wherein one CH2 is replaced by one group selected from NH and C═O, o is an integer of 0-5;
—(CH2)q-cyclohexyl-(CH2)q-CH3, wherein no more than six CH2 are replaced by one or more groups selected from NH and C═O, q is an integer of 0-8;
—(CH2)r-cyclohexyl-(CH2)r-heteroaryl, wherein no more than five CH2 are replaced by one or more groups selected from NH and C═O, r is an integer of 0-6, and the 5 to 10 membered heteroaryl has 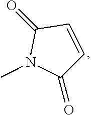
and
said —CH3- is optionally substituted by Cl.

10. The compound according to claim 9, wherein the compound is selected from the group consisting of:

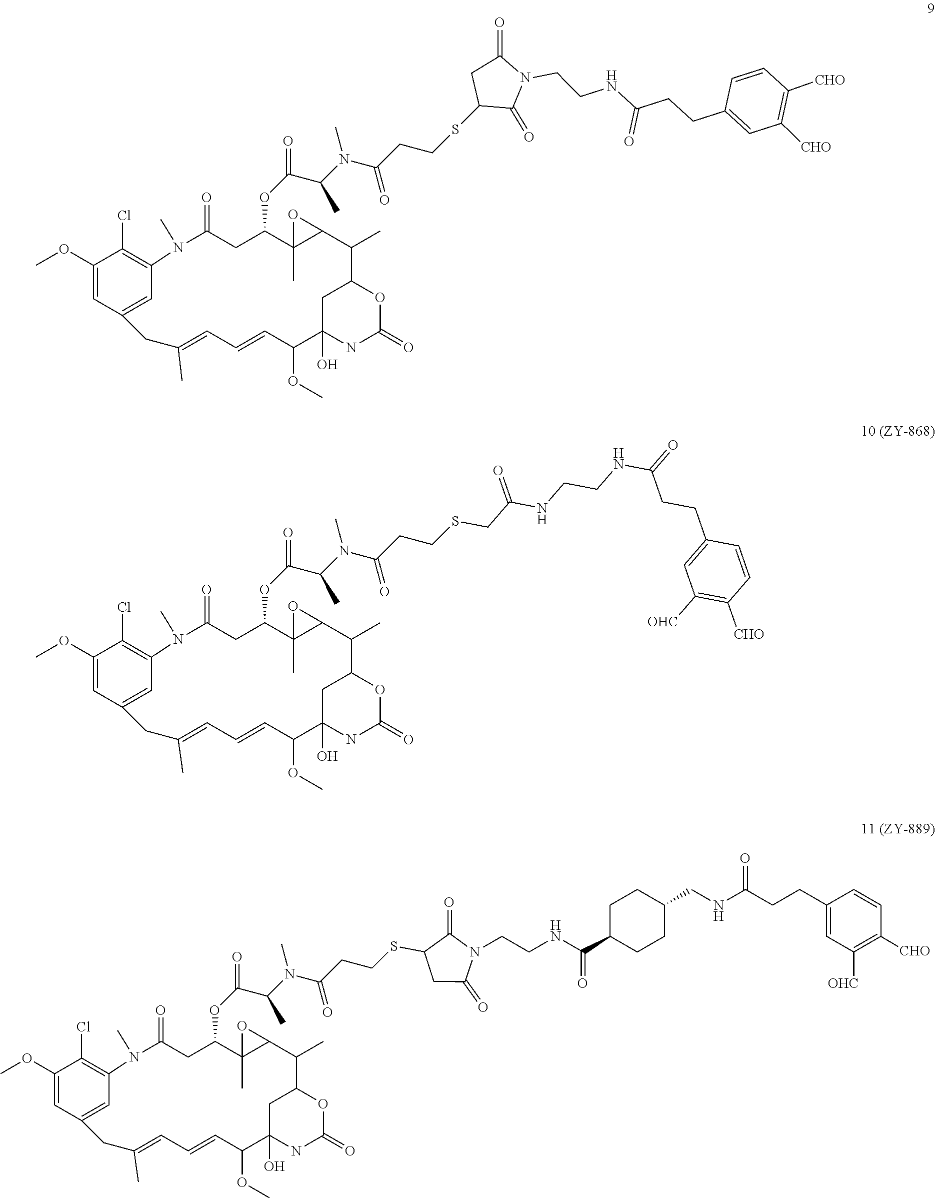 [SMILES: COc1cc2cc(c1Cl)N(C)C(=O)C[C@H](OC(=O)[C@H](C)N(C)C(=O)CCSC1CC(=O)N(CCNC(=O)CCc3ccc(C=O)c(C=O)c3)C1=O)C1(C)OC1C(C)C1CC(O)(NC(=O)O1)C(OC)/C=C/C=C(\C)C2 ; COc1cc2cc(c1Cl)N(C)C(=O)C[C@H](OC(=O)[C@H](C)N(C)C(=O)CCSCC(=O)NCCNC(=O)CCc1ccc(C=O)c(C=O)c1)C1(C)OC1C(C)C1CC(O)(NC(=O)O1)C(OC)/C=C/C=C(\C)C2 ; COc1cc2cc(c1Cl)N(C)C(=O)C[C@H](OC(=O)[C@H](C)N(C)C(=O)CCSC1CC(=O)N(CCNC(=O)[C@H]3CC[C@H](CNC(=O)CCc4ccc(C=O)c(C=O)c4)CC3)C1=O)C1(C)OC1C(C)C1CC(O)(NC(=O)O1)C(OC)/C=C/C=C(\C)C2]

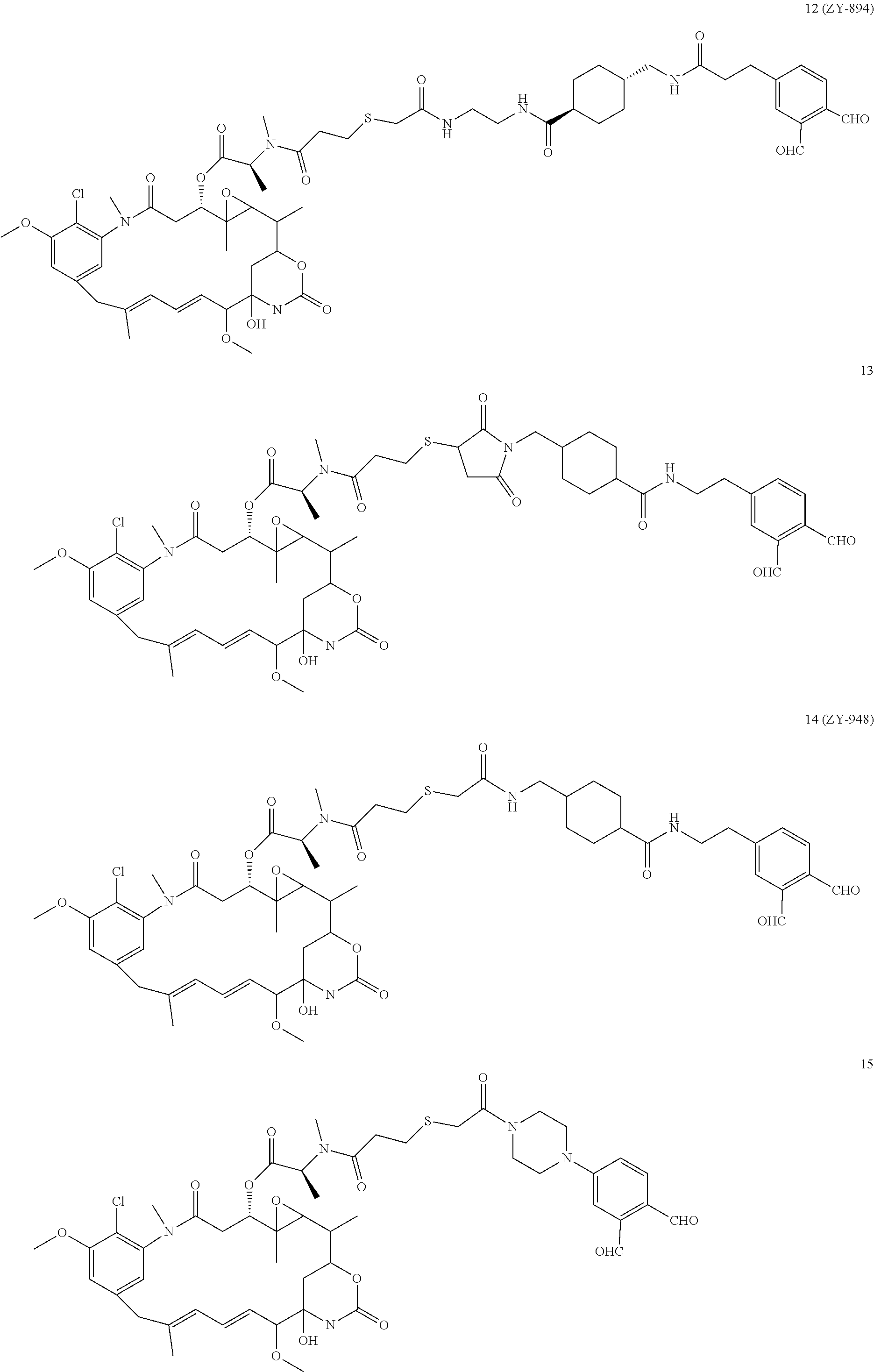

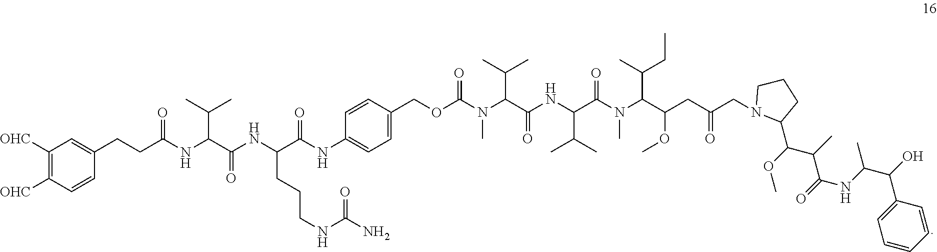 [SMILES: CCC(C)C(C(CC(=O)CN1CCCC1C(OC)C(C)C(=O)NC(C)C(O)c1ccccc1)OC)N(C)C(=O)C(NC(=O)C(C(C)C)N(C)C(=O)OCc1ccc(NC(=O)C(CCCNC(N)=O)NC(=O)C(NC(=O)CCc2ccc(C=O)c(C=O)c2)C(C)C)cc1)C(C)C]

11. A compound of the following formula (III):
Ab-(OPA-L-D)p (III)
wherein
Ab is a cell binding reagent, wherein said cell binding reagent includes IgGs, bi-specific antibody, antibody fragment such as Fab, Fab′, F(ab′)2 and scFv, Heavy-chain only antibody or Nanobody;
OPA is 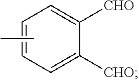
L includes alkyl, alkenyl, alkynyl, cycloalkyl, cycloalkenyl, cycloalkynyl, heterocycloalkyl, heterocycloalkenyl, heterocycloalkynyl, aryl, heteroaryl, alkylene, alkenylene, alkynylene, arylene, heteroarylene, cycloalkylene, heterocycloalkylene; —(CH2)m-, wherein one or more CH2 are replaced by one or more groups selected from NH and C═O, m is an integer of 0-20; —(CH2)n-heteroaryl, wherein one or more CH2 are replaced by one or more groups selected from NH, C═O and —O—, n is an integer of 0-20; —(CH2)o-heterocycloalkyl-(CH2)o-CH3, wherein one or more CH2 are replaced by one or more groups selected from NH and C═O, o is an integer of 0-20; —(CH2)q-cycloalkyl-(CH2)q-CH3, wherein one or more CH2 are replaced by one or more groups selected from NH and C═O, p is an integer of 0-20; —(CH2)r-cycloalkyl-(CH2)r-heteroaryl, wherein one or more CH2 are replaced by one or more groups selected from NH and C═O, r is an integer of 0-20; peptide; oligosaccharide, polyethylene glycol (PEG), and the combinations thereof, and said alkyl, alkenyl, alkynyl, cycloalkyl, cycloalkenyl, cycloalkynyl, heterocycloalkyl, heterocycloalkynyl, aryl, heteroaryl, —CH3, heterocycloalkenyl, alkylene, alkenylene, alkynylene, arylene, heteroarylene, cycloalkylene, heterocycloalkylene are optionally substituted by at least one substituents;
D is independently active reagent, wherein said active reagent includes an anti-cancer reagent such as Mertansine and MMAE; an anti-inflammation reagent; Fluorescein such as FTIC; a peptide; a protein; a nucleotide; an oligonucleotide; a chemotherapy drug; a natural product; an immune modulator; a tubulin-binder; a DNA-alkylating agent; an HSP90 inhibitor; a DNA topoisomerase inhibitor; an anti-epigenetic agent; an HDAC inhibitor; an anti-metabolism agent; a proteasome inhibitor; a peptidomimetic; an siRNA; an antisense DNA; epothilone A, epothilone B, or paclitaxel;
p is a integer refers to the number of active reagent attached to cell binding reagent, wherein p is an integer of 0-15.

12. The compound according to claim 11, wherein L includes
—(CH2)m-, wherein one or more CH2 are replaced by one or more groups selected from NH and C═O, m is an integer of 0-15;
—(CH2)n-heteroaryl, wherein one or more CH2 are replaced by one or more groups selected from NH, C═O and —O—, n is an integer of 0-20, and the heteroaryl group has 5 to 10 ring members;
—(CH2)o-C₃₋₇heterocycloalkyl-(CH2)o-CH3, wherein one or more CH2 are replaced by one or more groups selected from NH and C═O, o is an integer of 0-15;
—(CH2)q-C₃₋₇cycloalkyl-(CH2)q-CH3, wherein one or more CH2 are replaced by one or more groups selected from NH and C═O, q is an integer of 0-15;
—(CH2)r-C₃₋₇cycloalkyl-(CH2)r-heteroaryl, wherein one or more CH2 are replaced by one or more groups selected from NH and C═O, r is an integer of 0-15, and the heteroaryl group has 5 to 10 ring members, and
said cycloalkyl, heterocycloalkyl, —CH3 and heteroaryl are optionally substituted by oxo and halogen.

13. The compound according to claim 11, wherein L includes
—(CH2)m-, wherein one or more CH2 are replaced by one or more groups selected from NH and C═O, m is an integer of 0-10;
—(CH2)n-heteroaryl, wherein one or more CH2 are replaced by one or more groups selected from NH, C═O and —O—, n is an integer of 0-20, and the 5 to 10 membered heteroaryl has 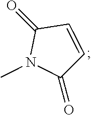
—(CH2)o-piperazinyl-(CH2)o-CH3, wherein one or more CH2 are replaced by one or more groups selected from NH and C═O, o is an integer of 0-10;
—(CH2)q-cyclohexyl-(CH2)q-CH3, wherein one or more CH2 are replaced by one or more groups selected from NH and C═O, q is an integer of 0-10;
—(CH2)r-cyclohexyl-(CH2)r-heteroaryl, wherein one or more CH2 are replaced by one or more groups selected from NH and C═O, r is an integer of 0-10, and the 5 to 10 membered heteroaryl has 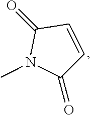
and
said —CH3- is optionally substituted by halogen.

14. The compound according to claim 11, wherein L includes
—(CH2)m-, wherein one or more CH2 are replaced by one or more groups selected from NH and C═O, m is an integer of 0-10;
—(CH2)n-heteroaryl, wherein no more than eight CH2 are replaced by one or more groups selected from NH, C═O and —O—, n is an integer of 0-18, and the 5 to 10 membered heteroaryl has 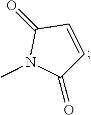
—(CH2)o-piperazinyl-(CH2)o-CH3, wherein one CH2 is replaced by one group selected from NH and C═O, o is an integer of 0-5;
—(CH2)q-cyclohexyl-(CH2)q-CH3, wherein no more than six CH2 are replaced by one or more groups selected from NH and C═O, q is an integer of 0-8;
—(CH2)r-cyclohexyl-(CH2)r-heteroaryl, wherein no more than five CH2 are replaced by one or more groups selected from NH and C═O, r is an integer of 0-6, and the 5 to 10 membered heteroaryl has 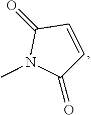
and
said —CH3- is optionally substituted by Cl.

15. The compound according to claim 11, wherein the OPA-L-D is selected from the group consisting of:

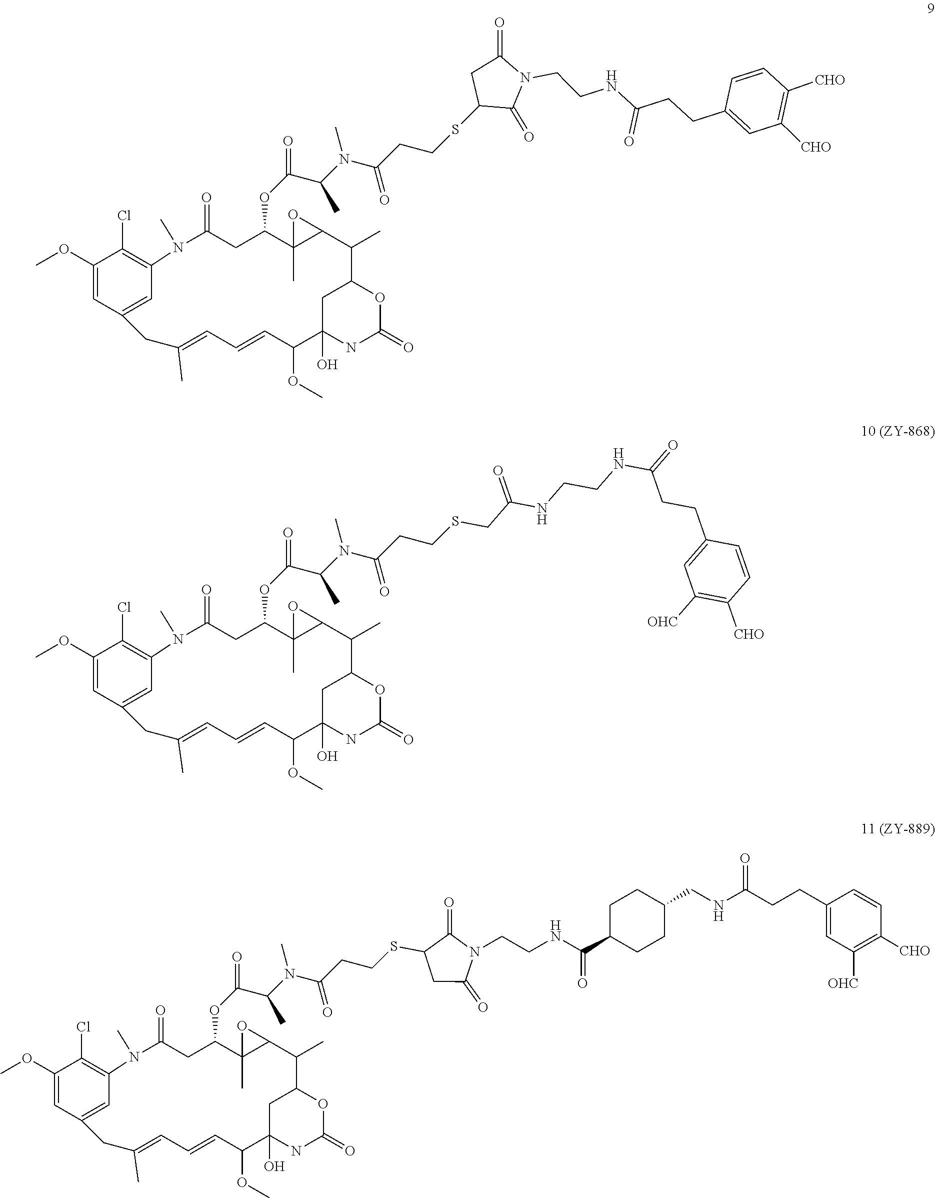 [SMILES: COc1cc2cc(c1Cl)N(C)C(=O)C[C@H](OC(=O)[C@H](C)N(C)C(=O)CCSC1CC(=O)N(CCNC(=O)CCc3ccc(C=O)c(C=O)c3)C1=O)C1(C)OC1C(C)C1CC(O)(NC(=O)O1)C(OC)/C=C/C=C(\C)C2 ; COc1cc2cc(c1Cl)N(C)C(=O)C[C@H](OC(=O)[C@H](C)N(C)C(=O)CCSCC(=O)NCCNC(=O)CCc1ccc(C=O)c(C=O)c1)C1(C)OC1C(C)C1CC(O)(NC(=O)O1)C(OC)/C=C/C=C(\C)C2 ; COc1cc2cc(c1Cl)N(C)C(=O)C[C@H](OC(=O)[C@H](C)N(C)C(=O)CCSC1CC(=O)N(CCNC(=O)[C@H]3CC[C@H](CNC(=O)CCc4ccc(C=O)c(C=O)c4)CC3)C1=O)C1(C)OC1C(C)C1CC(O)(NC(=O)O1)C(OC)/C=C/C=C(\C)C2]

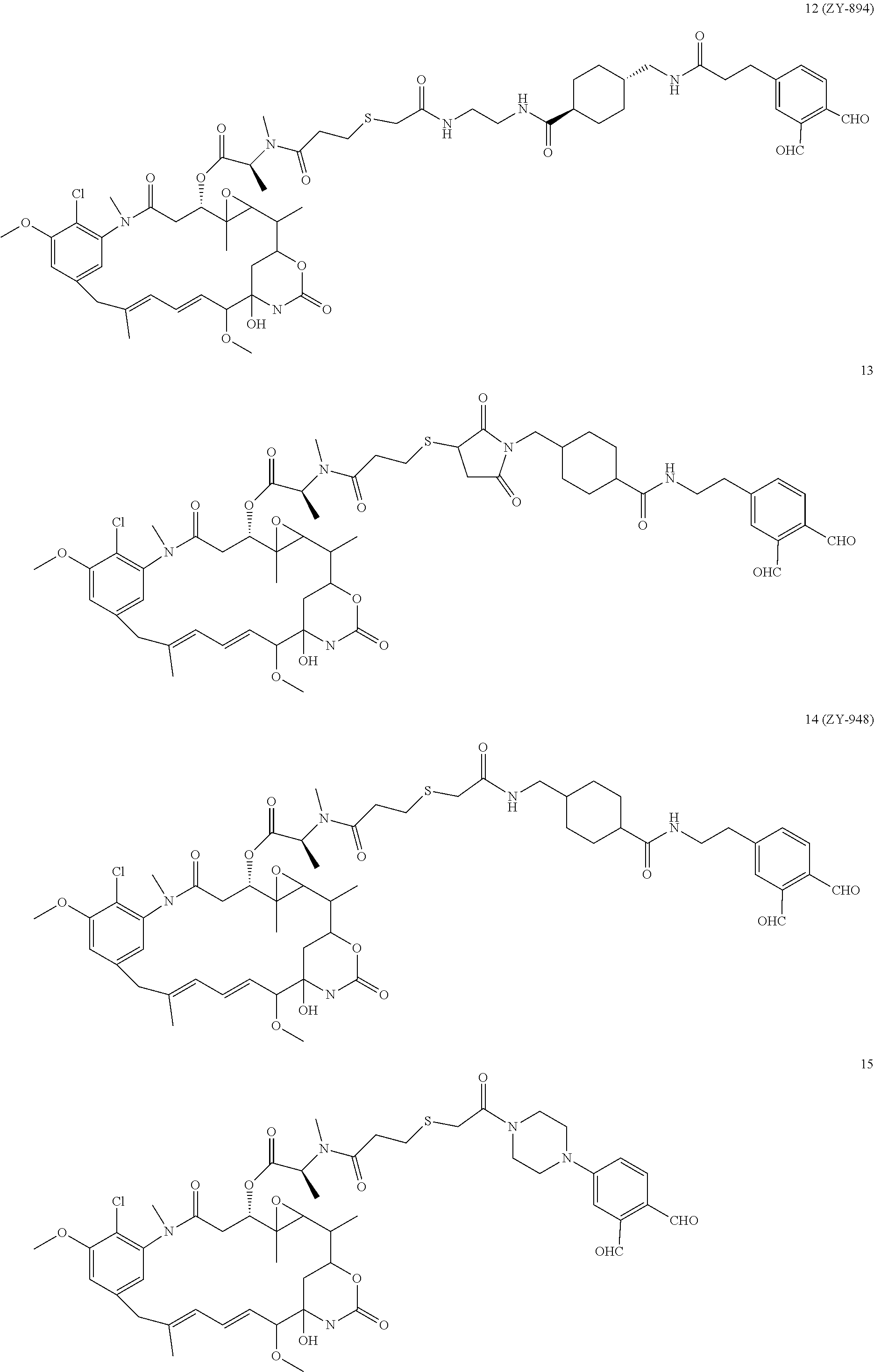

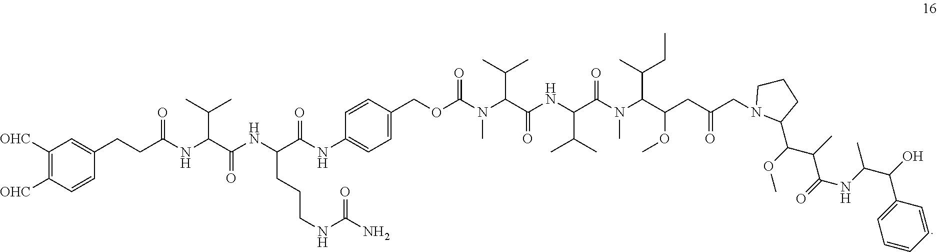 [SMILES: CCC(C)C(C(CC(=O)CN1CCCC1C(OC)C(C)C(=O)NC(C)C(O)c1ccccc1)OC)N(C)C(=O)C(NC(=O)C(C(C)C)N(C)C(=O)OCc1ccc(NC(=O)C(CCCNC(N)=O)NC(=O)C(NC(=O)CCc2ccc(C=O)c(C=O)c2)C(C)C)cc1)C(C)C]

16. (canceled)

17. A process for the preparation of conjugate of the following formula (III):
Ab-(OPA-L-D)p (III)
Wherein the conjugate comprises D linked to Ab through the reaction of primary amine on Ab and OPA-L, the process comprising the steps of:
(a) contacting D with OPA-L to covalently attach the OPA-L to D and therefore prepare OPA-L-D, wherein D, OPA and L are defined in claim 11;
(b) conjugating Ab to OPA-L-D by reacting the OPA-L-D with Ab to prepare the conjugate of formula (III), wherein Ab and P are defined in claim 11; and
(c) purifying the conjugate of formula (III) with down-stream steps such as buffer exchange or column purification.

18. The process according to claim 17, wherein step (a) is carried out in a buffer with pH 7-12.

19. The process according to claim 17, wherein step (b) is carried out in a buffer with pH 4-7, under 4-37° C. for 1 h-24 h.

20. The process according to claim 17, wherein the buffer in step (b) contains 2.5%-20% organic co-solvent, and conjugation in step (b) is carried out with 5 eq to 30 eq of OPA-L-D to Ab.
